(19) United States
(12) Patent Application Publication
(10) Pub. No.: US 2022/0144769 A1
(43) Pub. Date: May 12, 2022

(54) COMPOUND

(71) Applicant: Sumitomo Chemical Company, Limited, Tokyo (JP)

(72) Inventors: Yuji ASATSU, Osaka-shi, Osaka (JP); Shoichi OZAWA, Tama-City, Tokyo (JP); Ai KOBASHI, Niihama-shi, Ehime (JP)

(21) Appl. No.: 17/434,004

(22) PCT Filed: Feb. 21, 2020
(86) PCT No.: PCT/JP2020/007179

§ 371 Date: Aug. 25, 2021

(30) Foreign Application Priority Data
Feb. 28, 2019 (JP) 2019-035795
Jul. 8, 2019 (JP) 2019-126918

(51) Int. Cl. C07D 207/06; C07D 295/155; C07D 207/20; C07D 319/06; C07D 209/18; C07C 255/46; C07D 205/04; C07F 7/18; C07C 255/30

(52) U.S. Cl. CPC C07D 207/06; C07D 295/155; C07D 207/20; C07D 319/06; C07C 255/30; C07C 255/46; C07D 205/04; C07F 7/1804; C07D 209/18

(57) ABSTRACT

A compound represented by Formula (I) is provided:

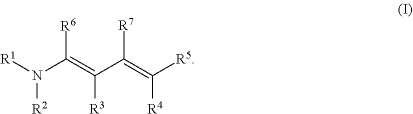

In Formula (I), R³, R⁴, and R⁵ each independently represent an electron-withdrawing group; R¹, R², R⁶, and R⁷ each independently represent a hydrogen atom, a heterocyclic group, a halogen atom, a nitro group, a cyano group, a hydroxy group, a thiol group, a carboxy group, —SCF₃, —SF₅, —SF₃, —SO₃H, —SO₂H, an aliphatic hydrocarbon group having 1 to 25 carbon atoms which may have a substituent, or an aromatic hydrocarbon group having 6 to 18 carbon atoms which may have a substituent; and R¹ and R², R¹ and R⁶, R⁶ and R⁷, and R⁴ and R⁵ may be linked to each other to form a ring.

### TECHNICAL FIELD

The present invention relates to a compound.

### BACKGROUND ART

In the related art, an ultraviolet absorber has been used in various applications and products in order to protect human bodies and resin materials from deterioration due to ultraviolet rays. The ultraviolet absorber is roughly divided into an inorganic ultraviolet absorber and an organic absorber. While the inorganic ultraviolet absorber has excellent durability such as light resistance and heat resistance, it tends to be inferior in absorption wavelength control and compatibility with an organic material. On the other hand, although the organic ultraviolet absorber is inferior in durability to the inorganic ultraviolet absorber, the organic ultraviolet absorber can control an absorption wavelength, compatibility with an organic material, and the like from the degree of freedom of a molecular structure in the organic ultraviolet absorber, and is used in a wide range of fields such as sunscreens, paints, optical materials, building materials, and automobile materials.

Examples of the organic ultraviolet absorber generally include compounds having a triazole skeleton, a benzophenone skeleton, a triazine skeleton, and a cyanoacrylate skeleton. However, since many of the organic ultraviolet absorbers having a skeleton have a maximum absorption wavelength (λ max) of 360 nm or less, the organic ultraviolet absorbers cannot efficiently absorb an ultraviolet to near-ultraviolet region having a wavelength of 380 to 400 nm, and it is necessary to increase the amount used to sufficiently absorb light in this region. In addition, in a case where many compounds having a skeleton have a broad absorption spectrum and sufficiently absorb light having a wavelength of 380 to 400 nm, there is a problem that absorption occurs not only in a wavelength region of 380 to 400 nm but also in light having a wavelength of 420 nm or more, and thereby a composition containing an ultraviolet absorber is colored.

As means for solving the above problems, for example, Patent Document 1 proposes a compound having a merocyanine skeleton as represented by the following formula as an organic ultraviolet absorber. Patent Document 1 discloses that a film containing a compound having a merocyanine skeleton represented by the following formula has low light transmittance in the vicinity of a wavelength of 390 nm.

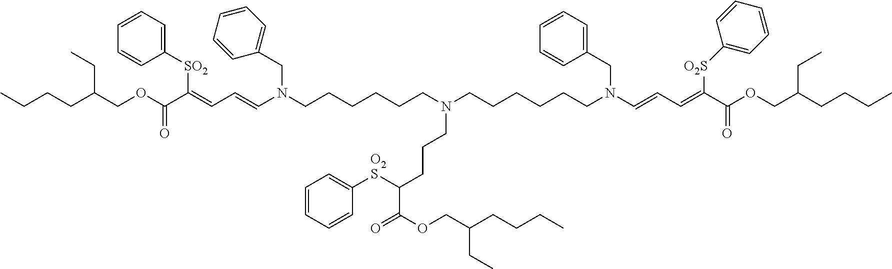 [SMILES: CCCCC(CC)COC(=O)/C(=C/C=C/N(CCCCCCN(CCCCCCN(/C=C/C=C(/C(=O)OCC(CC)CCCC)S(=O)(=O)c1ccccc1)Cc1ccccc1)CCCC(C(=O)OCC(CC)CCCC)S(=O)(=O)c1ccccc1)Cc1ccccc1)S(=O)(=O)c1ccccc1]

### PRIOR ART DOCUMENT

### Patent Document

Patent Document 1: JP-A-2010-111823

### SUMMARY OF THE INVENTION

### Problems to be Solved by the Invention

However, a compound having a merocyanine skeleton has low durability (particularly weather resistance), and thus is difficult to apply to applications requiring severe weather resistance.

An object of the present invention is to provide a novel compound having a merocyanine skeleton which efficiently absorbs light having a wavelength of 380 to 400 nm and can be used as an ultraviolet to near-ultraviolet absorber having good weather resistance.

### Means for Solving the Problems

The present invention includes the following inventions.

[1] A compound represented by Formula (I):

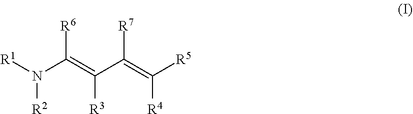

[in Formula (I),

R³, R⁴, and R⁵ each independently represent an electron-withdrawing group;

R¹, R², R⁶ and R⁷ each independently represent a hydrogen atom, a heterocyclic group, a halogen atom, a nitro group, a cyano group, a hydroxy group, a thiol group, a carboxy group, —SCF₃, —SF₅, —SF₃, —SO₃H, —SO₂H, an aliphatic hydrocarbon group having 1 to 25 carbon atoms which may have a substituent, or an aromatic hydrocarbon group having 6 to 18 carbon atoms which may have a substituent, and —CH₂— or —CH═ contained in the aliphatic hydrocarbon group or the aromatic hydrocarbon group may be substituted with —NR^{1A}—, —SO₂—, —CO—, —O—, —COO—, —OCO—, —CONR^{2A}—, —NR^{3A}—CO—, —S—, —SO—, —CF₂— or —CHF—;

R^{1A}, R^{2A}, and R^{3A} each independently represent a hydrogen atom or an alkyl group having 1 to 6 carbon atoms;

R¹ and R² may be linked to each other to form a ring;

R¹ and R⁶ may be linked to each other to form a ring;

R⁶ and R⁷ may be linked to each other to form a ring; and

R⁴ and R⁵ may be linked to each other to form a ring].

[2] The compound according to [1], wherein R³ is a nitro group, a cyano group, —F, —OCF₃, —SCF₃, —SF₅, —SF₃, —SO₂—R²²² (R²²² represents a hydrogen atom, an alkyl group having 1 to 25 carbon atoms which may have a substituent, or an aromatic hydrocarbon group having 6 to 18 carbon atoms which may have a substituent), a fluoroalkyl group, or a fluoroaryl group.

[3] The compound according to [1] or [2], wherein R³ is a cyano group.

[4] The compound according to any one of [1] to [3], wherein at least one selected from R⁴ and R⁵ is a cyano group, a nitro group, —OCF₃, —SCF₃, —SF₅, —CO—O—R²²², —SO₂—R²²² (R²²² represents a hydrogen atom, an alkyl group having 1 to 25 carbon atoms which may have a substituent, or an aromatic hydrocarbon group having 6 to 18 carbon atoms which may have a substituent), a fluoroalkyl group, or a fluoroaryl group.

[5] The compound according to any one of [1] to [4], wherein at least one selected from R⁴ and R⁵ is a cyano group.

[6] The compound according to any one of [1] to [5], wherein both R⁴ and R⁵ are a cyano group.

[7] The compound according to any one of [1] to [6], wherein R¹ and R² are each independently an aliphatic hydrocarbon group which may have a substituent.

[8] The compound according to any of [1] to [6], wherein R¹ and R² are linked to each other to form a ring.

[9] The compound according to [8], wherein the ring formed by linking R¹ and R² to each other is a ring having no unsaturated bond.

[10] The compound according to [1] to [9], wherein the compound represented by Formula (I) is a compound represented by Formula (II):

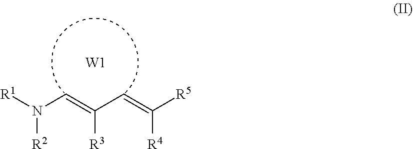

[in the formula, R¹, R², R³, R⁴, and R⁵ have the same meaning as described above; and

a ring W¹ represents a ring structure having at least one double bond as a constituent element of the ring and having no aromaticity].

[11] The compound according to [10], wherein the ring W¹ has a 5 to 7-membered ring structure.

[12] The compound according to [10] or [11], wherein the ring W¹ is a 6-membered ring structure.

[13] The compound according to any of [1] to [12], wherein λ max≥370 nm,

(λ max represents a maximum absorption wavelength [nm] of the compound represented by Formula (I)).

[14] The compound according to any one of [1] to [13], which satisfies Formula (B),

ε(λ max)/ε(λ max+30 nm)≥5 (B)

(in the formula, s (λ max) represents a gram absorption coefficient at a maximum absorption wavelength [nm] of the compound represented by Formula (I), and ε (λ max+30 nm) represents a gram absorption coefficient at a wavelength [nm] of (maximum absorption wavelength [nm]+30 nm) of the compound represented by Formula (I)).

[15] A composition containing the compound according to any one of [1] to [14].

### Effect of the Invention

The present invention provides a novel compound having a merocyanine skeleton having high absorption selectivity to short-wavelength visible light having a wavelength of 380 to 400 nm. In addition, the compound of the present invention has good weather resistance.

### MODE FOR CARRYING OUT THE INVENTION

The compound of the present invention is a compound having a structure represented by Formula (I) (hereinafter, may be referred to as a compound (I)).

<Compound (I)>

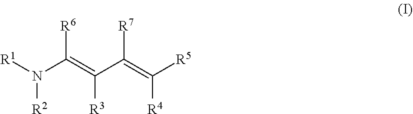

[in Formula (I),

R³, R⁴, and R⁵ each independently represent an electron-withdrawing group;

R¹, R², R⁵ and R⁷ each independently represent a hydrogen atom, a heterocyclic group, a halogen atom, a nitro group, a cyano group, a hydroxy group, a thiol group, a carboxy group, —SCF₃, —SF₅, —SF₃, —SO₃H, —SO₂H, an aliphatic hydrocarbon group having 1 to 25 carbon atoms which may have a substituent, or an aromatic hydrocarbon group having 6 to 18 carbon atoms which may have a substituent, and —CH₂— or —CH═ contained in the aliphatic hydrocarbon group or the aromatic hydrocarbon group may be substituted with —NR^{1A}—, —SO₂—, —CO—, —O—, —COO—, —OCO—, —CONR^{2A}—, —S—, —SO—, —SO₂—, —CF₂— or —CHF—;

R^{1A}, R^{2A}, and R^{3A} each independently represent a hydrogen atom or an alkyl group having 1 to 6 carbon atoms;

R¹ and R² may be linked to each other to form a ring;

R¹ and R⁶ may be linked to each other to form a ring;

R⁶ and R⁷ may be linked to each other to form a ring; and

R⁴ and R⁵ may be linked to each other to form a ring].

In the present specification, the number of carbon atoms does not include the number of carbon atoms of the substituent, and refers to the number of carbon atoms before substitution when —CH₂— or —CH═ is substituted, for example, as described above.

Examples of the electron-withdrawing group represented by R³, R⁴, and R⁵ include a halogen atom, a nitro group, a cyano group, a carboxy group, a halogenated alkyl group, a halogenated aryl group, —OCF₃, —SCF₃, —SF₅, —SF₃, —SO₃H, and —SO₂H, and a group represented by Formula (X-1).

*—X¹—R²²² (X-1)

[in Formula (X-1),

X¹ represents —CO—, —COO—, —OCO—, —CS—, —CSS—, —COS—, —CSO—, —SO₂—, —NR²²³CO—, or —CONR²²⁴—;

R²²² represents a hydrogen atom, an alkyl group having 1 to 25 carbon atoms which may have a substituent, or an aromatic hydrocarbon group having 6 to 18 carbon atoms which may have a substituent;

R²²³ and R²²⁴ each independently represent a hydrogen atom, an alkyl group having 1 to 6 carbon atoms, or a phenyl group; and

* represents a bond].

Examples of the halogen atom include a fluorine atom, a chlorine atom, a bromine atom, and an iodine atom.

Examples of the halogenated alkyl group include a fluoroalkyl group such as a trifluoromethyl group, a perfluoroethyl group, a perfluoropropyl group, a perfluoroisopropyl group, a perfluorobutyl group, a perfluorosec-butyl group, a perfluorotert-butyl group, a perfluoropentyl group, and a perfluorohexyl group, and a perfluoroalkyl group is preferable. The number of carbon atoms of the halogenated alkyl group is usually 1 to 25, and preferably 1 to 12. The halogenated alkyl group may be linear or branched.

Examples of the halogenated aryl group include a fluorophenyl group, a chlorophenyl group, and a bromophenyl group, and the halogenated aryl group is preferably a fluoroaryl group, and more preferably a perfluoroaryl group. The number of carbon atoms of the halogenated aryl group is usually 6 to 18, and preferably 6 to 12.

X¹ is preferably —COO— or —SO₂—.

Examples of the alkyl group having 1 to 25 carbon atoms represented by R²²² include linear or branched alkyl groups having 1 to 25 carbon atoms such as a methyl group, an ethyl group, an n-propyl group, an isopropyl group, an n-butyl group, a tert-butyl group, a sec-butyl group, an n-pentyl group, an n-hexyl group, a 1-methylbutyl group, a 3-methylbutyl group, a n-octyl group, a n-decyl group, and a 2-hexyl-octyl group. R²²² is preferably an alkyl group having 1 to 12 carbon atoms.

Examples of the substituent that the alkyl group having 1 to 25 carbon atoms represented by 8²²² may have include a halogen atom and a hydroxy group.

Examples of the aromatic hydrocarbon group having 6 to 18 carbon atoms represented by R²²² include an aryl group having 6 to 18 carbon atoms such as a phenyl group, a naphthyl group, an anthracenyl group, and a biphenyl group; and an aralkyl group having 7 to 18 carbon atoms such as a benzyl group, a phenylethyl group, and a naphthylmethyl group.

Examples of the substituent that the aromatic hydrocarbon group having 6 to 18 carbon atoms represented by R²²² may have include a halogen atom and a hydroxy group.

Examples of the alkyl group having 1 to 6 carbon atoms represented by R²²³ and R²²⁴ include linear or branched alkyl groups having 1 to 6 carbon atoms such as a methyl group, an ethyl group, an n-propyl group, an isopropyl group, an n-butyl group, a tert-butyl group, a sec-butyl group, an n-pentyl group, an n-hexyl group, a 1-methylbutyl group, and a 3-methylbutyl group.

R³ is preferably a nitro group, a cyano group, —F, —OCF₃, —SCF₃, —SF₅, —SF₃, —SO₂—R²²² (R²²² represents a hydrogen atom, an alkyl group having 1 to 25 carbon atoms which may have a substituent, or an aromatic hydrocarbon group having 6 to 18 carbon atoms which may have a substituent), a fluoroalkyl group (preferably, a fluoroalkyl group having 1 to 25 carbon atoms), or a fluoroaryl group (preferably, a fluoroaryl group having 6 to 18 carbon atoms), more preferably a cyano group, —F, —OCF₃, —SCF₃, or a fluoroalkyl group (preferably, a fluoroalkyl group having 1 to 25 carbon atoms), and still more preferably a cyano group.

At least one selected from R⁴ and R⁵ is preferably a cyano group, a nitro group, —OCF₃, —SCF₃, —SF₅, —CO—O—R²²², —SO₂—R²²² (R²²² represents a hydrogen atom, an alkyl group having 1 to 25 carbon atoms which may have a substituent, or an aromatic hydrocarbon group having 6 to 18 carbon atoms which may have a substituent), a fluoroalkyl group (preferably, a fluoroalkyl group having 1 to 25 carbon atoms), or a fluoroaryl group (preferably, a fluoroaryl group having 6 to 18 carbon atoms). A cyano group, a nitro group, —CO—O—R²²² or —SO₂—R²²² is more preferable, and a cyano group is still more preferable.

R⁴ and R⁵ preferably have the same structure.

R⁴ and R⁵ may be bonded to each other to form a ring. The ring formed by bonding R⁴ and R⁵ to each other may be a single ring or a fused ring, but is preferably a single ring. The ring formed by bonding R⁴ and R⁵ to each other may contain a hetero atom (nitrogen atom, oxygen atom, or sulfur atom) or the like as a constituent element of the ring.

The ring formed by bonding R⁴ and R⁵ to each other is usually a 3 to 10-membered ring, preferably a 5 to 7-membered ring, and more preferably a 5-membered ring or a 6-membered ring.

Examples of the ring formed by bonding R⁴ and R⁵ to each other include the structures described below.

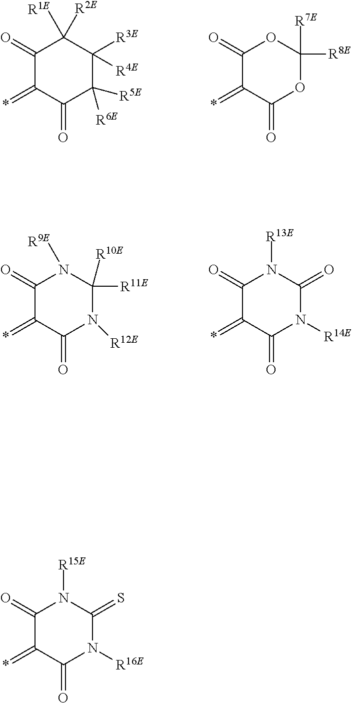

[in the formula, * represents a bond to a carbon atom; and R^{1E} to R^{16E} each independently represent a hydrogen atom or a substituent].

The ring formed by bonding R⁴ and R⁵ to each other may have a substituent (R^{1E} to R^{16E} in the above formula).

Examples of the substituents represented by R^{1E} to R^{16E} include a halogen atom such as a fluorine atom, a chlorine atom, a bromine atom, or an iodine atom; an alkyl group having 1 to 12 carbon atoms, such as a methyl group, an ethyl group, a propyl group, a butyl group, a pentyl group, a hexyl group, a heptyl group, an octyl group, or a nonyl group; a halogenated alkyl group having 1 to 12 carbon atoms, such as a fluoromethyl group, a difluoromethyl group, a trifluoromethyl group, a 2-fluoroethyl group, a 2,2-difluoroethyl group, a 2,2,2-trifluoroethyl group, a 1,1,2,2-tetrafluoroethyl group, and a 1,1,2,2,2-pentafluoroethyl group; an alkoxy group having 1 to 12 carbon atoms, such as a methoxy group, an ethoxy group, a propoxy group, a butoxy group, a pentyloxy group, or a hexyloxy group; an alkylthio group having 1 to 12 carbon atoms, such as a methylthio group, an ethylthio group, a propylthio group, a butylthio group, a pentylthio group, and a hexylthio group; a fluorinated alkoxy group having 1 to 12 carbon atoms, such as a monofluoromethoxy group, a difluoromethoxy group, a trifluoromethoxy group, a 2-fluoroethoxy group, and a 1,1,2,2,2-pentafluoroethoxy group; an amino group which may be substituted with an alkyl group having 1 to 6 carbon atoms such as an amino group, a methylamino group, an ethylamino group, a dimethylamino group, a diethylamino group, or a methylethyl group; an alkylcarbonyloxy group having 2 to 12 carbon atoms, such as a methylcarbonyloxy group and an ethylcarbonyloxy group; an alkylsulfonyl group having 1 to 12 carbon atoms, such as a methylsulfonyl group and an ethylsulfonyl group; an arylsulfonyl group having 6 to 12 carbon atoms, such as a phenylsulfonyl group; a cyano group; a nitro group; a hydroxyl group; a thiol group; a carboxy group; —SF₃; and —SF₅.

The R^{1E} to R^{16E} are each independently preferably an alkyl group having 1 to 12 carbon atoms, more preferably an alkyl group having 1 to 6 carbon atoms, and still more preferably a methyl group.

Examples of the aliphatic hydrocarbon group having 1 to 25 carbon atoms represented by R¹, R², R⁶, and R⁷ include a linear or branched alkyl group having 1 to 25 carbon atoms such as a methyl group, an ethyl group, an n-propyl group, an isopropyl group, an n-butyl group, a tert-butyl group, a sec-butyl group, an n-pentyl group, an isopentyl group, an n-hexyl group, an isohexyl group, an n-octyl group, an isooctyl group, an n-nonyl group, an isononyl group, an n-decyl group, an isodecyl group, an n-dodecyl group, an isododecyl group, an undecyl group, a lauryl group, a myristyl group, a cetyl group, and a stearyl group; a cycloalkyl group having 3 to 25 carbon atoms such as a cyclopropyl group, a cyclobutyl group, a cyclopentyl group and a cyclohexyl group; and a cycloalkylaikyl group having 4 to 25 carbon atoms such as a cyclohexylmethyl group.

The aliphatic hydrocarbon groups having 1 to 25 carbon atoms represented by R¹, R², R⁶, and R⁷ are each independently preferably an alkyl group having 1 to 15 carbon atoms, and more preferably an alkyl group having 1 to 12 carbon atoms.

Examples of the substituent that the aliphatic hydrocarbon groups having 1 to 25 carbon atoms represented by R¹, R², R⁶, and R⁷ may have include a halogen atom, a hydroxyl group, a nitro group, a cyano group, —SO₃H, a thiol group, and an amino group.

—CH₂— or —CH═ contained in the aliphatic hydrocarbon group having 1 to 25 carbon atoms represented by R¹, R², R⁵, and R⁷ may be substituted with —SO₂—, —CO—, —O—, —COO—, —OCO—, —CONR^{2A}—, —NR^{3A}—CO—, —S—, —SO—, —CF₂— or —CHF—.

When —CH₂— or —CH═ contained in the aliphatic hydrocarbon group having 1 to 25 carbon atoms is substituted, —O—, —S—, —COO—, or —SO₂— is preferably substituted.

When —CH₂— or —CH═ contained in the aliphatic hydrocarbon group having 1 to 25 carbon atoms is substituted with —O—, the aliphatic hydrocarbon group is preferably an alkoxy group represented by —O—R¹¹¹ (R¹¹¹ is an alkyl group having 1 to 24 carbon atoms which may have a halogen atom). In addition, it may be a polyalkyleneoxy group such as a polyethyleneoxy group or a polypropyleneoxy group.

When —CH₂— or —CH═ contained in the aliphatic hydrocarbon group having 1 to 25 carbon atoms is substituted with —S—, the aliphatic hydrocarbon group is preferably an alkylthio group represented by —S—R¹¹¹ (R¹¹¹ is an alkyl group having 1 to 24 carbon atoms which may have a halogen atom).

When —CH₂— or —CH═ contained in the aliphatic hydrocarbon group having 1 to 25 carbon atoms is substituted with —COO—, the aliphatic hydrocarbon group is preferably a group represented by —COO—R¹¹¹ (R¹¹¹ is an alkyl group having 1 to 24 carbon atoms which may have a halogen atom).

When —CH₂— or —CH═ contained in the aliphatic hydrocarbon group having 1 to 25 carbon atoms is substituted with —SO₂—, the aliphatic hydrocarbon group is preferably a group represented by —SO₂—R¹¹¹ (R¹¹¹ is an alkyl group having 1 to 24 carbon atoms which may have a halogen atom), and may be —SO₂CHF₂, —SO₂CH₂F, or the like.

Examples of the aromatic hydrocarbon group having 6 to 18 carbon atoms represented by R¹, R², and R⁷ include an aryl group having 6 to 18 carbon atoms such as a phenyl group, a naphthyl group, an anthracenyl group, a tetracenyl group, a pentacenyl group, a phenanthryl group, a chrysenyl group, a triphenylenyl group, a tetraphenyl group, a pyrenyl group, a perylenyl group, a coronenyl group, and a biphenyl group; and an aralkyl group having 7 to 18 carbon atoms such as a benzyl group, a phenylethyl group, and a naphthylmethyl group, and an aryl group having 6 to 18 carbon atoms is preferable, and a phenyl group or a benzyl group is more preferable.

Examples of the substituent that the aromatic hydrocarbon group having 6 to 18 carbon atoms represented by R¹, R², R⁶, and R⁷ may have include a halogen atom; a hydroxyl group; a thiol group; an amino group; a nitro group; a cyano group; and a —SO₃H group.

—CH₂— or —CH═ contained in the aromatic hydrocarbon group having 6 to 18 carbon atoms represented by R′, R², R⁶, and R⁷ may be substituted with —SO₂—, —CO—, —O—, —COO—, —OCO—, —CONR^{2A}—, —S—, —SO—, —CF₂— or —CHF—.

When —CH₂— or —CH═ contained in the aromatic hydrocarbon group having 6 to 18 carbon atoms is substituted, it is preferably substituted with —O— or —SO₂—.

When —CH₂— or —CH═ contained in the aromatic hydrocarbon group having 6 to 18 carbon atoms is substituted with —O—, the aromatic hydrocarbon group is an aryloxy group having 6 to 17 carbon atoms such as a phenoxy group; a phenoxyethyl group, a phenoxydiethylene glycol group, an arylalkoxy group of a phenoxypolyalkylene glycol group, and the like are preferable.

When —CH₂— or —CH═ contained in the aromatic hydrocarbon group having 6 to 17 carbon atoms is substituted with —SO₂—, the aromatic hydrocarbon group is preferably a group represented by —SO₂—R¹¹² (R¹¹² represents an aryl group having 6 to 17 carbon atoms or an aralkyl group having 7 to 17 carbon atoms).

Examples of the alkyl group having 1 to 6 carbon atoms represented by R^{1A}, R^{2A}, and R^{3A} include the same alkyl group as the alkyl group having 1 to 6 carbon atoms represented by 8223.

Examples of the halogen atom represented by R¹, R², R⁶, and R⁷ include a fluorine atom, a chlorine atom, a bromine atom, and an iodine atom.

Examples of the heterocyclic group represented by R¹, R², R⁶, and R⁷ include aliphatic heterocyclic groups having 3 to 16 carbon atoms and aromatic heterocyclic groups having 3 to 16 carbon atoms, such as a pyridyl group, a pyrrolidyl group, a tetrahydrofurfuryl group, a tetrahydrothiophene group, a pyrrole group, a furyl group, a thiopheno group, a piperidine group, a tetrahydropyranyl group, a tetrahydrothiopyranyl group, a thiopyranyl group, an imidazolino group, a pyrazole group, an oxazole group, a thiazolyl group, a dioxanyl group, a morpholino group, a thiazinyl group, a triazole group, a tetrazole group, a dioxolanyl group, a pyridazinyl group, a pyrimidinyl group, a pyrazinyl group, an indolyl group, an isoindolyl group, benzoimidazolyl group, a purinyl group, a benzotriazolyl group, a quinolinyl group, an isoquinoxalinyl group, a quinazolinyl group, a quinoxalinyl group, a cinnolinyl group, a pteridinyl group, a benzopyranyl group, an anthryl group, an acridinyl group, a xanthenyl group, a carbazolyl group, a tetrasenyl group, a porfinyl group, a chlorinyl group, corinyl group, an adenyl group, a guanyl group, a cytosyl group, a timyl group, an uracil group, a quinolyl group, a thiophenyl group, an imidazolyl group, an oxazolyl group, and a thiazolyl group. It is preferable that the heterocyclic groups represented by R¹, R², R⁶, and R⁷ are each independently a pyrrolidyl group, a piperidyl group, a tetrahydrofurfuryl group, a tetrahydropyranyl group, a tetrahydrothiopheno group, a tetrahydrothiopyranyl group, or a pyridyl group.

The ring formed by bonding R¹ and R² to each other contains one nitrogen atom as a constituent element of the ring. The ring formed by bonding R¹ and R² to each other may be a single ring or a fused ring, but is preferably a single ring. The ring formed by bonding R¹ and R² to each other may contain a hetero atom (nitrogen atom, oxygen atom, or sulfur atom) or the like as a constituent element of the ring. The ring formed by bonding R¹ and R² to each other is preferably a ring having no unsaturated bond.

The ring formed by bonding R¹ and R² to each other is usually a 3 to 10-membered ring, preferably a 5 to 7 membered ring, and more preferably a 5-membered ring or a 6-membered ring.

The ring formed by bonding R¹ and R² to each other may have a substituent. Examples of the substituent include the same substituents as those represented by R^{1E}.

Examples of the ring formed by bonding R¹ and R² to each other include the rings described below.

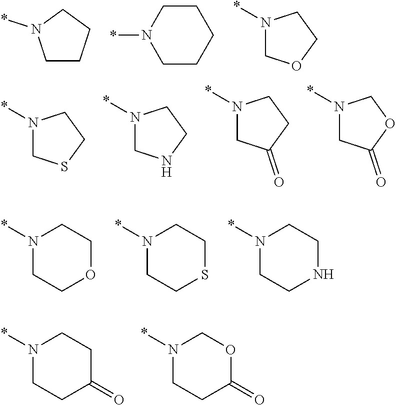

[in the formula, * represents a bond].

R¹ and R⁶ may be linked to each other to form a ring. The ring formed by linking R¹ and R⁶ to each other contains one nitrogen atom as a constituent element of the ring.

The ring formed by bonding R¹ and R⁶ to each other may be a single ring or a fused ring, but is preferably a single ring. The ring formed by bonding R¹ and R⁶ to each other may contain a hetero atom (nitrogen atom, oxygen atom, or sulfur atom) or the like as a constituent element of the ring.

The ring formed by bonding R¹ and R⁶ to each other is usually a 3 to 10-membered ring, preferably a 5 to 7 membered ring, and more preferably a 5-membered ring or a 6-membered ring.

Examples of the ring formed by linking R¹ and R⁶ to each other include the rings described below.

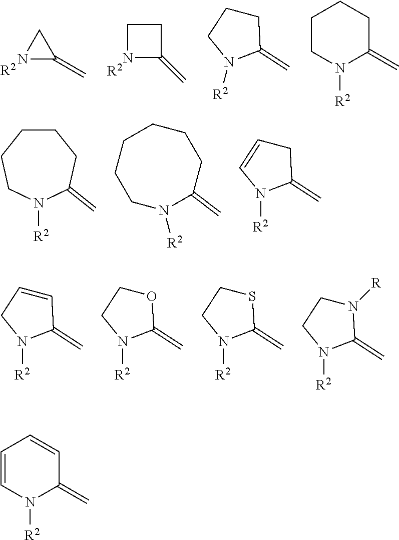

R⁶ and R⁷ may be linked to each other to form a ring. The ring formed by linking R⁶ and R⁷ to each other has at least one double bond as a constituent element of the ring. The double bond contained in the ring formed by linking R⁶ and R⁷ to each other is usually 1 to 4, preferably 1 to 3, more preferably 1 or 2, and still more preferably 1.

The ring formed by linking R⁶ and R⁷ to each other preferably has no aromaticity. The ring formed by linking R⁶ and R⁷ to each other may be a heterocyclic ring containing a hetero atom (for example, an oxygen atom, a sulfur atom, and a nitrogen atom) as a constituent element of the ring.

The ring formed by linking and R⁷ to each other is a ring having 5 to 18 carbon atoms, and is preferably a 5 to 7-membered ring structure, and more preferably a 6-membered ring structure.

The ring formed by linking R⁶ and R⁷ to each other may be a single ring or a fused ring, but is preferably a single ring.

The ring formed by linking R⁶ and R⁷ to each other may have a substituent, and examples of the substituent include the same substituent as the substituent represented by R^{1E}.

Examples of the ring formed by linking R⁶ and R⁷ each other include the rings described below.

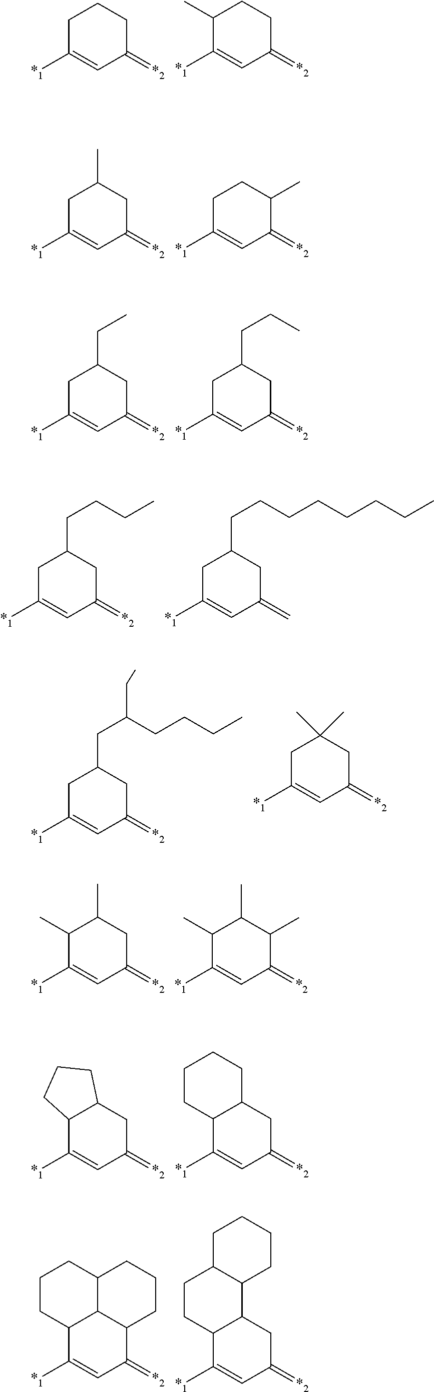

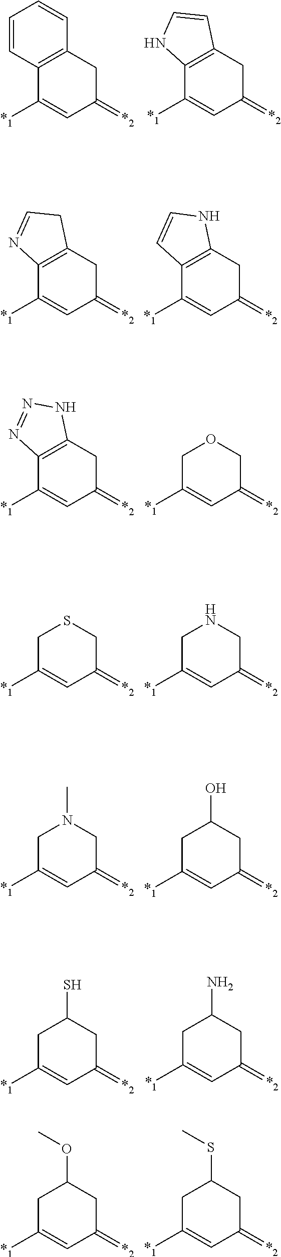

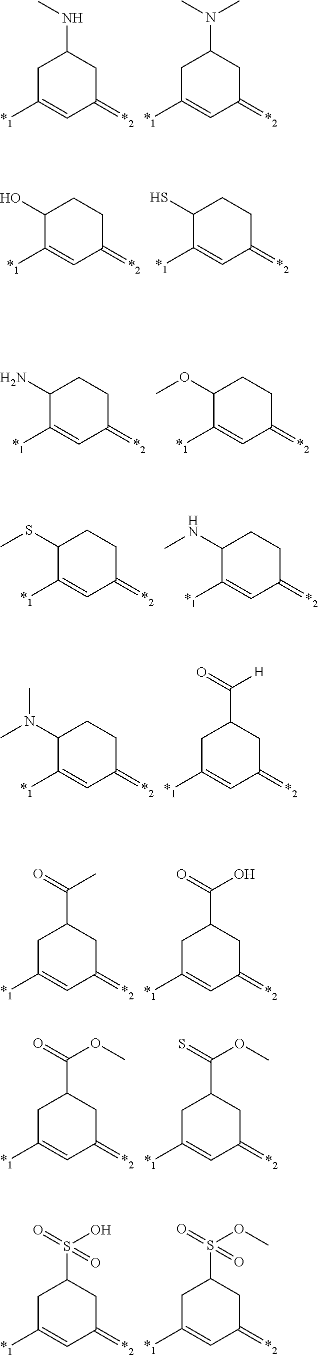

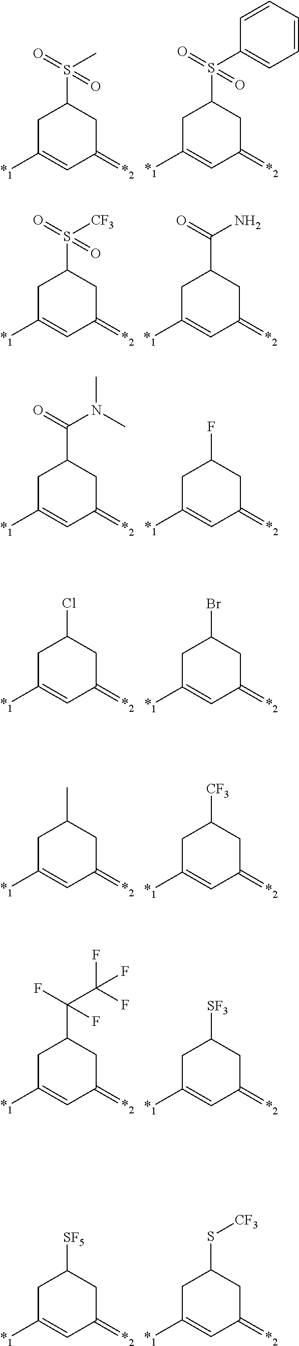

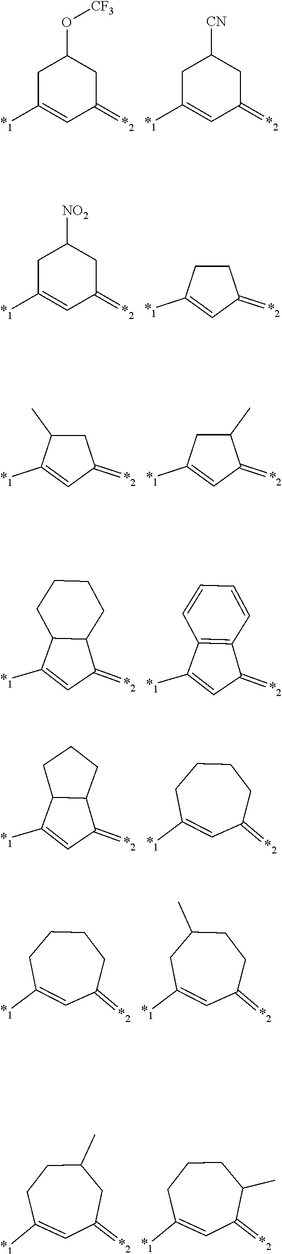

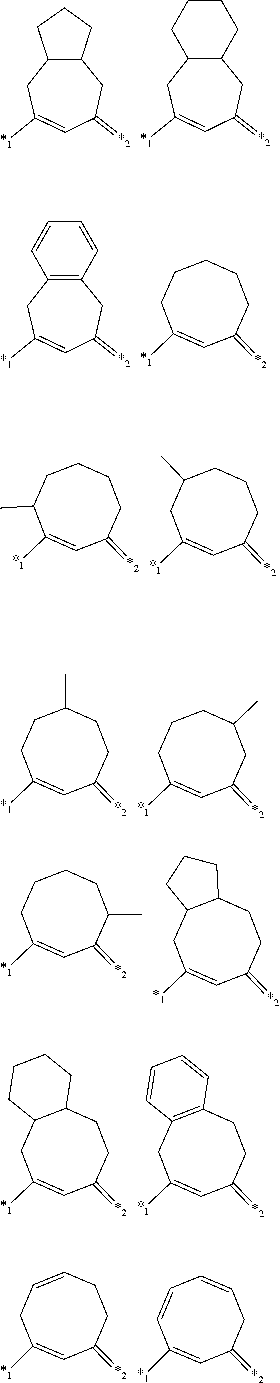

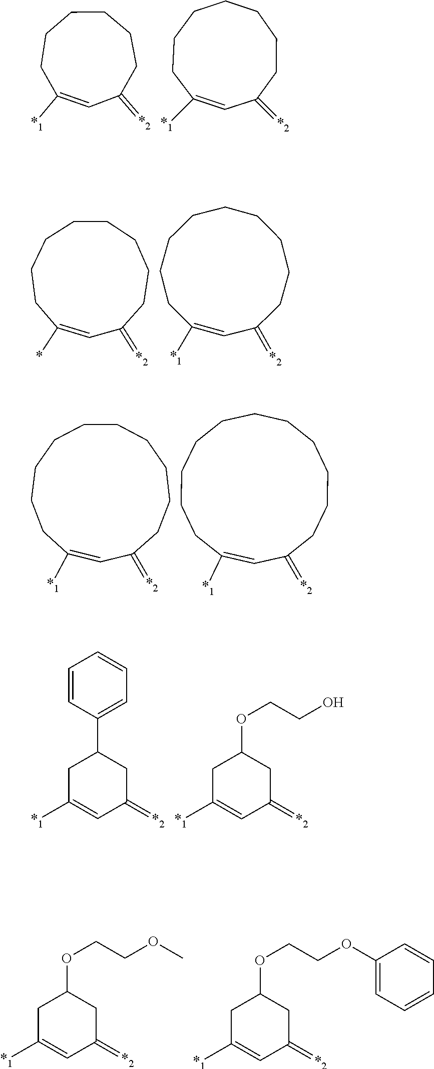

[in the formula, *1 represents a bond to a nitrogen atom, and *2 represents a bond to a carbon atom].

R¹ and R² are each independently preferably an alkyl group having 1 to 25 carbon atoms, more preferably an alkyl group having 1 to 12 carbon atoms, still more preferably a linear alkyl group having 1 to 6 carbon atoms, and particularly preferably a methyl group, an ethyl group, a n-propyl group, or a n-butyl group.

R⁶ and R⁷ are each independently preferably an alkyl group having 1 to 25 carbon atoms, more preferably an alkyl group having 1 to 12 carbon atoms, still more preferably a branched alkyl group having 1 to 6 carbon atoms, and particularly preferably an isopropyl group, an isobutyl group, or a t-butyl group.

R⁶ and R⁷ are preferably linked to each other to form a ring.

The compound (I) is preferably a compound represented by Formula (II).

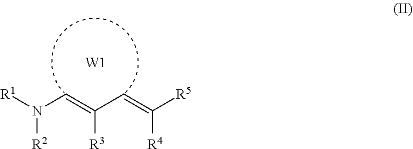

[in the formula, R¹, R², R³, R⁴, and R⁵ have the same meaning as described above; and

a ring W¹ represents a ring structure having at least one double bond as a constituent element of the ring and having no aromaticity].

Examples of the ring W¹ include the same ring as the ring formed by connecting R⁶ and R⁷ to each other.

The ring W¹ is preferably, for example, a ring described below.

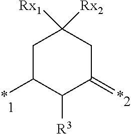

[in the formula, *1 represents a bond to a nitrogen atom, and *2 represents a bond to a carbon atom; and

Rₓ₁ and Rₓ₂ each independently represent a hydrogen atom or a substituent].

Examples of the substituents represented by Rₓ₁ and Rₓ₂ include the same substituents as that represented by R^{1E}.

The maximum absorption wavelength λ max of the compound (I) is preferably a wavelength of 370 nm or more and 420 nm or less. When the maximum absorption wavelength λ max of the compound (I) is a wavelength of 370 nm or more and 420 nm or less, ultraviolet to near-ultraviolet light having a wavelength in a range of 380 nm or more and 400 nm or less can be efficiently absorbed. λ max may have a wavelength of more than 370 nm and 420 nm or less, preferably a wavelength of 375 nm or more and 415 nm or less, more preferably a wavelength of 375 nm or more and 410 nm or less, and still more preferably a wavelength of 380 nm or more and 400 nm or less.

The gram absorption coefficient ε at λ max of the compound (I) is preferably 0.5 or more, more preferably 0.75 or more, and particularly preferably 1.0 or more. The upper limit is not particularly limited, and is generally 10 or less. λ max represents the maximum absorption wavelength [nm] of the compound (I).

When the gram absorption coefficient ε at λ max of the compound (I) is 0.5 or more, ultraviolet to near-ultraviolet light in a wavelength range of 370 nm or more and 420 nm or less can be efficiently absorbed even with a small addition amount.

In the compound (I), ε (λ max)/ε (λ max+30 nm) is preferably 5 or more, more preferably 10 or more, and particularly preferably 20 or more. The upper limit is not particularly limited, and is generally 1000 or less. ε (λ max) represents a gram absorption coefficient at a maximum absorption wavelength [nm] of the compound (I), and ε (λ max+30 nm) represents a gram absorption coefficient at a wavelength [nm] of (maximum absorption wavelength [nm]+30 nm) of the compound (I).

When ε (λ max)/ε (λ max+30 nm) is 5 or more, secondary absorption at a wavelength of 420 nm or more can be minimized, so that coloring is less likely to occur.

The unit of the gram absorption coefficient is L/(g·cm).

Examples of the compound (I) include the compounds described below.

The compound (I) is preferably compounds represented by Formula (1-1) to Formula (1-4), Formula (1-7), Formula (1-8), Formula (1-10), Formula (1-12), Formula (1-20) to Formula (1-25), Formula (1-55) to Formula (1-57), Formula (1-59), Formula (1-63), Formula (1-64), Formula (1-67), Formula (1-69) to Formula (1-76), Formula (1-123), Formula (1-126), Formula (1-127), Formula (1-129), Formula (1-161), Formula (1-166), and Formula (1-337) to Formula (1-404),

is more preferably compounds represented by Formula (1-4), Formula (1-7), Formula (1-20), Formula (1-55), Formula (1-56), Formula (1-59), Formula (1-63), Formula (1-64), Formula (1-123), Formula (1-126), Formula (1-127), Formula (1-129), Formula (1-141), Formula (1-147), Formula (1-153), and Formula (1-337) to Formula (1-404), and

is still more preferably compounds represented by Formula (1-55), Formula (1-59), Formula (1-63), Formula (1-64), Formula (1-123), Formula (1-141), Formula (1-147), Formula (1-153), and Formula (1-337) to Formula (1-404).

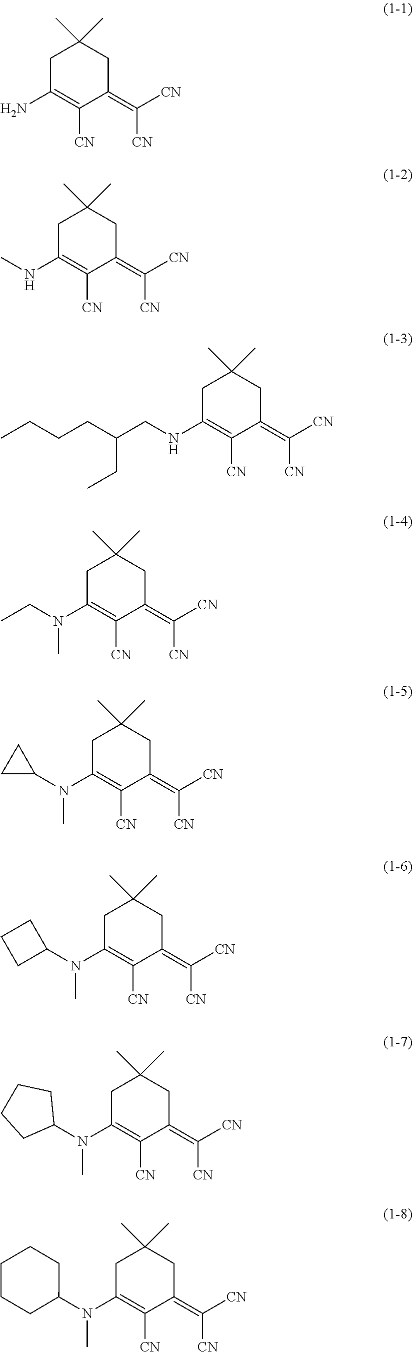

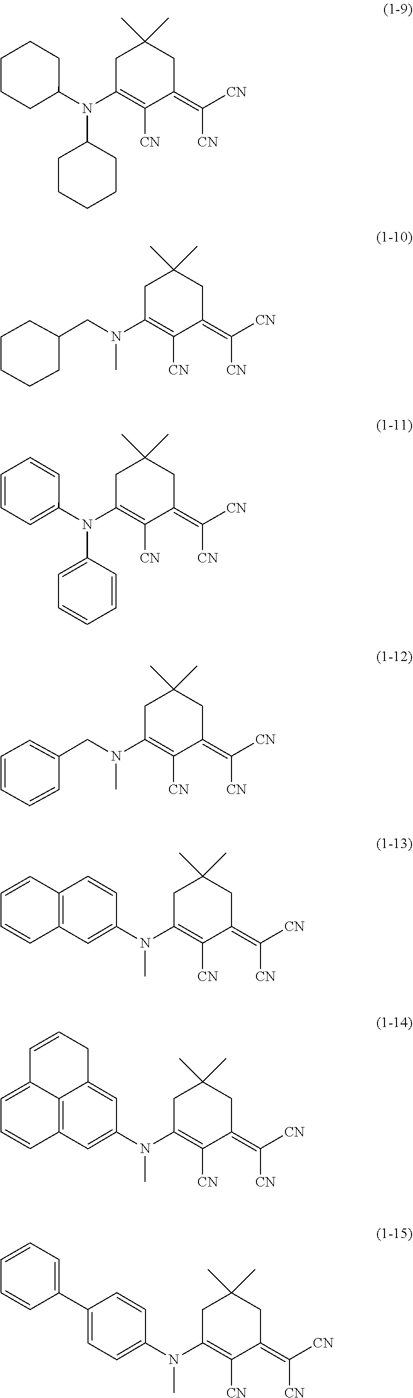

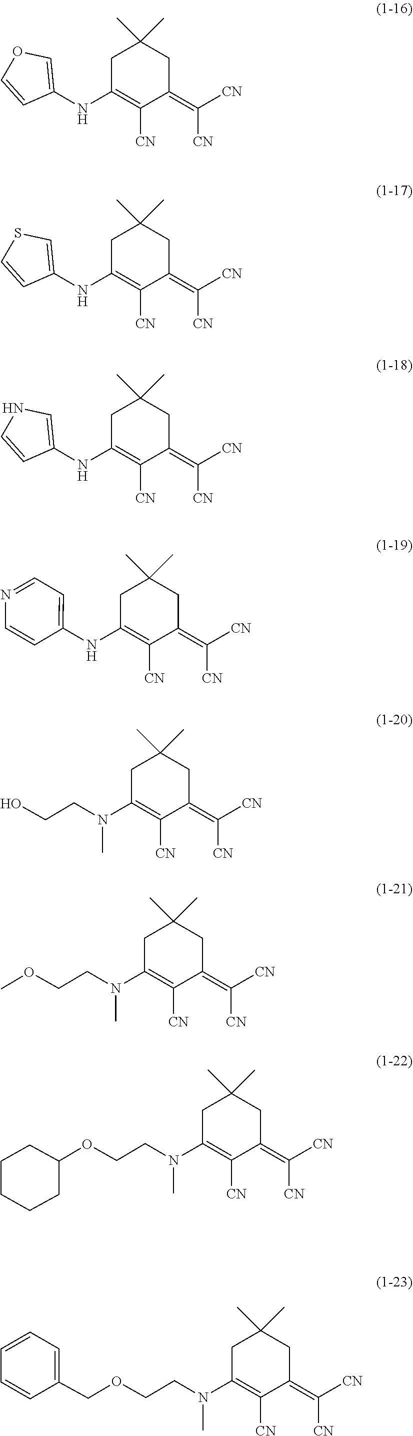

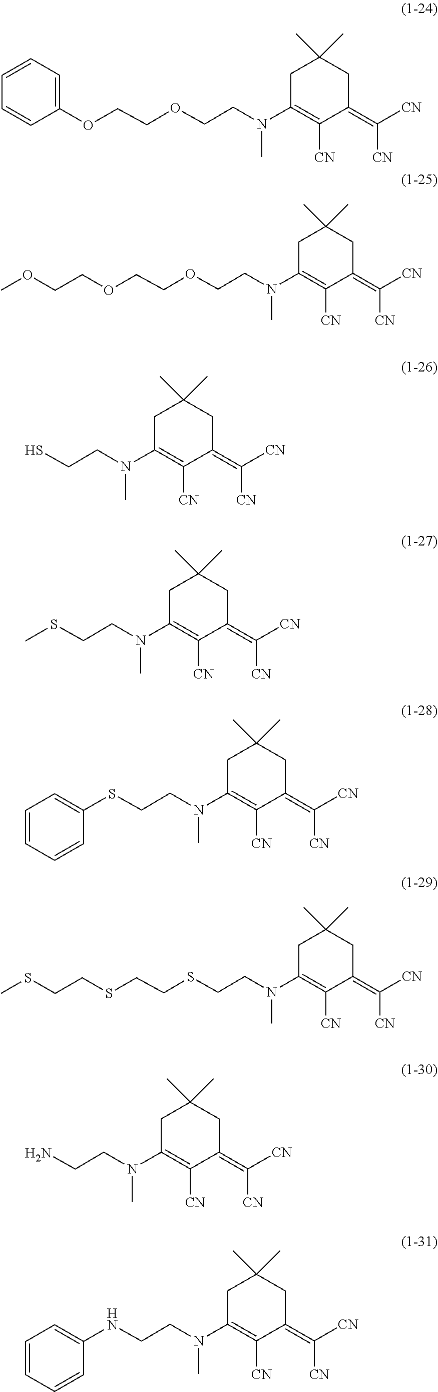

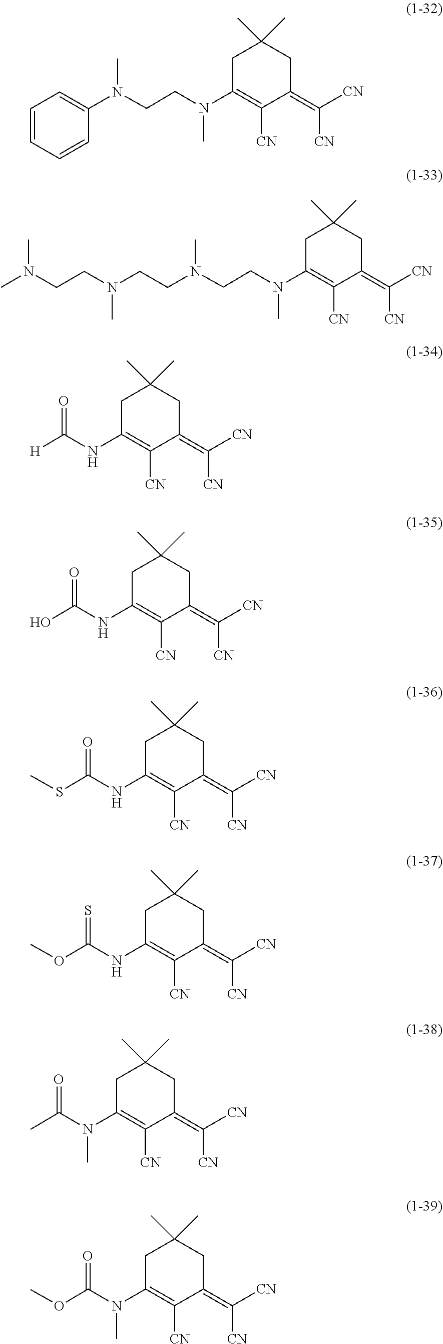

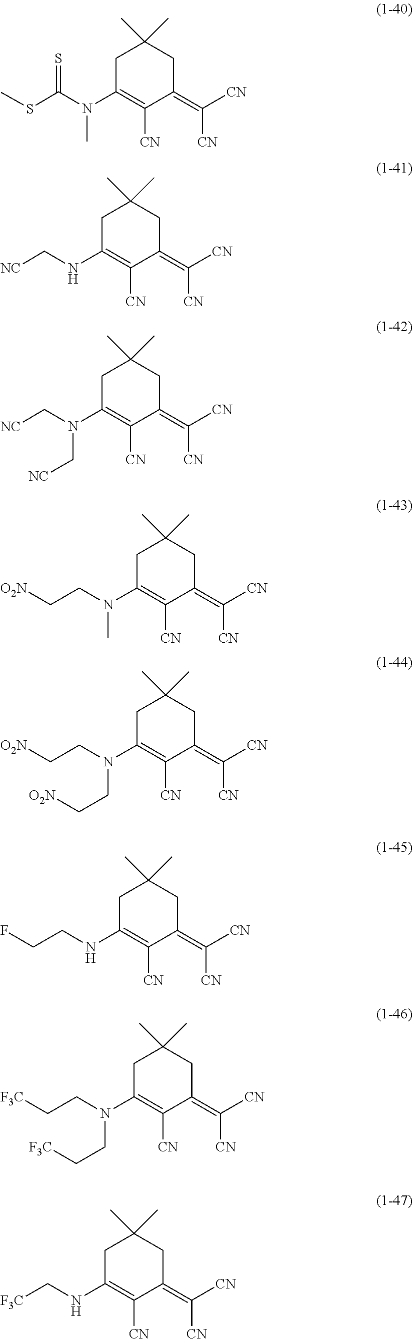

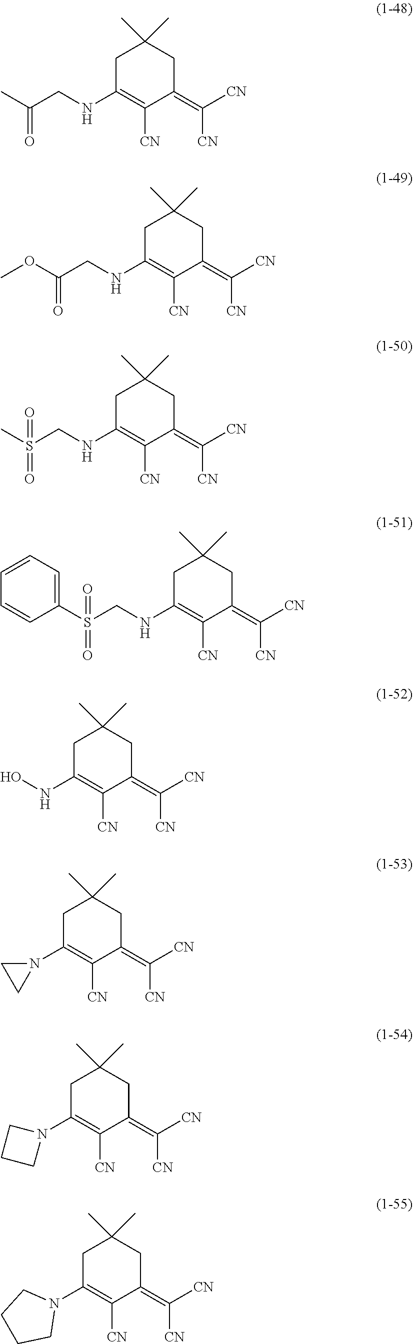

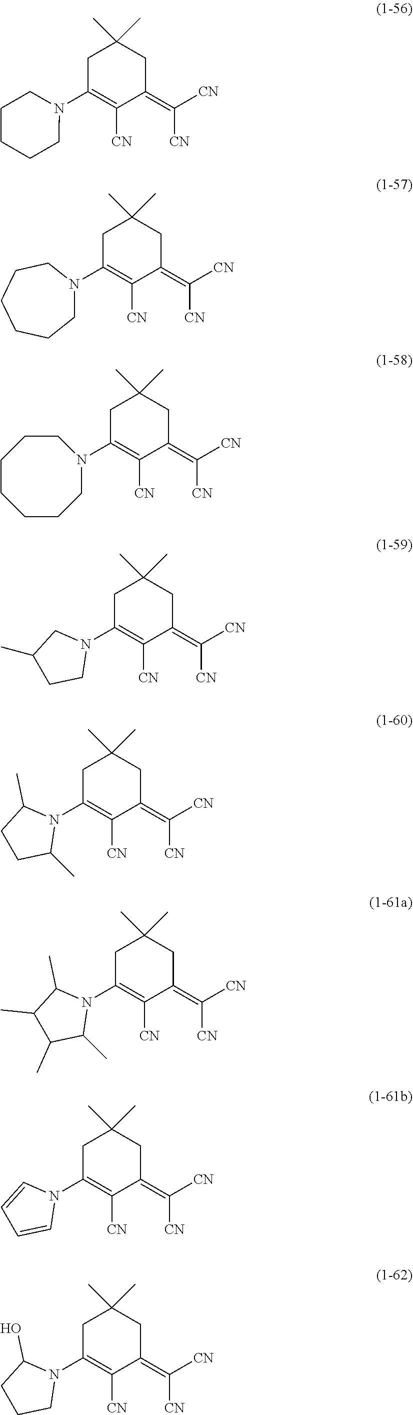

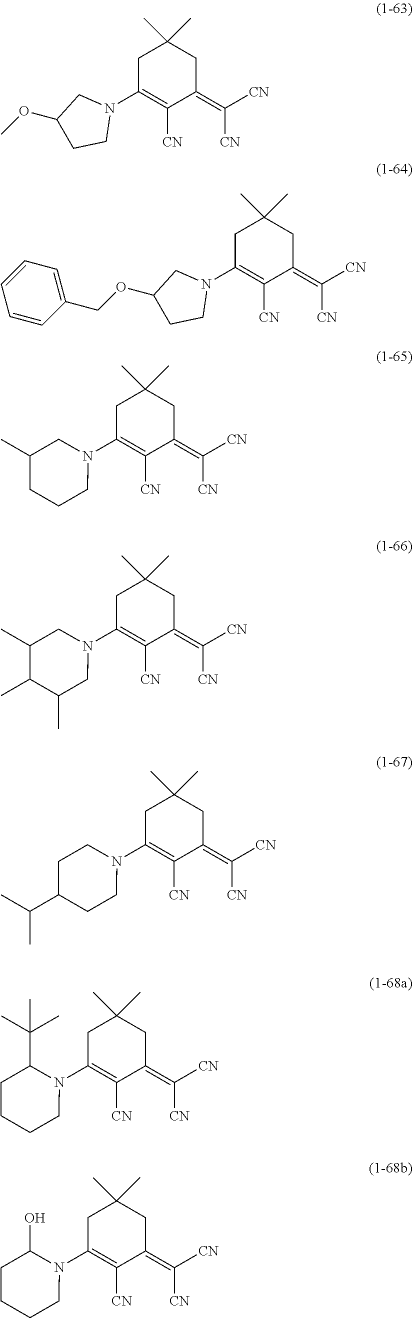

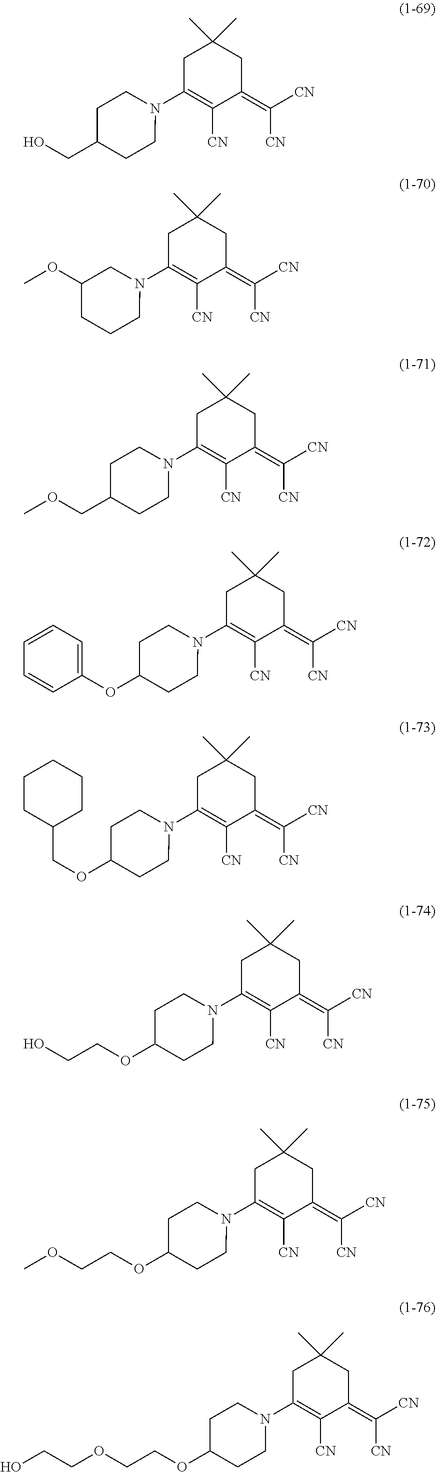

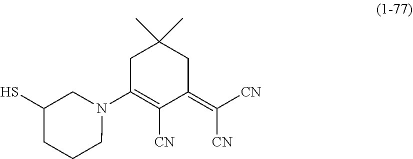 [SMILES: CC1(C)CC(=C(C#N)C#N)C(C#N)=C(N2CCCC(S)C2)C1]

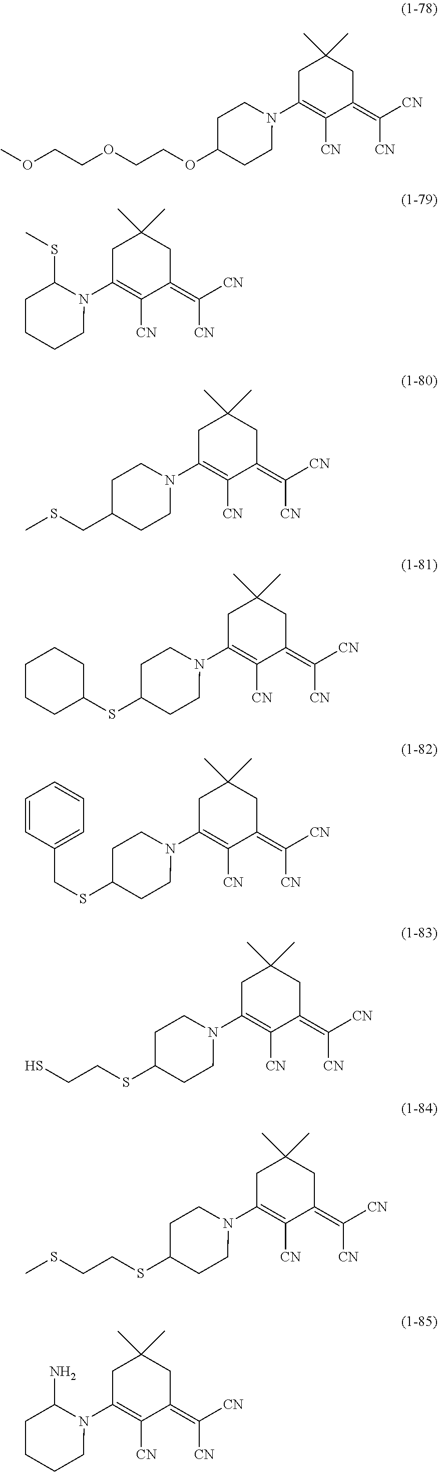

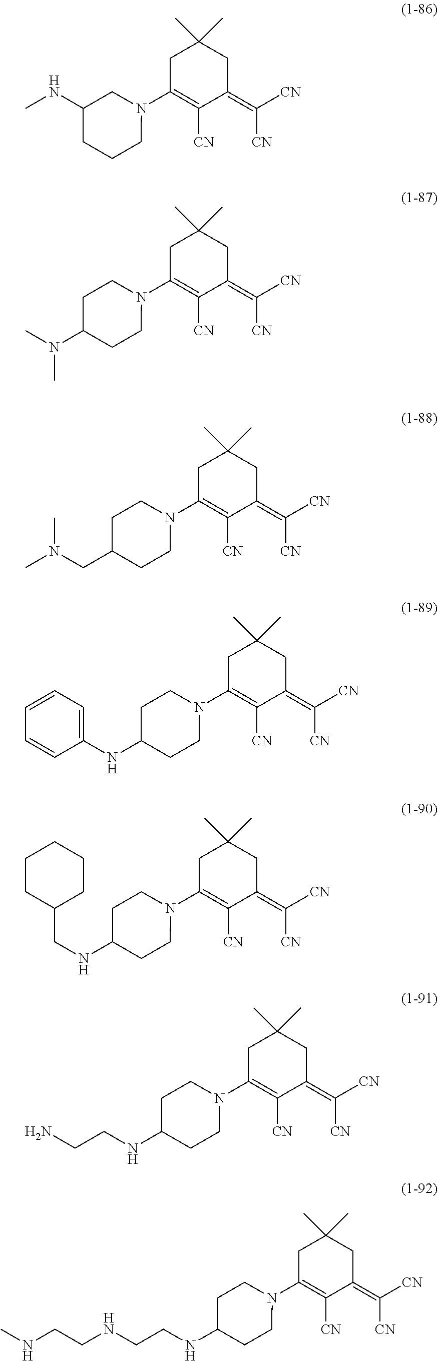

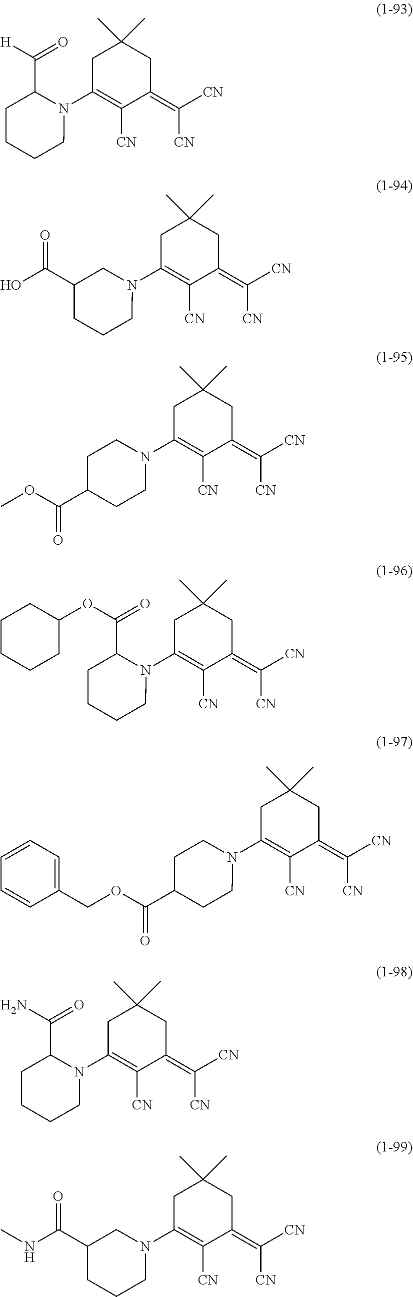

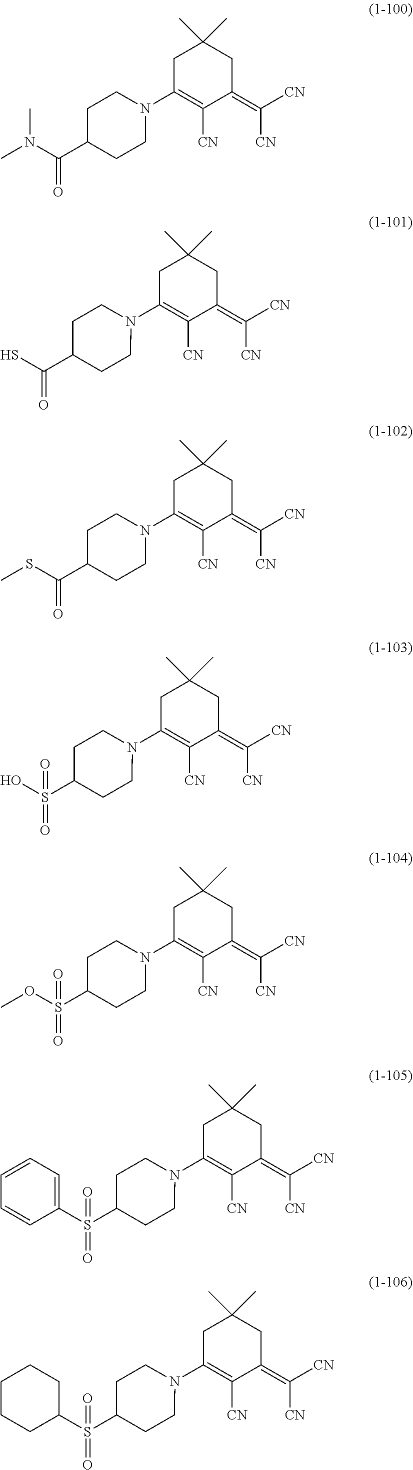

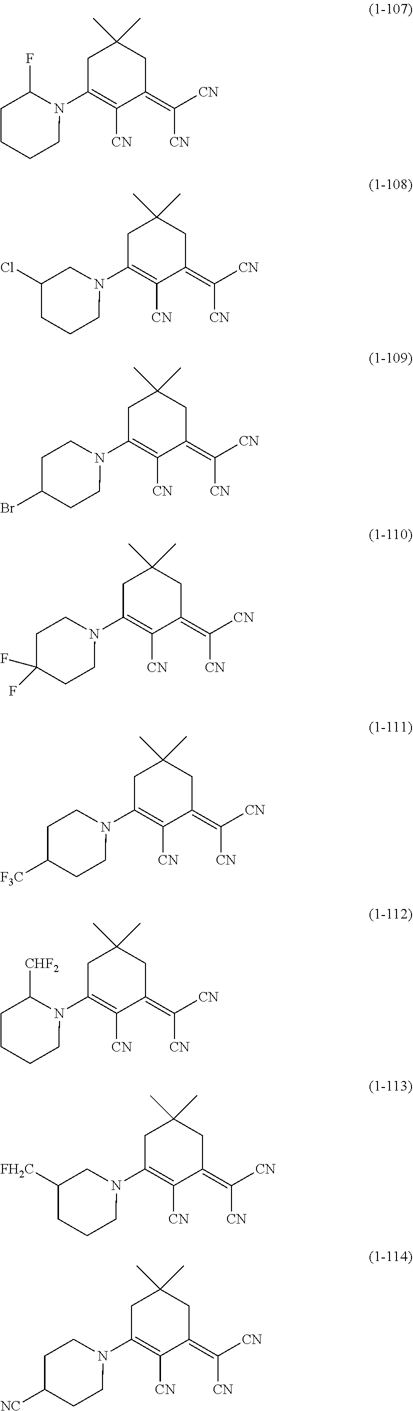

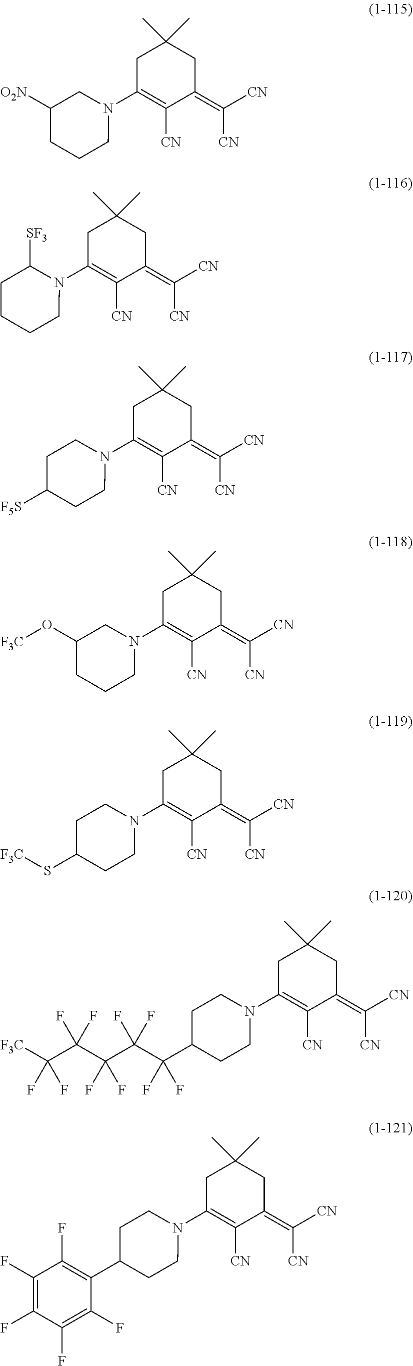

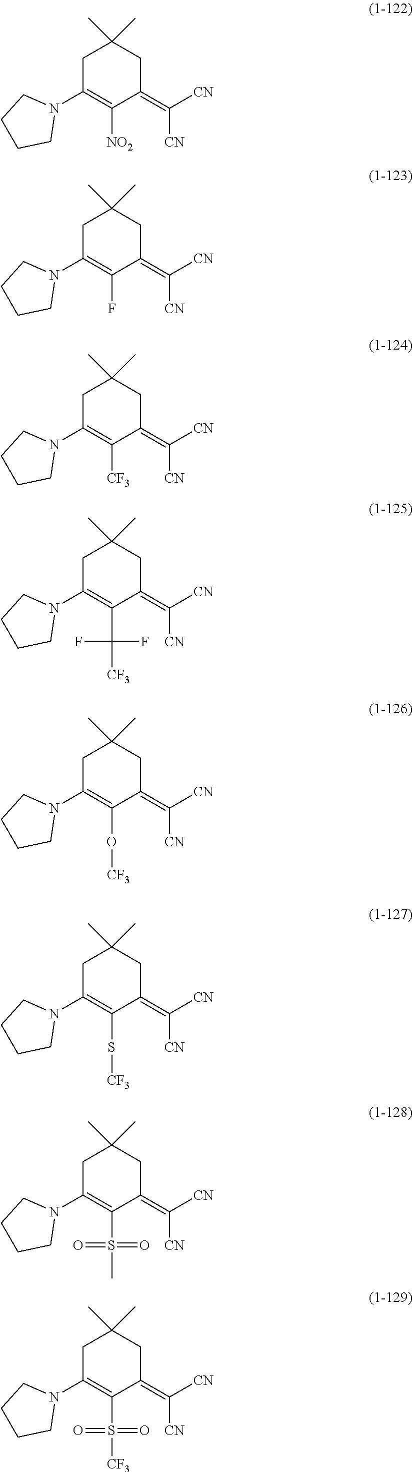

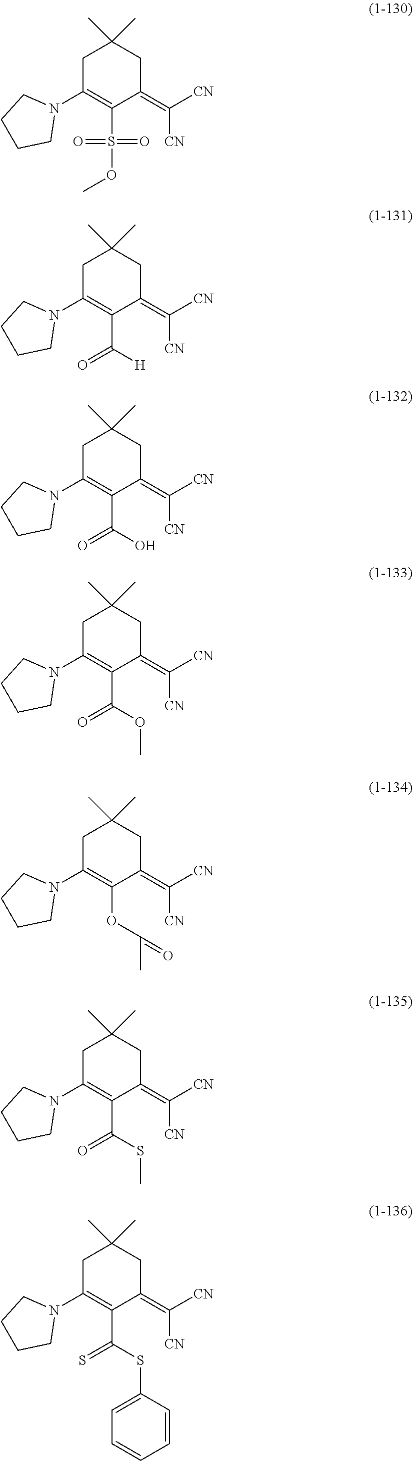

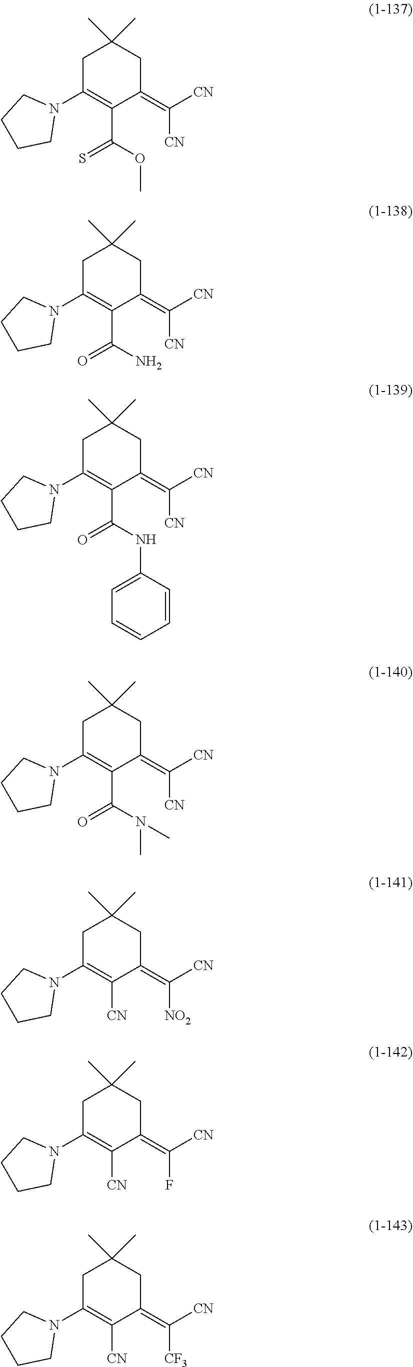

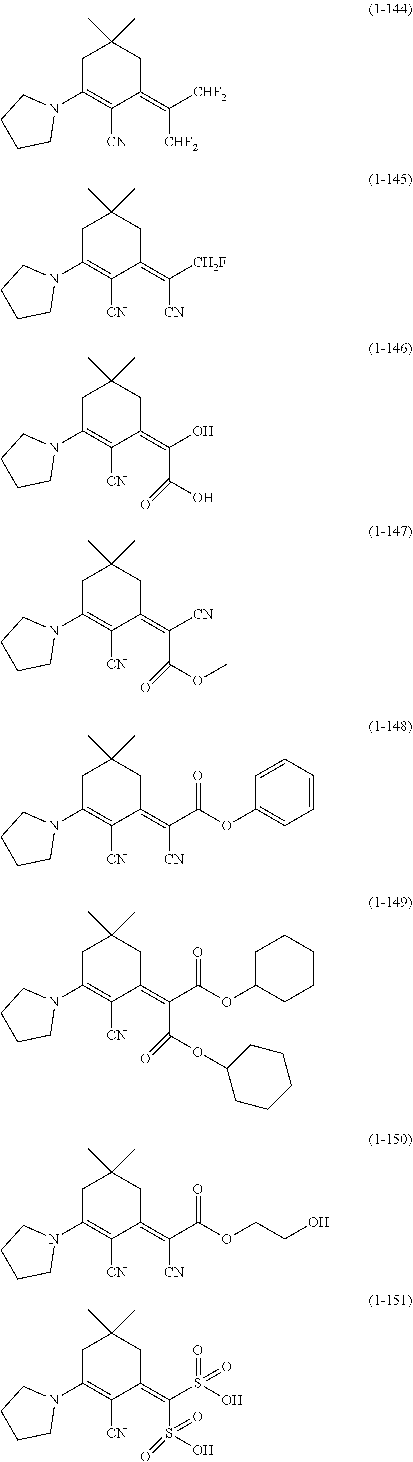

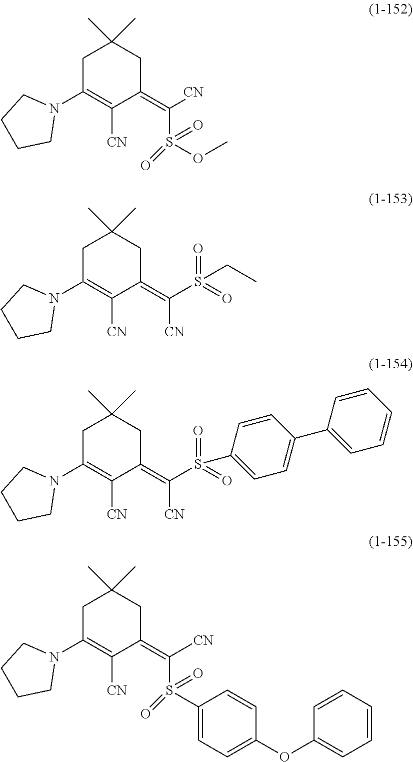

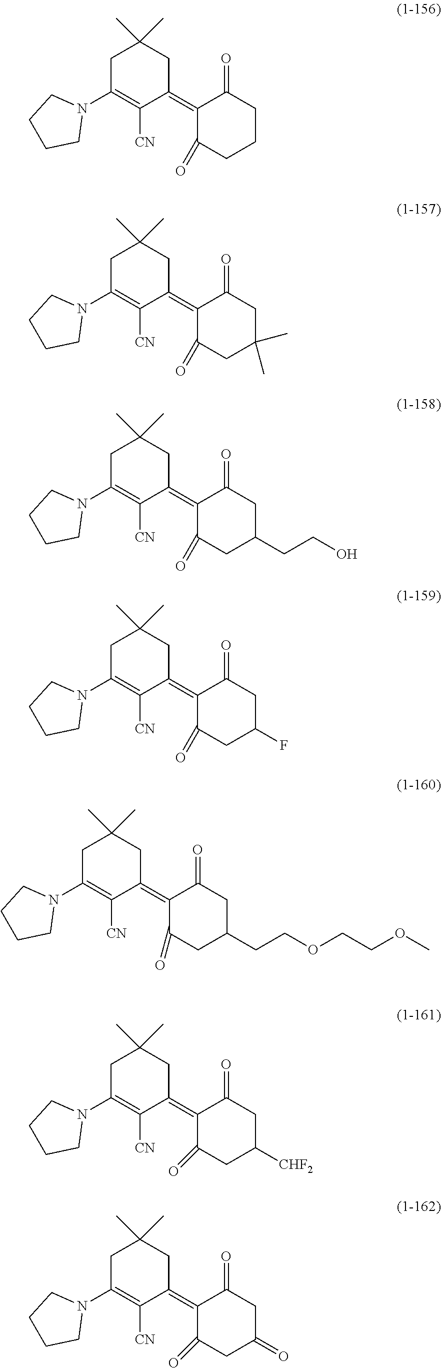

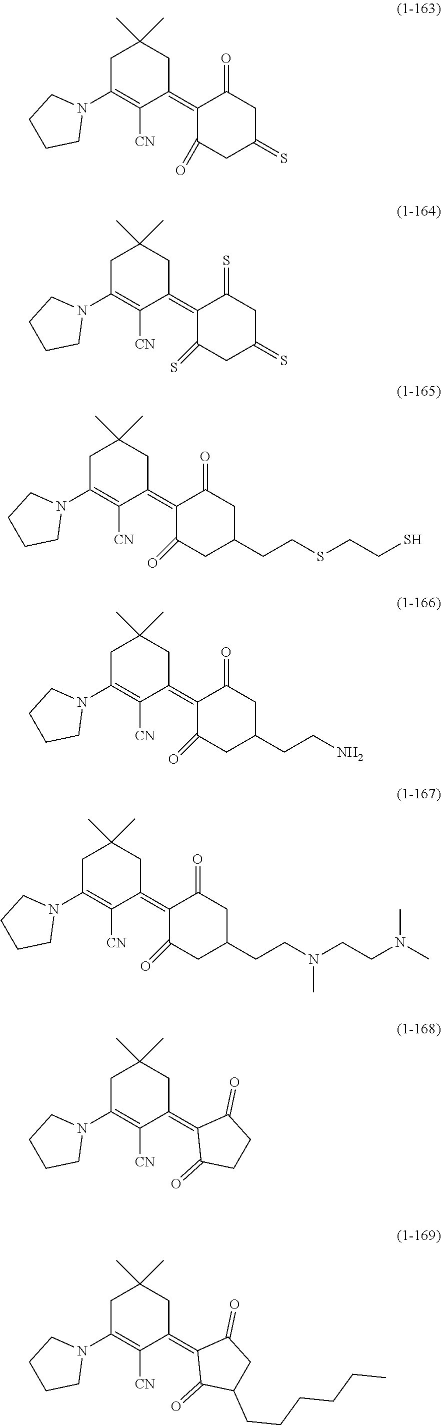

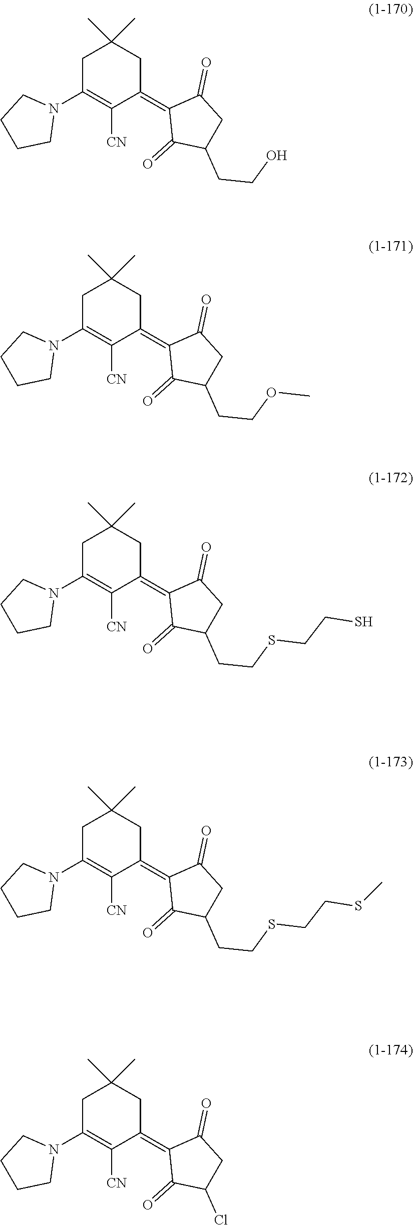 [SMILES: CC1(C)CC(N2CCCC2)=C(C#N)/C(=C2/C(=O)CC(CCO)C2=O)C1 ; COCCC1CC(=O)/C(=C2\CC(C)(C)CC(N3CCCC3)=C2C#N)C1=O ; CC1(C)CC(N2CCCC2)=C(C#N)/C(=C2/C(=O)CC(CCSCCS)C2=O)C1 ; CSCCSCCC1CC(=O)/C(=C2\CC(C)(C)CC(N3CCCC3)=C2C#N)C1=O ; CC1(C)CC(N2CCCC2)=C(C#N)/C(=C2/C(=O)CC(Cl)C2=O)C1]

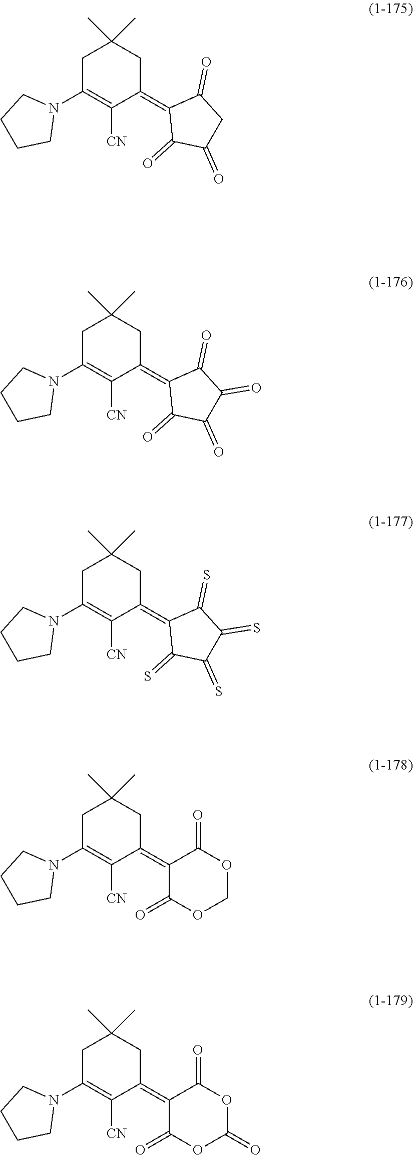 [SMILES: CC1(C)CC(N2CCCC2)=C(C#N)/C(=C2/C(=O)CC(=O)C2=O)C1 ; CC1(C)CC(=C2C(=O)C(=O)C(=O)C2=O)C(C#N)=C(N2CCCC2)C1 ; CC1(C)CC(=C2C(=S)C(=S)C(=S)C2=S)C(C#N)=C(N2CCCC2)C1 ; CC1(C)CC(=C2C(=O)OCOC2=O)C(C#N)=C(N2CCCC2)C1 ; CC1(C)CC(=C2C(=O)OC(=O)OC2=O)C(C#N)=C(N2CCCC2)C1]

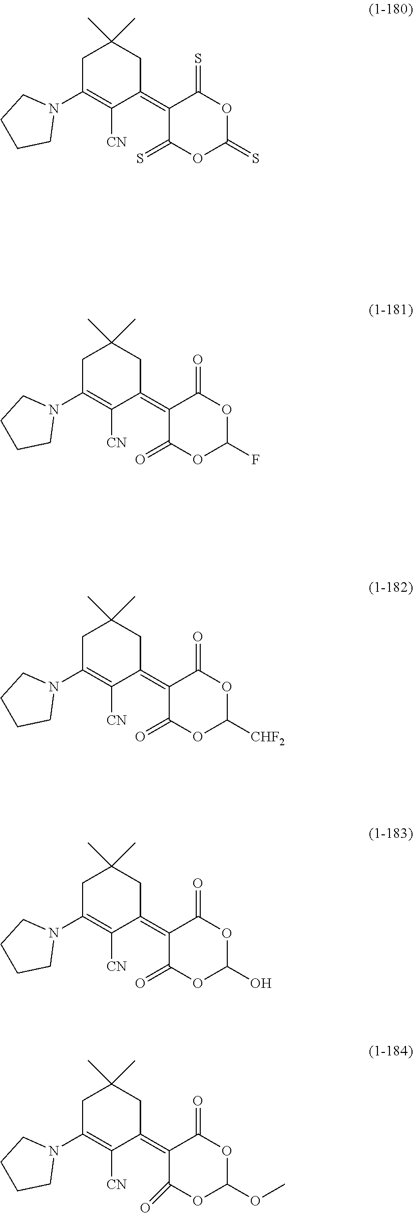 [SMILES: CC1(C)CC(=C2C(=S)OC(=S)OC2=S)C(C#N)=C(N2CCCC2)C1 ; CC1(C)CC(=C2C(=O)OC(F)OC2=O)C(C#N)=C(N2CCCC2)C1 ; CC1(C)CC(=C2C(=O)OC(C(F)F)OC2=O)C(C#N)=C(N2CCCC2)C1 ; CC1(C)CC(=C2C(=O)OC(O)OC2=O)C(C#N)=C(N2CCCC2)C1 ; COC1OC(=O)C(=C2CC(C)(C)CC(N3CCCC3)=C2C#N)C(=O)O1]

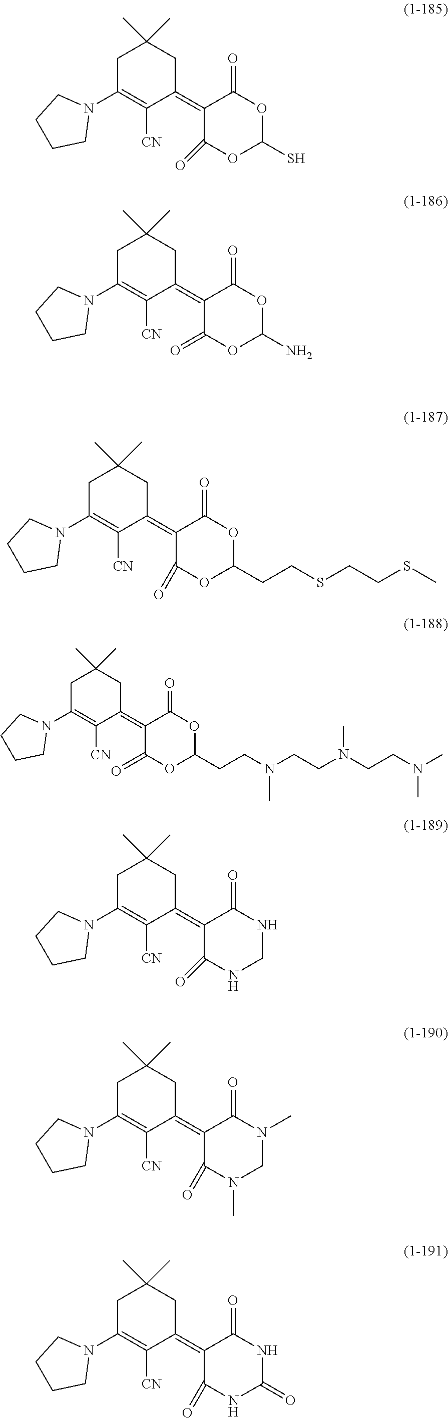

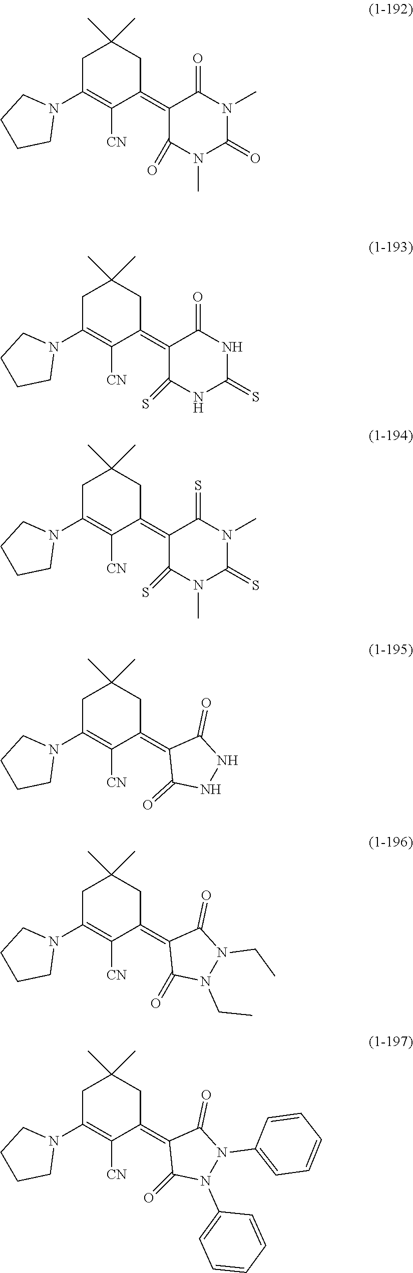

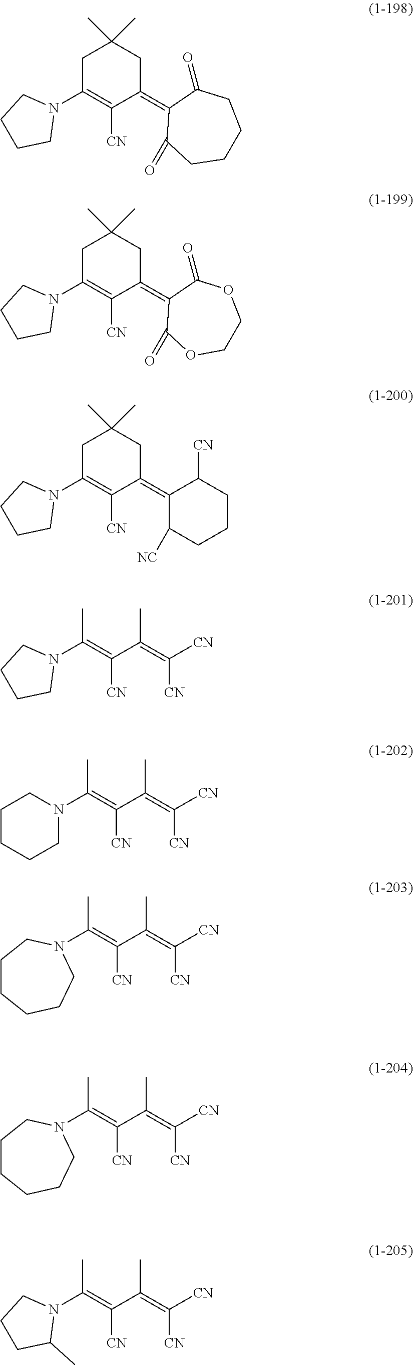

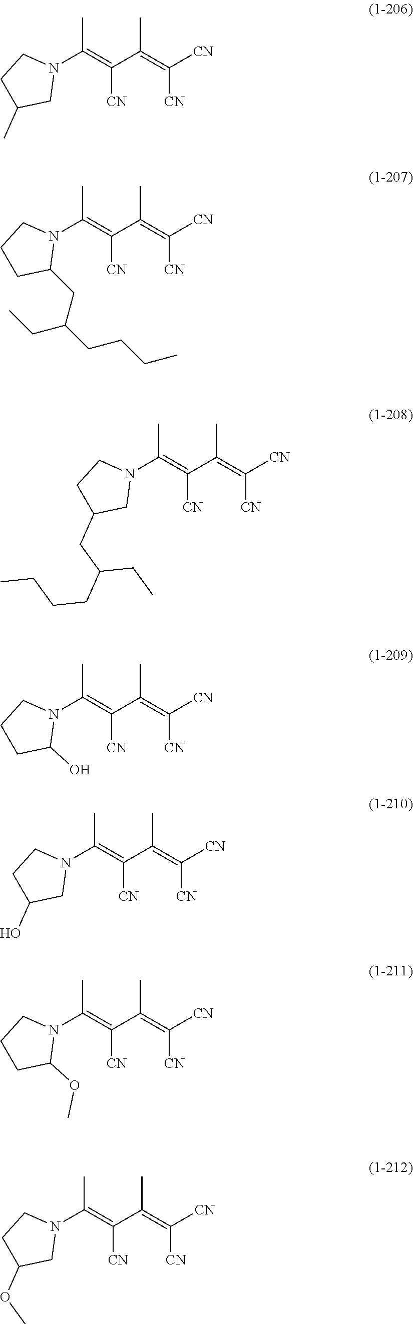

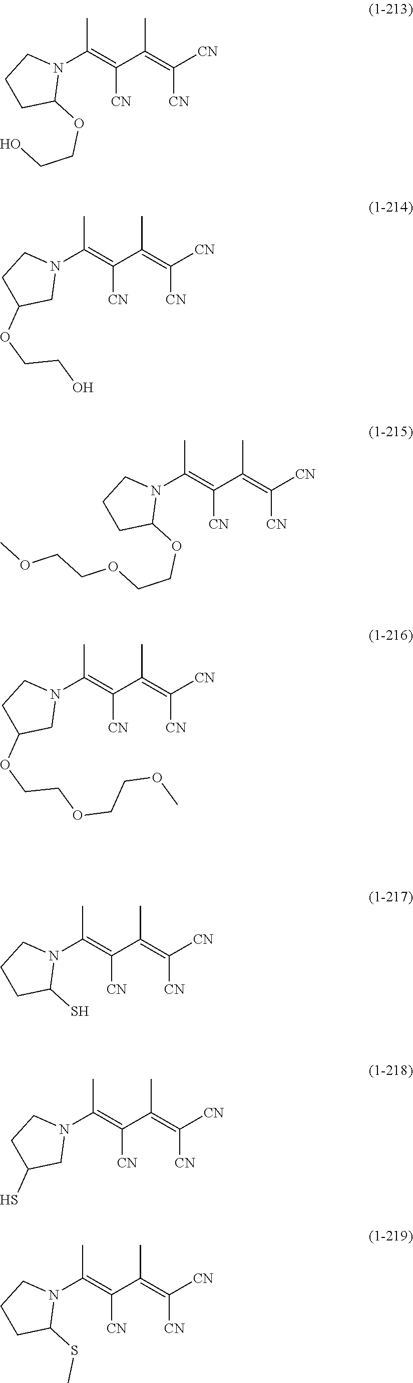

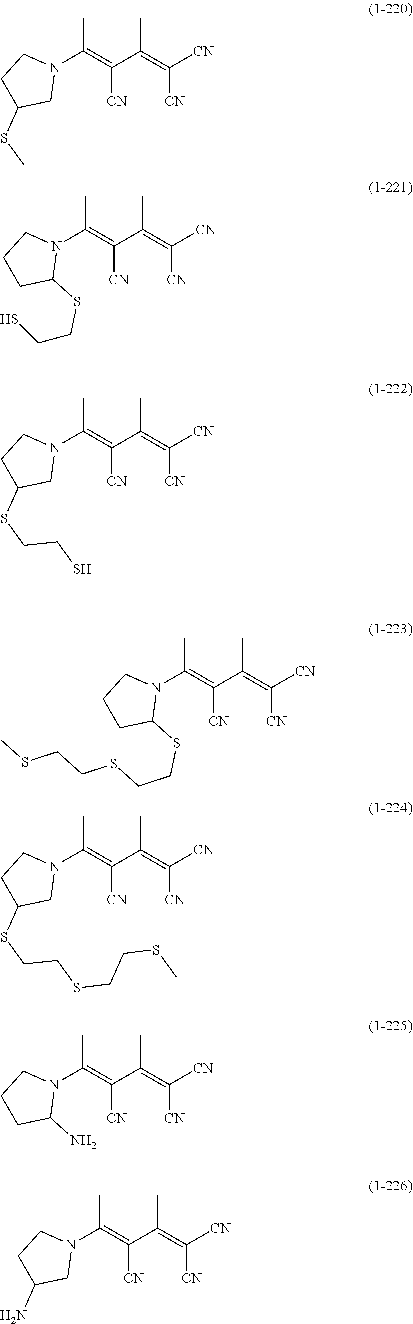

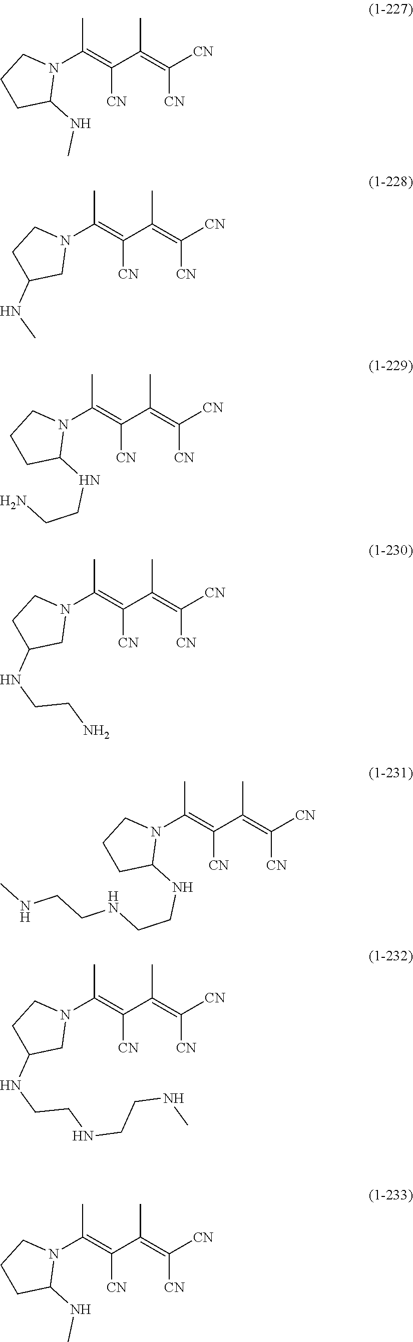

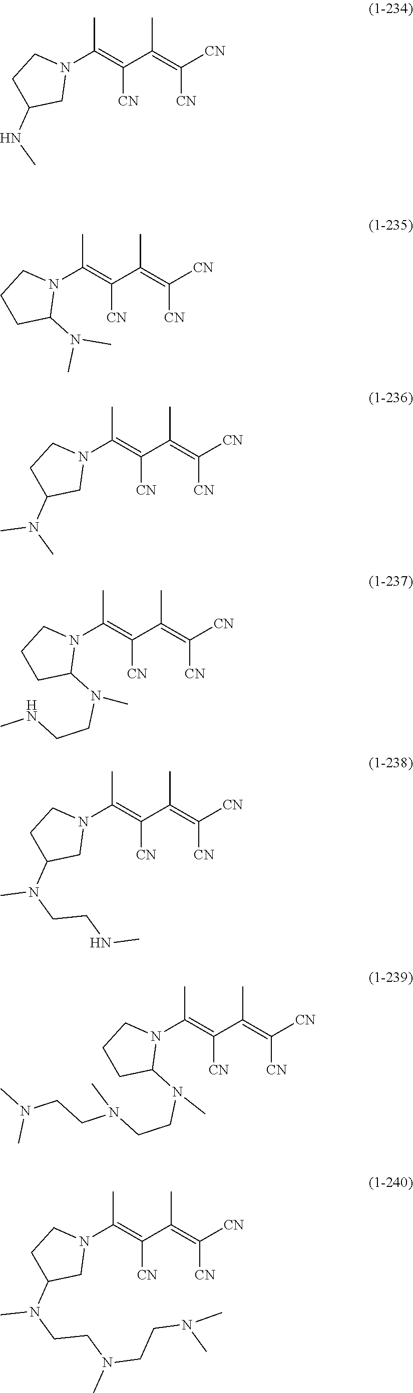

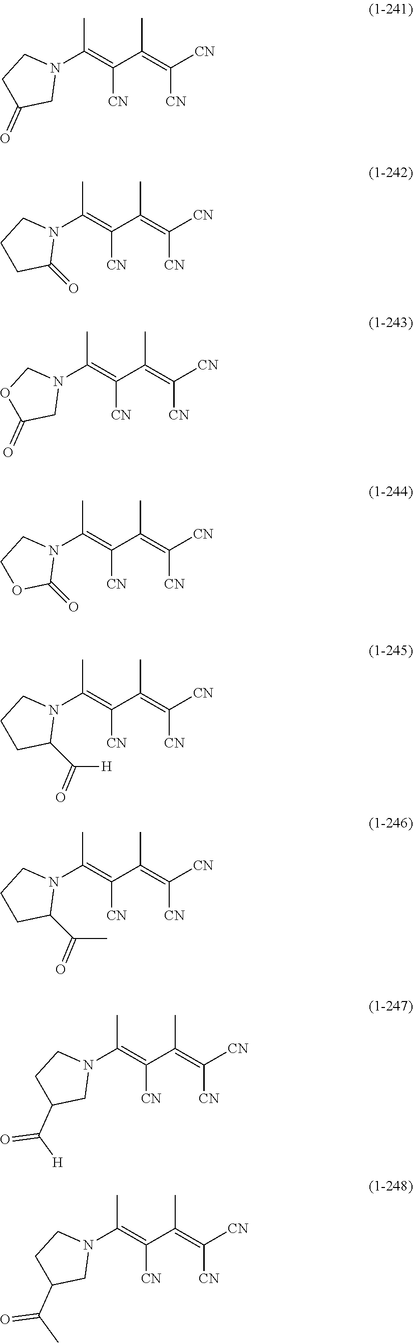

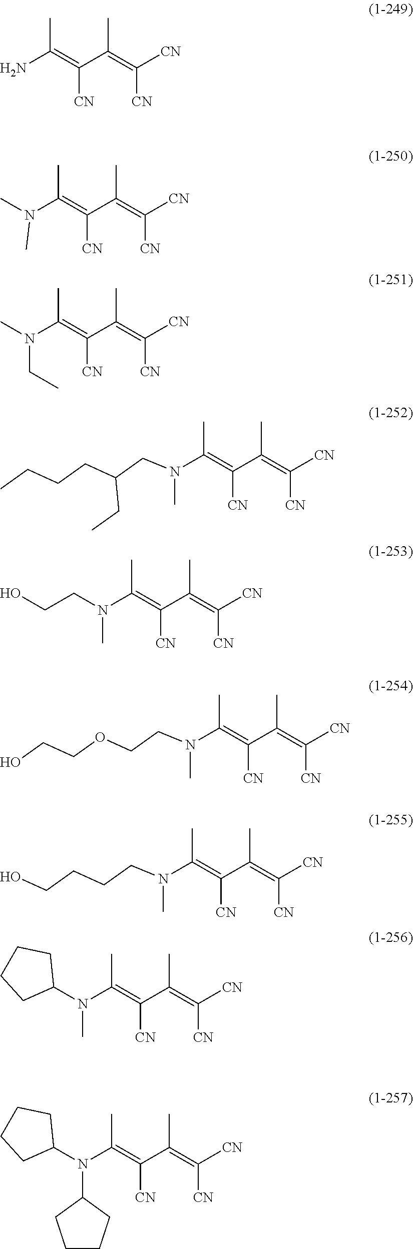

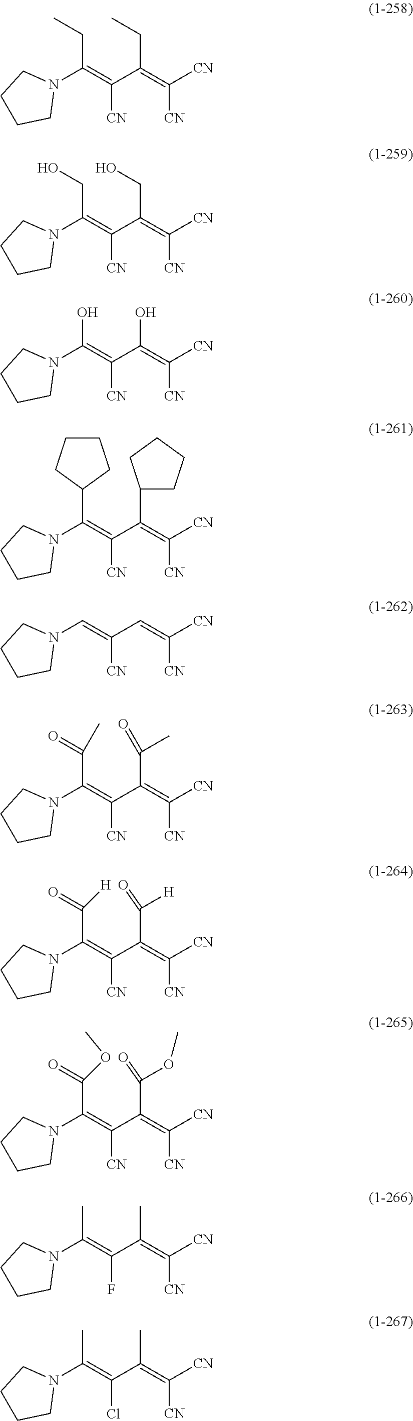

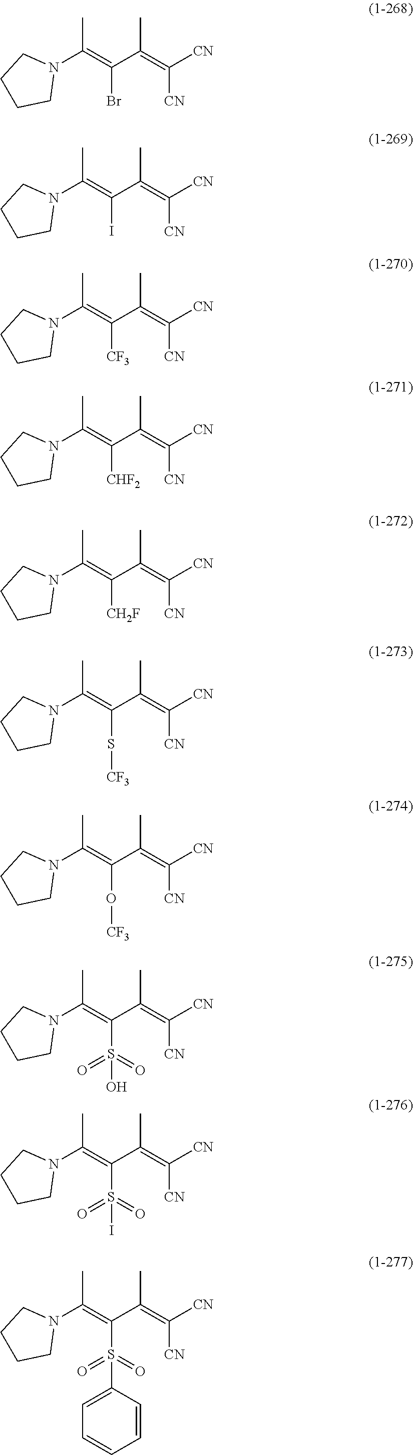

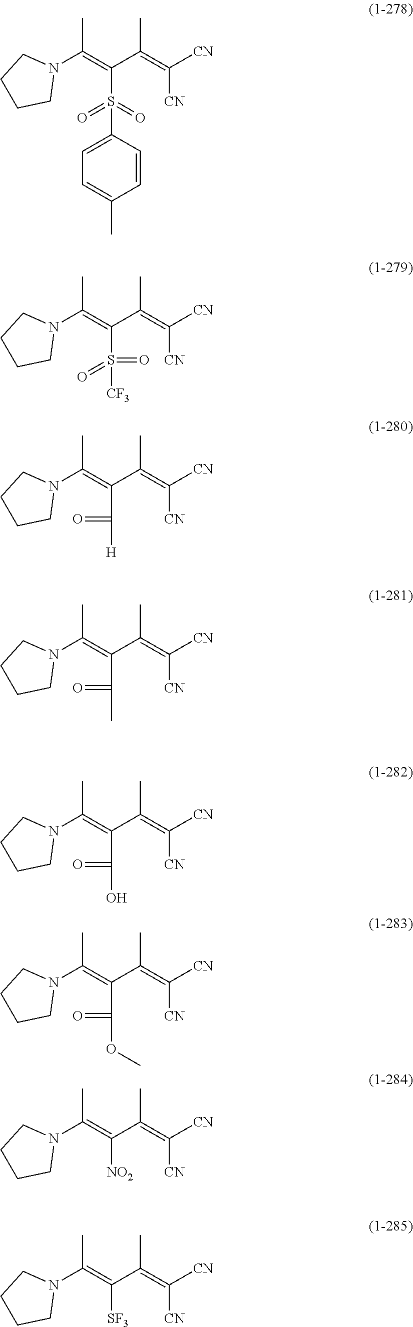

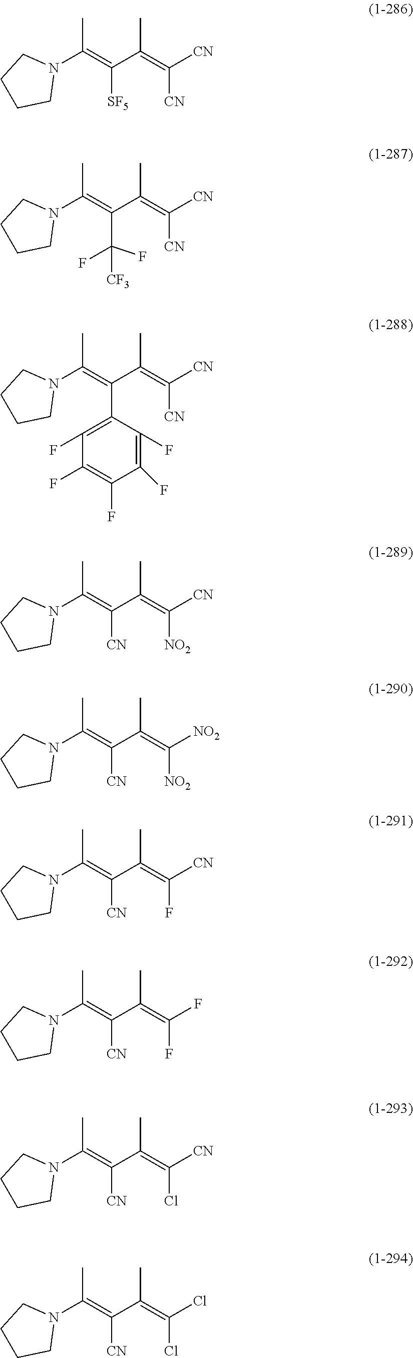

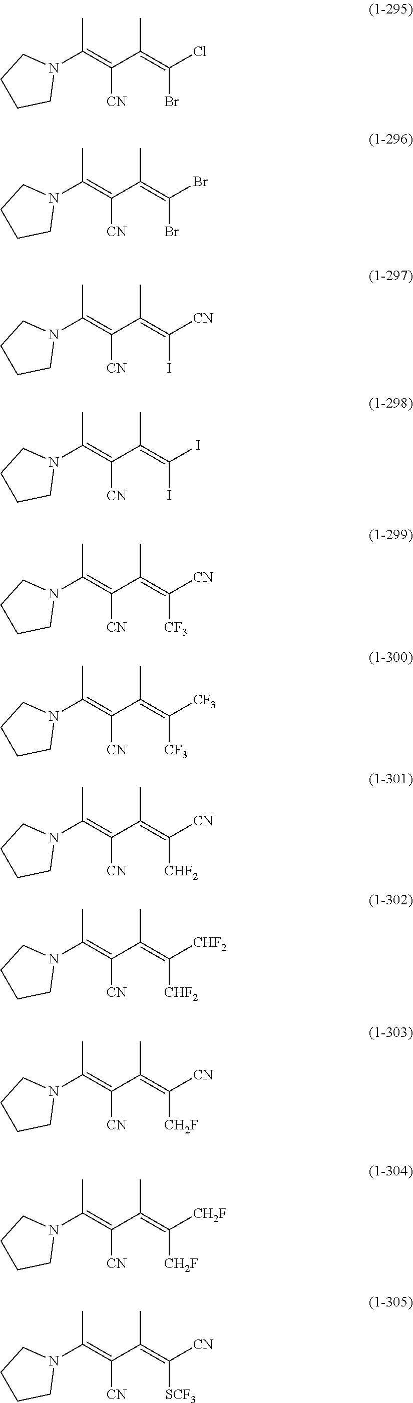

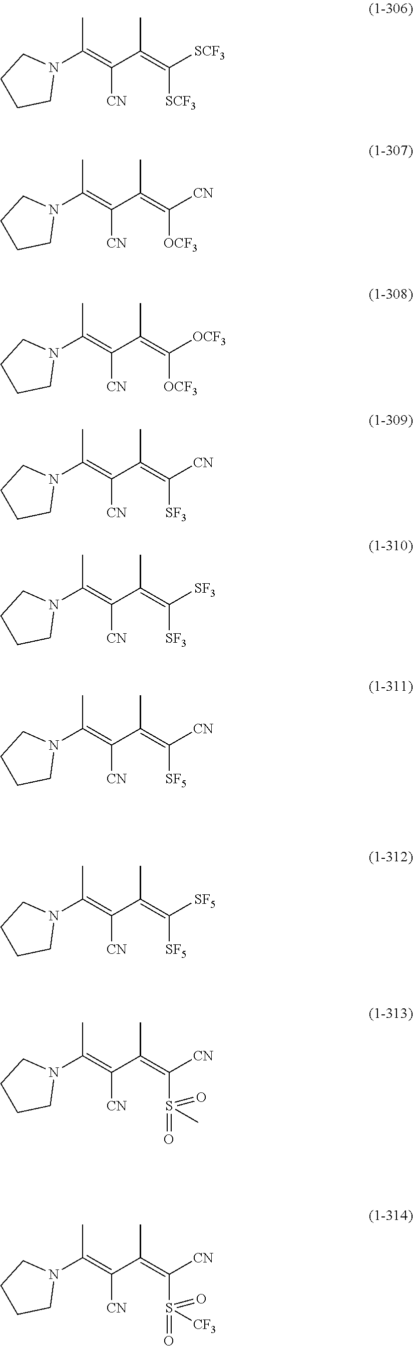

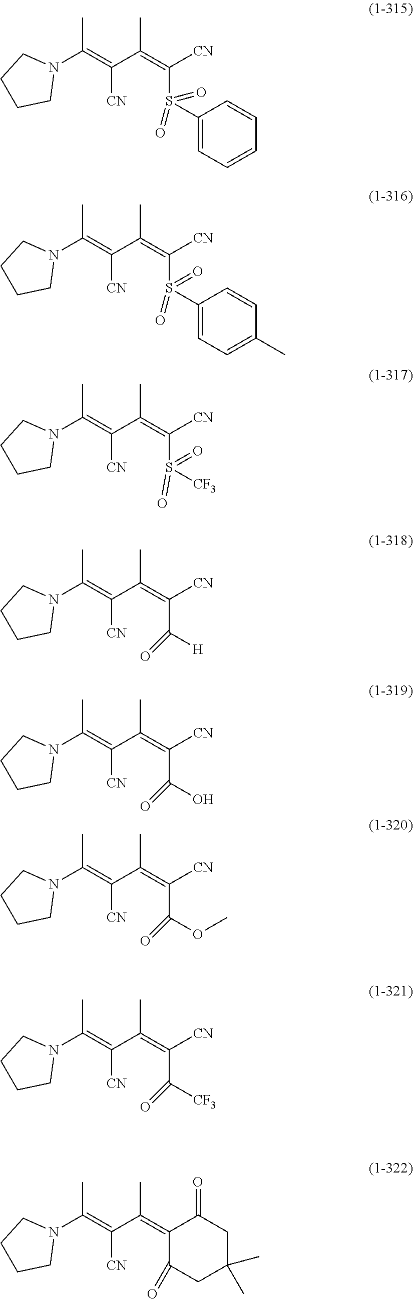

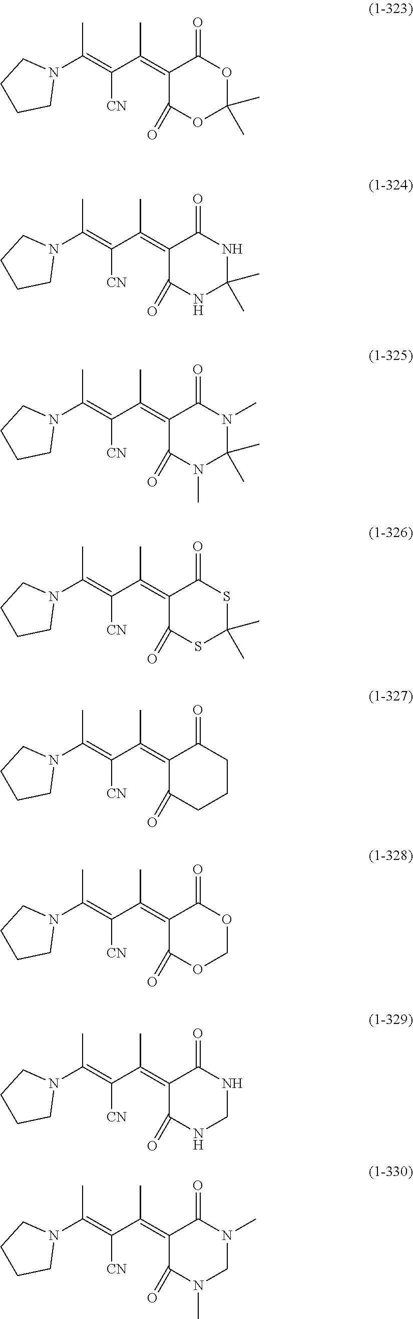

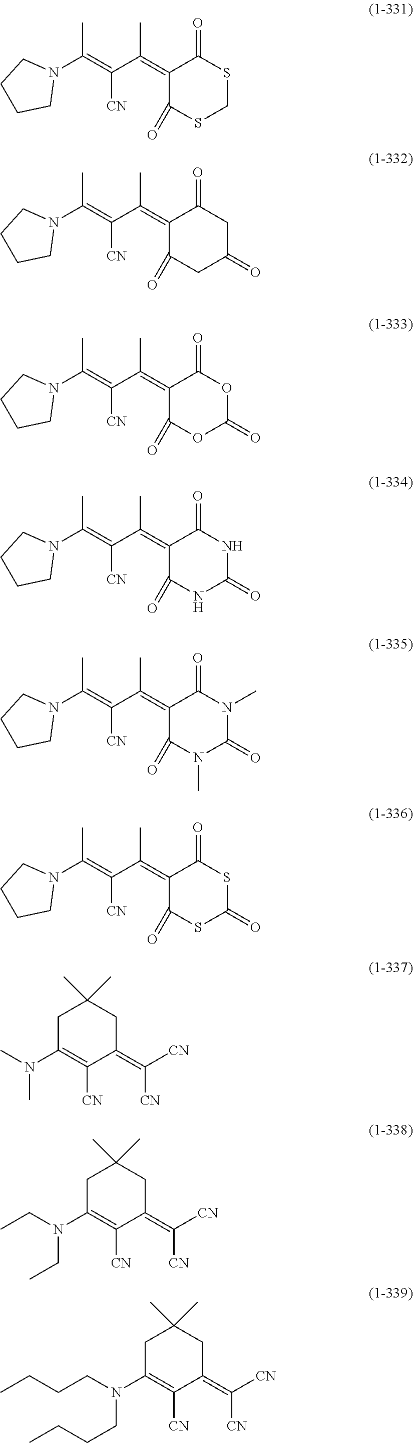

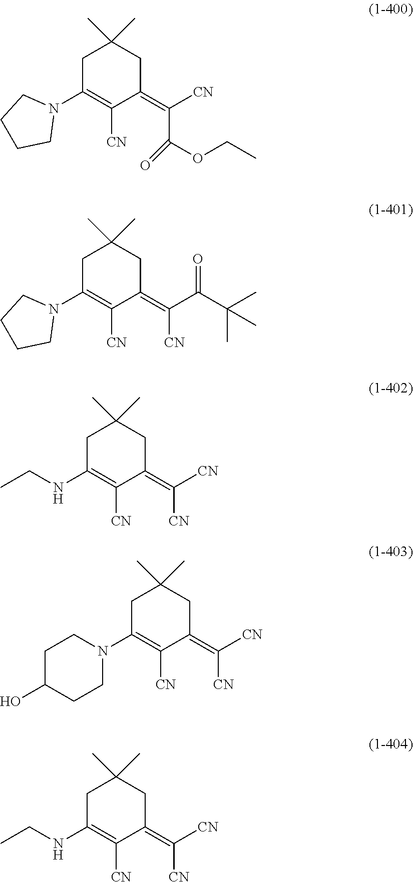

<Method for Producing Compound (I)>

The compound (I) can be obtained, for example, by reacting a compound represented by Formula (I-1) (hereinafter, may be referred to as a compound (I-1)) with a compound represented by Formula (I-2) (hereinafter, may be referred to as a compound represented by Formula (I-2)).

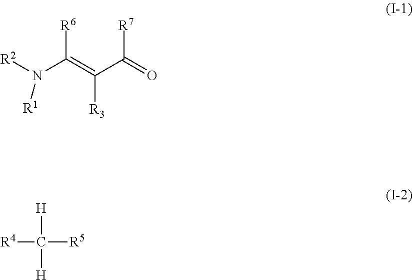

[in the formula, R¹ to R⁷ have the same meaning as described above].

The reaction between the compound (I-1) and the compound (I-2) is usually performed by mixing the compound (I-1) and the compound (I-2), and it is preferable to add the compound (I-2) to the compound (I-1).

In addition, as for the reaction between the compound (I-1) and the compound (I-2), it is preferable to mix the compound (I-1) and the compound (I-2) in the presence of a base and a methylating agent,

it is preferable to mix the compound (1-1), the compound (I-2), a base, and a methylating agent,

it is more preferable to mix the compound (I-2) and a base in a mixture of the compound (1-1) and a methylating agent, and

it is still more preferable to add a mixture of the compound (I-2) and a base to the mixture of the compound (1-1) and the methylating agent.

Examples of the base include metal hydroxides (preferably alkali metal hydroxides) such as sodium hydroxide, lithium hydroxide, potassium hydroxide, cesium hydroxide, rubidium hydroxide, calcium hydroxide, barium hydroxide, and magnesium hydroxide; metal alkoxides (preferably alkali metal alkoxides) such as sodium methoxide, potassium methoxide, lithium methoxide, sodium ethoxide, sodium isopropoxide, sodium tertiary butoxide, and potassium tertiary butoxide; metal hydrides such as lithium hydride, sodium hydride, potassium hydride, lithium aluminum hydride, sodium borohydride, aluminum hydride, and sodium aluminum hydride; metal oxides such as calcium oxide and magnesium oxide; metal carbonates (preferably alkaline earth metal carbonates) such as sodium hydrogen carbonate, sodium carbonate, and potassium carbonate; organic alkyl metal compounds such as n-butyl lithium, tertiary butyl lithium, methyl lithium, and Grignard reagent; amine compounds (preferably tertiary amines such as triethylamine and diisopropylethylamine) such as ammonia, triethylamine, diisopropylethylamine, ethanolamine, pyrrolidine, piperidine, diazabicycloundecene, diazabicyclononene, guanidine, tetramethylammonium hydroxide, tetraethylammonium hydroxide, pyridine, aniline, dimethoxyaniline, ammonium acetate, and β-alanine; metal amide compounds (preferably alkali metal amides) such as lithium diisopropylamide, sodium amide, and potassium hexamethyldisilazide; sulfonium compounds such as trimethylsulfonium hydroxide; iodonium compounds such as diphenyliodonium hydroxide; and phosphazene bases.

The use amount of the base is usually 0.1 to 5 mol and preferably 0.5 to 2 mol, with respect to 1 mol of the compound (I-1).

Examples of the methylating agent include iodomethane, dimethyl sulfate, methyl methanesulfonate, methyl fluorosulfonate, methyl p-toluenesulfonate, methyl trifluoromethanesulfonate, and trimethyloxonium tetrafluoroborate.

The use amount of the methylating agent is usually 0.1 to 5.0 mol and preferably 0.5 to 2.0 mol, with respect to 1 mol of the compound (I-1).

The reaction between the compound (I-1) and the compound (I-2) may be performed in the presence of a solvent. Examples of the solvent include acetonitrile, benzene, toluene, acetone, ethyl acetate, chloroform, dichloroethane, monochlorobenzene, methanol, ethanol, isopropanol, tert-butanol, 2-butanone, tetrahydrofuran, diethyl ether, dimethyl sulfoxide, N,N-dimethylacetamide, N,N-dimethylformamide, and water. Acetonitrile, tetrahydrofuran, chloroform, dichloromethane, and diethyl ether are preferable, acetonitrile, tetrahydrofuran, and chloroform are more preferable, and acetonitrile is still more preferable.

The solvent is preferably a dehydrated solvent.

The reaction time between the compound (I-1) and the compound (I-2) is usually 0.1 to 10 hours, and preferably 0.2 to 3 hours.

The reaction temperature between the compound (I-1) and the compound (I-2) is usually −50 to 150° C., and preferably −20 to 100° C.

The use amount of the compound (I-2) is usually 0.1 to 10 mol, and preferably 0.5 to 5 mol, with respect to 1 mol of the compound (I-1).

Examples of the compound (I-1) include the compounds described below.

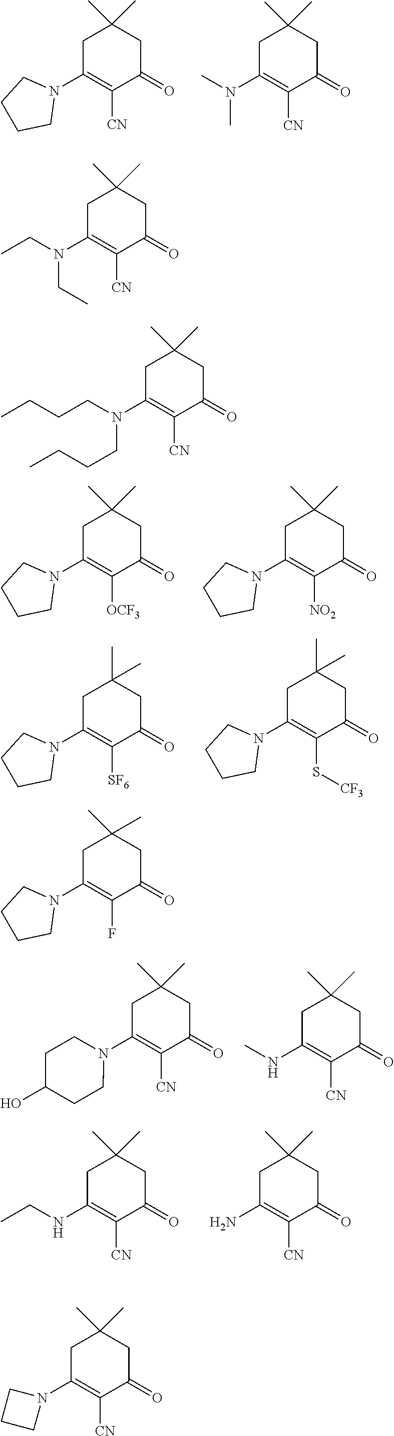

As the compound (I-2), a commercially available product may be used, and examples thereof include the compounds described below.

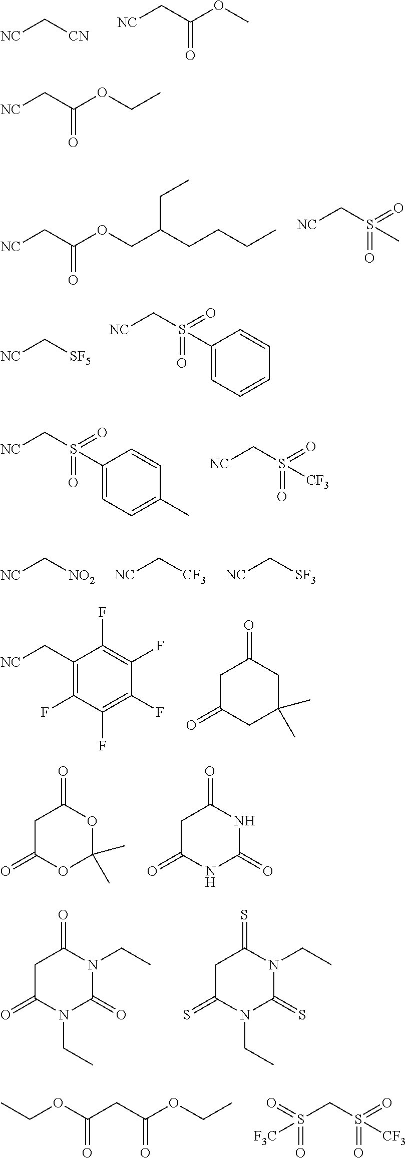

The compound (I-1) can be obtained, for example, by reacting a compound represented by Formula (I-3) (hereinafter, may be referred to as a compound (I-3)) with a compound represented by Formula (I-4) (hereinafter, may be referred to as a compound (I-4)).

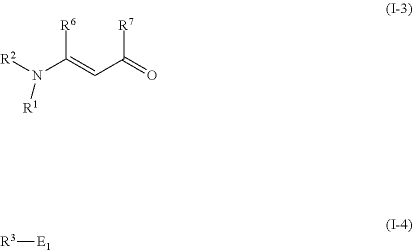

[in the formula, R¹, R², R³, R⁶, and R⁷ have the same meaning as described above. E₁ represents a leaving group].

Examples of the leaving group represented by E₁ include a halogen atom, a p-toluenesulfonyl group, and a trifluoromethylsulfonyl group.

The reaction between the compound (I-3) and the compound (I-4) is performed by mixing the compound (I-3) and the compound (I-4).

The use amount of the compound (I-4) is usually 0.1 to 5 mol, and preferably 0.5 to 2 mol, with respect to 1 mol of the compound (I-3).

The reaction between the compound (I-3) and the compound (I-4) may be performed in the presence of a solvent. Examples of the solvent include acetonitrile, benzene, toluene, acetone, ethyl acetate, chloroform, dichloroethane, monochlorobenzene, methanol, ethanol, isopropanol, tert-butanol, 2-butanone, tetrahydrofuran, diethyl ether, dimethyl sulfoxide, N,N-dimethylacetamide, N,N-dimethylformamide, and water. Acetonitrile, tetrahydrofuran, chloroform, dichloromethane, and diethyl ether are preferable, acetonitrile, tetrahydrofuran, and chloroform are more preferable, and methanol, ethanol, isopropanol, and acetonitrile are still more preferable.

The reaction time between the compound (I-3) and the compound (I-4) is usually 0.1 to 10 hours.

The reaction temperature between the compound (I-3) and the compound (I-4) is usually −50 to 150° C.

Examples of the compound (I-3) include the compounds described below.

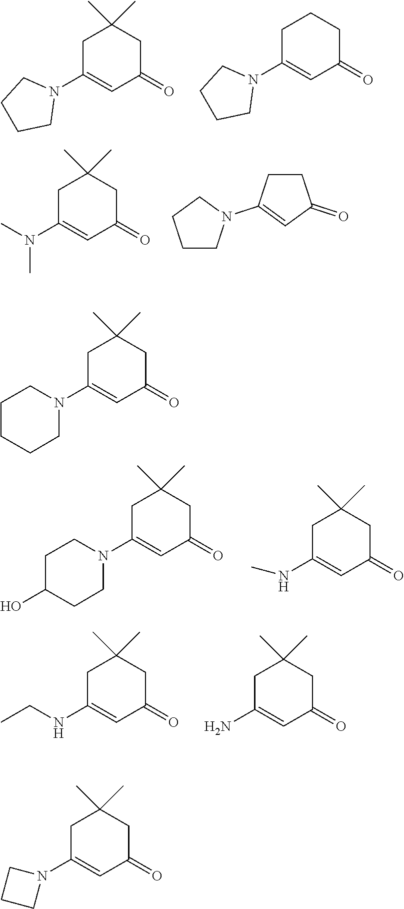

As the compound (I-4), a commercially available product may be used. Examples thereof include chlorocyanine, bromocyanine, p-toluenesulfonylcyanide, trifluoromethanesulfonylcyanide, 1-chloromethyl-4 fluoro-1,4-diazoniabicyclo [2.2.2] octane bis(tetrafluoroborate (also referred to as selected fluoro (registered trademark of Air Products and Chemicals)), benzoyl (phenyliodonio) (trifluoromethanesulfonyl) methanide, 2,8-difluoro-5-(trifluoromethyl)-5H-dibenzo [b,d] thiophene-5-ium trifluoromethanesulfonate, N-bromosuccinimide, N-chlorosuccinimide, and N-iodosuccinimide.

The compound (I-3) can be obtained, for example, by reacting a compound represented by Formula (I-5) (hereinafter, may be referred to as a compound (I-5)) with a compound represented by Formula (I-6) (hereinafter, may be referred to as a compound (I-6)).

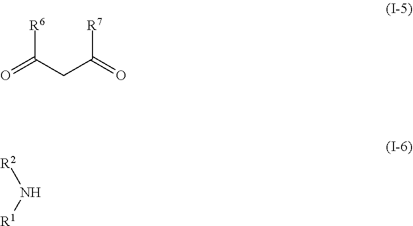

[in the formula, R¹, R², R⁶, and R⁷ have the same meaning as described above].

In Formula (I-5), and R⁷ may be linked to each other to form a ring, and the ring in this case may or may not contain a double bond as a constituent element of the ring.

The reaction between the compound (I-5) and the compound (I-6) is performed by mixing the compound (I-5) and the compound (I-6).

The use amount of the compound (I-6) is usually 0.1 to 5 mol, and preferably 0.5 to 2 mol, with respect to 1 mol of the compound (I-5).

The reaction between the compound (I-5) and the compound (I-6) may be performed in the presence of a solvent. Examples of thereof include acetonitrile, benzene, toluene, acetone, ethyl acetate, chloroform, dichloroethane, monochlorobenzene, methanol, ethanol, isopropanol, tert-butanol, 2-butanone, tetrahydrofuran, diethyl ether, dimethyl sulfoxide, N,N-dimethylacetamide, N,N-dimethylformamide, and water. Benzene, toluene, ethanol, and acetonitrile are preferable.

The reaction time between the compound (I-5) and the compound (I-6) is usually 0.1 to 10 hours.

The reaction temperature between the compound (I-5) and the compound (I-6) is usually −50 to 150° C.

Examples of the compound (I-5) include the compounds described below.

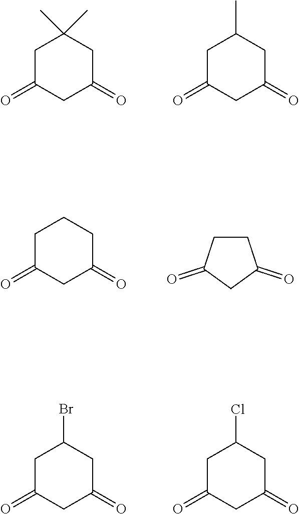

Examples of the compound (I-6) include primary amines such as methylamine, ethylamine, ethanolamine, and 4-hydroxybutylamine; secondary amines such as dimethylamine, diethylamine, dibutylamine, pyrrolidine, piperidine, 3-hydroxypyrrolidine, and 4-hydroxypiperidine.

The compound (I-1) can also be obtained by reacting compound (hereinafter, may be referred to as a compound (I-5-1)) represented by Formula (I-5-1) with a compound (I-6).

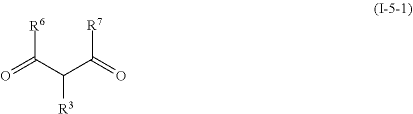

[in Formula (I-5-1), R³, and R⁷ have the same meanings as described above].

In Formula (I-5-1), R⁶ and R⁷ may be linked to each other to form a ring, and the ring in this case may or may not contain a double bond as a constituent element of the ring.

The reaction between the compound (I-5-1) and the compound (I-6) is performed by mixing the compound (I-5-1) and the compound (I-6).

The use amount of the compound (I-6) is usually 0.1 to 5 mol, and preferably 0.5 to 2 mol, with respect to 1 mol of the compound (I-5-1).

The reaction between the compound (I-5-1) and the compound (I-6) may be performed in the presence of a solvent. Examples of thereof include acetonitrile, benzene, toluene, acetone, ethyl acetate, chloroform, dichloroethane, monochlorobenzene, methanol, ethanol, isopropanol, tert-butanol, 2-butanone, tetrahydrofuran, diethyl ether, dimethyl sulfoxide, N,N-dimethylacetamide, N,N-dimethylformamide, and water. Benzene, toluene, ethanol, and acetonitrile are preferable.

The reaction time between the compound (I-5-1) and the compound (I-6) is usually 0.1 to 10 hours.

The reaction temperature between the compound (I-5-1) and the compound (I-6) is usually −50 to 150° C.

Examples of the compound represented by Formula (I-5-1) include the compounds described below.

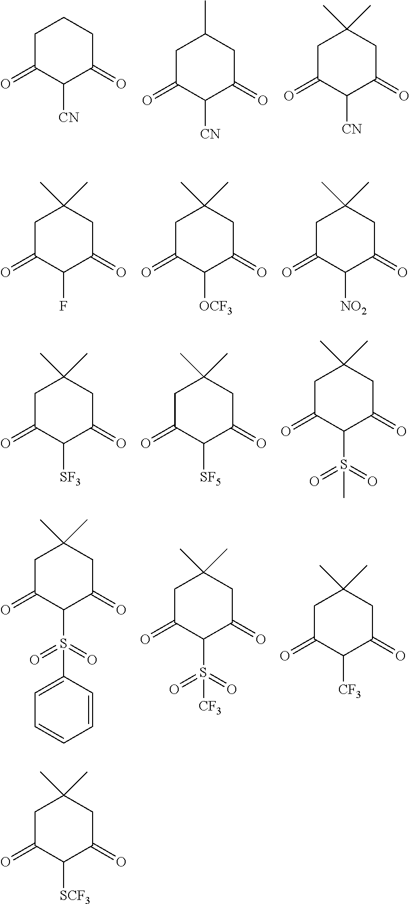

The compound (I) can also be obtained by reacting compound (hereinafter, may be referred to as a compound (I-7)) represented by Formula (I-7) with a compound (I-6).

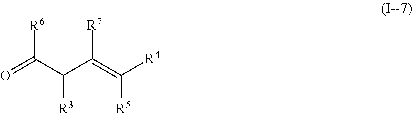

[in Formula (I-7), R³ to R⁷ has the same meaning as described above].

In Formula (I-7), and R⁷ may be linked to each other to form a ring, and the ring in this case may or may not contain a double bond as a constituent element of the ring.

The reaction between the compound (I-7) and the compound (I-6) is usually performed by mixing the compound (I-7) and the compound (I-6), and it is preferable to add the compound (I-7) to the compound (I-6).

The reaction between the compound (I-7) and the compound (I-6) is preferably performed by mixing the compound (I-7) and the compound (I-6) in the presence of a base and a methylating agent,

it is more preferable to mix the compound (I-7), the compound (I-6), a base, and a methylating agent, and

it is more preferable to mix the compound (I-7) with a mixture of the compound (I-6), a methylating agent, and a base.

Examples of the base include the same bases as those used for the reaction between the compound (I-1) and the compound (I-2).

The use amount of the base is usually 0.1 to 5.0 mol and preferably 0.5 to 2.0 mol, with respect to 1 mol of the compound (I-7).

Examples of the methylating agent include the same methylating agents as those used for the reaction between the compound (I-1) and the compound (I-2).

The use amount of the methylating agent is usually 0.1 to 5.0 mol and preferably 0.5 to 2.0 mol, with respect to 1 mol of the compound (I-7).

The use amount of the compound (I-6) is usually 0.1 to 10 mol, and preferably 0.5 to 5 mol, with respect to 1 mol of the compound (I-7).

The reaction between the compound (I-7) and the compound (I-6) may be performed in the presence of a solvent. Examples of the solvent include the same solvents as those used for the reaction between the compound (I-1) and the compound (I-2). Methanol, ethanol, isopropanol, toluene, and acetonitrile are preferable.

The reaction time between the compound (I-7) and the compound (I-6) is usually 0.1 to 10 hours.

The reaction temperature between the compound (I-7) and the compound (I-6) is usually −50 to 150° C.

Examples of the compound (I-7) include the compounds described below.

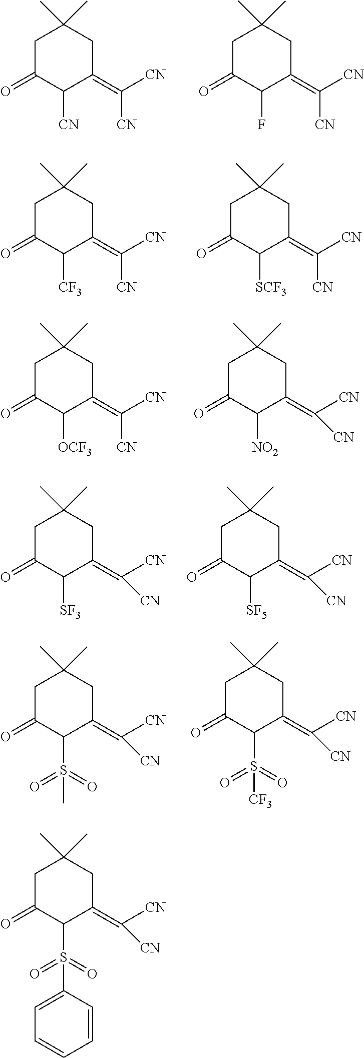

The compound (I-7) can also be obtained by reacting compound represented by Formula (I-8) with a compound (I-4).

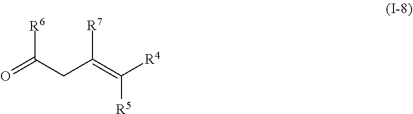

[in Formula (I-8), R⁴ to R⁷ has the same meaning as described above].

In Formula (I-8), R⁶ and R⁷ may be linked to each other to form a ring, and the ring in this case may or may not contain a double bond as a constituent element of the ring.

The reaction between the compound (I-8) and the compound (I-4) can be performed by mixing the compound (I-8) and the compound (I-4).

The reaction between the compound (I-8) and the compound (I-4) is preferably performed in the presence of a base. Examples of the base include the same bases as those used for the reaction between the compound (I-1) and the compound (I-2). A metal hydroxide, a metal alkoxide, an amine compound, or a metal amide compound is preferable.

The use amount of the base is usually 0.1 to 10 mol and preferably 0.5 to 2.0 mol, with respect to 1 mol of the compound (I-8).

The reaction between the compound (I-8) and the compound (I-4) may be performed in the presence of a solvent. Examples of the solvent include the same solvents as those used for the reaction between the compound (I-1) and the compound (I-2). Toluene, acetonitrile, methanol, ethanol, and isopropanol are preferable.

The reaction time between the compound (I-8) and the compound (I-4) is usually 0.1 to 10 hours.

The reaction temperature between the compound (I-8) and the compound (I-4) is usually −50 to 150° C.

Examples of the compound (I-8) include the compounds described below.

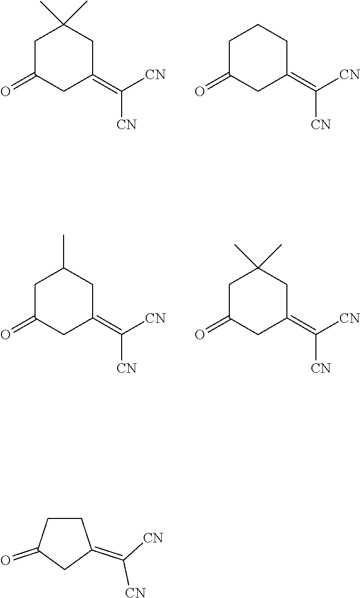

The compound (I-8) can also be obtained by reacting the compound (I-5) with the compound (I-2). The reaction between the compound (I-5) and the compound (I-2) can be performed by mixing the compound (I-5) and the compound (I-2).

The reaction between the compound (I-5) and the compound (I-2) is preferably performed in the presence of a base. Examples of the base include the same bases as those used for the reaction between the compound (I-1) and the compound (I-2). The use amount of the base is usually 0.1 to 5 mol and preferably 0.5 to 2 mol, with respect to 1 mol of the compound (I-5).

The reaction between the compound (I-5) and the compound (I-2) may be performed in the presence of a solvent. Examples of the solvent include the same solvents as those used for the reaction between the compound (I-1) and the compound (I-2). Methanol, ethanol, isopropanol, toluene, and acetonitrile are preferable.

The reaction time between the compound (I-5) and the compound (I-2) is usually 0.1 to 10 hours.

The reaction temperature between the compound (I-5) and the compound (I-2) is usually −50 to 150° C.

The use amount of the compound (I-2) is usually 0.1 to 10 mol, and preferably 0.5 to 2 mol, with respect to 1 mol of the compound (I-5).

The compound (I) can also be obtained by reacting compound (hereinafter, may be referred to as a compound (I-9)) represented by Formula (1-9) with a compound (I-4).

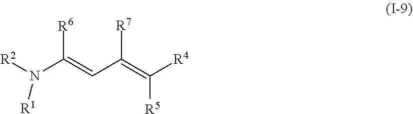

[in Formula (I-9), R¹, R², and R⁴ to R⁷ have the same meaning as described above].

The reaction between the compound (I-9) and the compound (I-4) is performed by mixing the compound (I-9) and the compound (I-4).

The use amount of the compound (I-4) is usually 0.1 to 5 mol, and preferably 0.5 to 2 mol, with respect to 1 mol of the compound (I-9).

The reaction between the compound (I-9) and the compound (I-4) may be performed in the presence of a solvent. Examples of the solvent include acetonitrile, benzene, toluene, acetone, ethyl acetate, chloroform, dichloroethane, monochlorobenzene, methanol, ethanol, isopropanol, tert-butanol, 2-butanone, tetrahydrofuran, diethyl ether, dimethyl sulfoxide, N,N-dimethylacetamide, N,N-dimethylformamide, and water. Acetonitrile, tetrahydrofuran, chloroform, dichloromethane, and diethyl ether are preferable, acetonitrile, tetrahydrofuran, and chloroform are more preferable, and methanol, ethanol, isopropanol, and acetonitrile are still more preferable.

The reaction time between the compound (I-9) and the compound (I-4) is usually 0.1 to 10 hours.

The reaction temperature between the compound (I-9) and the compound (I-4) is usually −50 to 150° C.

Examples of the compound represented by Formula (I-9) include the compounds described below.

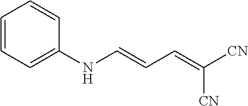 [SMILES: N#CC(C#N)=C/C=C/Nc1ccccc1]

The compound represented by Formula (I-9) can also be obtained by reacting compound represented by Formula (I-10) with a compound (I-2).

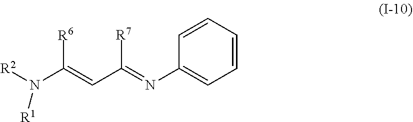

[in Formula (I-10), R¹, R², R⁶, and have the same meaning as described above].

The reaction between the compound (I-10) and the compound (I-2) is performed by mixing the compound (I-10) and the compound (I-2).

The use amount of the compound (I-2) is usually 0.1 to 5 mol, and preferably 0.5 to 2 mol, with respect to 1 mol of the compound (I-10).

The reaction between the compound (I-10) and the compound (I-2) may be performed in the presence of a solvent. Examples of thereof include acetonitrile, benzene, toluene, acetone, ethyl acetate, chloroform, dichloroethane, monochlorobenzene, methanol, ethanol, isopropanol, tert-butanol, 2-butanone, tetrahydrofuran, diethyl ether, dimethyl sulfoxide, N,N-dimethylacetamide, N,N-dimethylformamide, and water. Benzene, toluene, ethanol, and acetonitrile are preferable.

The reaction time between the compound (I-10) and the compound (I-2) is usually 0.1 to 10 hours.

The reaction temperature between the compound (I-10) and the compound (I-2) is usually −50 to 150° C.

Examples of the compound represented by Formula (I-10) include the compounds described below, and the compound can be obtained as commercially available malonaldehyde dianilide hydrochloride.

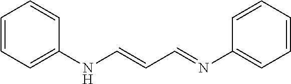 [SMILES: C(=C\Nc1ccccc1)/C=N/c1ccccc1]

<Composition Containing Compound (I)>

The present invention also includes a composition containing compound (I).

The composition containing the compound (I) of the present invention is preferably a resin composition containing the compound (I) and a resin.

The composition the composition can be particularly suitably used for applications that may be exposed to sunlight or light including ultraviolet rays. Specific examples thereof include glass substitutes and surface coating materials thereof; coating materials for window glass, lighting glass, and light source protective glass for housing, facility, transport equipment and the like; window films for housing, facility, transport equipment and the like; interior/exterior materials and interior/exterior paints for housing, facility, transport equipment and the like, and coating films formed by the paint; alkyd resin lacquer paints and coating films formed by the paint; acrylic lacquer paints and coating films formed by the paint; light source members that emit ultraviolet rays, such as a fluorescent lamp and a mercury lamp; materials for blocking electromagnetic waves generated from precision machinery, electronic and electrical equipment members, and various displays; containers or packaging materials for foods, chemicals, chemicals, and the like; bottles, boxes, blisters, cups, special packaging, compact disc coats, agricultural and industrial sheets, or film materials; anti-fading agents for a printed matter, a dyed matter, a dyeing pigment, and the like; protective films for polymer supports (for example, for plastic parts such as machine and automotive parts); printed matter overcoat; inkjet medium coating; laminated mattes; optical light films; safety glass/windshield intermediate layers; electrochromic/photochromic applications; overlaminated films; solar heat control films; cosmetics such as sunscreen, shampoo, conditioner, and hair styling products; textile products and textiles for clothing such as sportswear, stockings and hats; household interiors such as curtains, rugs, and wallpaper; medical instruments such as plastic lenses, contact lenses, and artificial eyes; optical supplies such as optical filters, backlight display films, prisms, mirrors, and photographic materials; stationery such as mold films, transfer stickers, graffiti prevention films, tapes, and inks; and marking boards, signing device, and the like, and surface coating materials thereof.

A shape of a polymer molded article formed from the resin composition may be any shape such as a flat film shape, a powder shape, a spherical particle shape, a crushed particle shape, a massive continuous body, a fiber shape, a tubular shape, a hollow fiber shape, a granular shape, a plate shape, and a porous shape.

Examples of the resin used in the resin composition include a thermoplastic resin and a thermosetting resin conventionally used in the production of known various molded bodies, sheets, films and the like.

Examples of the thermoplastic resin include olefin resins such as a polyethylene resin, a polypropylene resin, and a polycycloolefin resin, polyester resins such as a poly (meth)acrylic acid ester resin, a polystyrene resin, a styrene-acrylonitrile resin, an acrylonitrile-butadiene-styrene resin, a polyvinyl chloride resin, a polyvinylidene chloride resin, a polyvinyl acetate resin, a polyvinyl butyral resin, an ethylene-vinyl acetate copolymer, an ethylene-vinyl alcohol resin, a polyethylene terephthalate resin, a polybutylene terephthalate resin, and a liquid crystal polyester resin, a polyacetal resin, a polyamide resin, a polycarbonate resin, a polyurethane resin, and a polyphenylene sulfide resin. These resins may be used as one or more polymer blends or polymer alloys.

Examples of the thermosetting resin include an epoxy resin, a melamine resin, an unsaturated polyester resin, a phenol resin, a urea resin, an alkyd resin, and a thermosetting polyimide resin.

When the resin composition is used as an ultraviolet absorbing filter or an ultraviolet absorbing film, the resin is preferably a transparent resin.

The resin composition can be obtained by mixing a compound (I) and a resin. The compound (I) only needs to be contained in an amount necessary for imparting desired performance, and can be contained, for example, in an amount of 0.01 to 20 parts by mass or the like with respect to 100 parts by mass of the resin.

The composition of the present invention may contain other additives such as a solvent, a crosslinking catalyst, a tackifier, a plasticizer, a softener, a dye, a pigment, and an inorganic filler as necessary.

The composition and the resin composition may be a spectacle lens composition. A spectacle lens can be formed by molding or the like using the spectacle lens composition. The method for molding the spectacle lens composition may be injection molding or cast polymerization molding. The cast polymerization molding is a method in which a spectacle lens composition mainly composed of a monomer or oligomer resin is injected into a lens mold, and the spectacle lens composition is cured by heat or light to be molded into a lens.

The spectacle lens composition may have a composition suitable for the molding method. For example, when the spectacle lens is formed by injection molding, a resin composition for a spectacle lens containing the resin and the compound (I) may be used. In addition, when the spectacle lens is formed by cast polymerization molding, a spectacle lens composition containing a curable monomer that cures by heat or light and the compound (I) may be used.

Examples of the resin contained in the spectacle lens composition include the resins described above, and a transparent resin is preferable. The resin contained in the spectacle lens composition is preferably used as a polymer blend or a polymer alloy of one or more of a poly (meth)acrylic acid ester resin, a polycarbonate resin, a polyamide resin, a polyurethane resin, and a polythiourethane resin. In addition, not only the polymer but also a monomer component may be contained.

The spectacle lens composition may be a composition containing the curable monomer and the compound (I). Two or more curable monomers may be contained. Specifically, it may be a mixture of a polyol compound and an isocyanate compound or a mixture of a thiol compound and an isocyanate compound, and is preferably a mixture of a thiol compound and an isocyanate, and more preferably a mixture of a polyfunctional thiol compound and a polyfunctional isocyanate compound.

The thiol compound is not particularly limited as long as it is a compound having at least one thiol group in the molecule. It may be chain or cyclic. In addition, a sulfide bond, a polysulfide bond, and other functional groups may be present in the molecule. Specific examples of the thiol compound include thiol group-containing organic compounds having one or more thiol groups in one molecule described in JP-A-2004-315556, such as an aliphatic polythiol compound, an aromatic polythiol compound, a thiol group-containing cyclic compound, and a thiol group-containing sulfide compound. Among them, from the viewpoint of improving the refractive index and the glass transition temperature of the optical material, a polyfunctional thiol compound having two or more thiol groups is preferable, an aliphatic polythiol compound having two or more thiol groups and a sulfide compound containing two or more thiol groups are more preferable, and bis(mercaptomethyl) sulfide, 1,2-bis[(2-mercaptoethyl) thio]-3 mercaptopropane, pentaerythritol tetrakisthiopropionate, and 4,8-dimercaptomethyl-1,11 mercapto-3,6,9-trithiaundecane are still more preferable. The thiol compounds may be used alone or in combination of two or more thereof.

The isocyanate compound is preferably a polyfunctional isocyanate compound having at least two isocyanato groups (—NCO) in the molecule, and examples thereof include an aliphatic isocyanate compound (for example, hexamethylene diisocyanate), an alicyclic isocyanate compound (for example, isophorone diisocyanate, hydrogenated diphenylmethane diisocyanate, and hydrogenated xylylene diisocyanate), and an aromatic isocyanate compound (for example, tolylene diisocyanate, xylylene diisocyanate, diphenylmethane diisocyanate, naphthalene diisocyanate, and triphenylmethane triisocyanate). In addition, the isocyanate compound may be an adduct of the isocyanate compound with a polyhydric alcohol compound [adducts with, for example, glycerol, and trimethylolpropane], a derivative of an isocyanurate compound, a biuret type compound, and a urethane prepolymer type isocyanate compound obtained by addition reaction with polyether polyol, polyester polyol, acrylic polyol, polybutadiene polyol, polyisoprene polyol, or the like.

When the spectacle lens composition contains a curable monomer, a curing catalyst may be contained in order to improve curability. Examples of the curing catalyst include a tin compound such as dibutyltin chloride, amines disclosed in JP-A-2004-315556, phosphines, quaternary ammonium salts, quaternary phosphonium salts, tertiary sulfonium salts, secondary iodonium salts, mineral acids, Lewis acids, organic acids, silicic acids, tetrafluoroboric acids, peroxides, an azo based compound, a condensate of aldehyde and an ammonia compound, guanidines, thioureas, thiazoles, sulfenamides, thiurams, dithiocarbamates, xanthogenates, and acidic phosphoric esters. These curing catalysts may be used alone or in combination of two or more thereof.

When the spectacle lens composition is a resin composition, the content of the compound (I) in the spectacle lens composition can be, for example, 0.01 to 20 parts by mass based on 100 parts by mass of the resin. When the spectacle lens composition is a curable composition, for example, the content of the compound (I) can be 0.00001 to 20 parts by mass based on 100 parts by mass of the curable component. The content of the compound (I) is preferably 0.0001 to 15 parts by mass, more preferably 0.001 to 10 parts by mass, still more preferably 0.01 to 5 parts by mass, and particularly preferably 0.1 to 3 parts by mass, based on 100 parts by mass of the resin or the curable component.

The addition amount of the curing catalyst is preferably 0.0001 to 10.0% by mass, and more preferably 0.001 to 5.0% by mass based on 100% by mass of the spectacle lens composition.

The spectacle lens composition may contain the above-described additives.

### EXAMPLES

Hereinafter, the present invention will be described more specifically with reference to examples and comparative examples, but the present invention is not limited by these examples. In the examples, % and part representing the content or use amount are on a mass basis unless otherwise specified.

### (Example 1) Synthesis of Compound Represented by Formula (UVA-6)

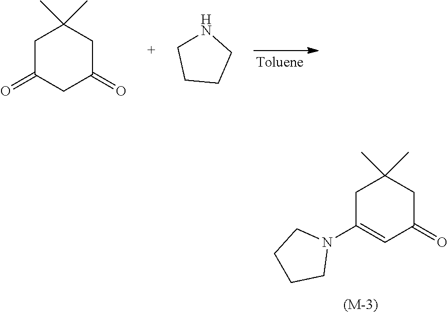

The inside of a 500 mL-four-necked flask equipped with a Dimroth cooling tube and a thermometer was made into a nitrogen atmosphere, and 20 parts of dimedone, 11.2 parts of pyrrolidine, and 200 parts of toluene were added thereto, and the mixture was stirred under reflux for 5 hours. The solvent was distilled off from the obtained mixture and purification was performed to obtain 27.4 parts of a compound represented by Formula (M-3).

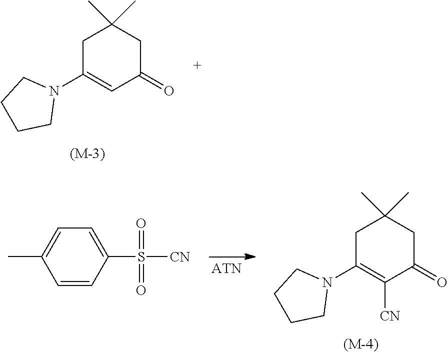

Under a nitrogen atmosphere, 1.0 part of the obtained compound represented by Formula (M-3), 2.8 parts of p-toluenesulfonyl cyanide, and 10 parts of acetonitrile were mixed. The obtained mixture was allowed to stir at 0 to 5° C. for 5 hours. The solvent was distilled off from the obtained mixture and purified to obtain 0.6 parts of a compound represented by Formula (M-4).

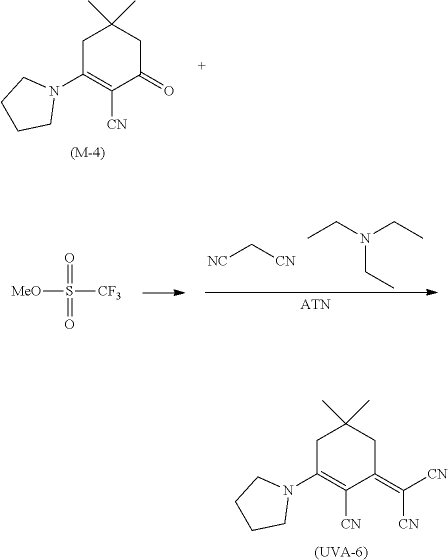

Under a nitrogen atmosphere, 4.8 parts of a compound represented by Formula (M-4), 4.6 parts of methyl triflate, and 24 parts of acetonitrile were mixed, and the mixture was stirred at 20 to 30° C. for 3 hours. 1.9 parts of malononitrile, 3 parts of triethylamine, and 24 parts of acetonitrile were added to the obtained mixture, and the mixture was stirred at 20 to 30° C. for 3 hours. The solvent was distilled off from the obtained mixture and purified to obtain 2.9 parts of a compound represented by Formula (UVA-6).

LC-MS measurement and ¹H-NMR analysis were performed, and it was confirmed that a compound represented by Formula (UVA-6) was produced.

¹H-NMR (CDCl₃) δ: 0.99 (s, 6H), 1.90-1.96 (m, 4H), 2.48-2.51 (m, 4H), 3.70-3.88 (dt, 4H)

LC-MS; [M+H]⁺=284.5

<Measurement of Maximum Absorption Wavelength and Gram Absorption Coefficient ε>

A 2-butanone solution (0.006 g/L) of the obtained compound represented by Formula (UVA-6) was charged into a 1 cm quartz cell, the quartz cell was set in a spectrophotometer UV-2450 (manufactured by Shimadzu Corporation), and absorbance in a wavelength range of 300 to 800 nm was measured every 1 nm step by a double beam method. From the obtained absorbance value, the concentration of the compound represented by Formula (UVA-6) in the solution, and an optical path length of the quartz cell, a gram absorption coefficient for each wavelength was calculated.

ε(λ)=A(λ)/CL

[in the equation, ε(λ) represents a gram absorption coefficient (L/(g·cm)) of the compound represented by Formula (UVA-6) at a wavelength of λ nm, A(λ) represents absorbance at a wavelength of λ nm, C represents concentration (g/L), and L represents an optical path length (cm) of the quartz cell].

The maximum absorption wavelength of the obtained compound represented by Formula (UVA-6) was 380 nm. The obtained compound represented by Formula (UVA-6) had ε(λ max) of 1.75 L/(g·cm), ε(λ max+30 nm) of 0.032 L/(g·cm), and ε(λ max)/ε(λ max+30 nm) of 54.53.

### (Example 2) Synthesis of Compound Represented by Formula (UVA-7)

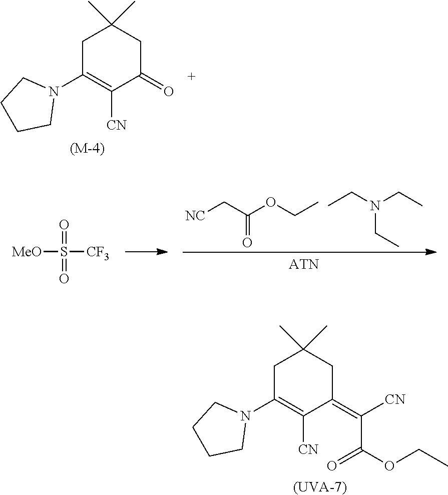

Under a nitrogen atmosphere, 1 part of a compound represented by Formula (M-4), 0.6 parts of methyl triflate, and 10 parts of acetonitrile were mixed, and the mixture was stirred at 20 to 30° C. for 3 hours. 5.2 parts of ethyl cyanoacetate, 4.6 parts of triethylamine, and 10 parts of acetonitrile were added to the obtained mixture, and the mixture was stirred at 20 to 30° C. for 3 hours. The solvent was distilled off from the obtained mixture and purified to obtain 0.5 parts of a compound represented by Formula (UVA-7).

LC-MS measurement and ¹H-NMR analysis were performed, and it was confirmed that a compound represented by Formula (UVA-7) was produced.

¹H-NMR (deuterated DMSO) δ: 0.960-0.994 (d, 6H), 1.20-1.26 (m, 3H), 1.93 (m, 4H), 2.53-2.91 (m, 4H), 3.77-3.81 (m, 4H), 4.10-4.19 (m, 2H)

LC-MS; [M+H]⁺=314.5(+H)

In addition, the maximum absorption wavelength and the gram absorption coefficient were measured in the same manner as described above. The maximum absorption wavelength of the obtained compound represented by Formula (UVA-7) was 382.7 nm. The obtained compound represented by Formula (UVA-7) had ε(λ max) of 1.08 L/(g·cm), ε(λ max+30 nm) of 0.153 L/(g·cm), and ε(λ max)/ε(λ max+30 nm) of 7.04.

### (Example 3) Synthesis of Compound Represented by Formula (UVA-8)

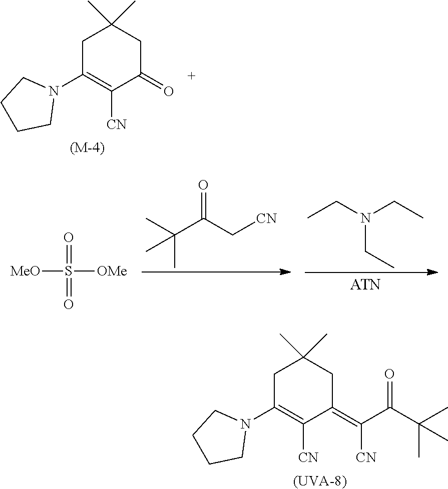

Under a nitrogen atmosphere, 0.5 parts of the compound represented by Formula (M-4), 0.5 parts of dimethyl sulfuric acid, and 5 parts of acetonitrile were mixed, and the mixture was stirred and reacted at 20 to 30° C. for 3 hours. Further, 0.4 parts of pivaloylacetonitrile, 0.5 parts of triethylamine, and 5.0 parts of acetonitrile were added, and the mixture was stirred and reacted at 20 to 30° C. for 3 hours. After completion of the reaction, the solvent was distilled off and purified to obtain 0.07 parts of a compound represented by Formula (UVA-8).

LC-MS measurement and ¹H-NMR analysis were performed, and it was confirmed that a compound represented by Formula (UVA-8) was produced.

¹H-NMR (deuterated DMSO) δ: 0.92 (s, 6H), 1.26 (s, 9H), 1.90 (s, 4H), 2.55 (m, 4H), 3.64-3.71 (m, 4H)

LC-MS; [M+H]⁺=326.5

In addition, the maximum absorption wavelength and the gram absorption coefficient were measured in the same manner as described above. The maximum absorption wavelength of the obtained compound represented by Formula (UVA-8) was 377.4 nm. The obtained compound represented by Formula (UVA-8) had ε(max) of 0.66 L/(g·cm), ε(λ max+30 nm) of 0.395 L/(g·cm), and ε(λ max)/ε(λ max+30 nm) of 1.68.

### (Example 4) Synthesis of Compound Represented by Formula (UVA-9)

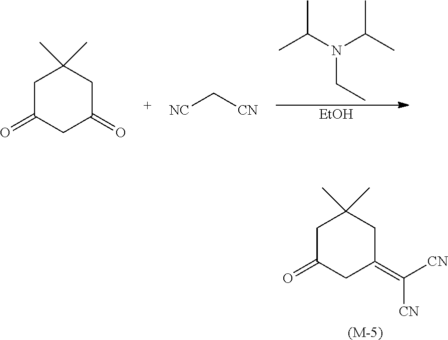

The inside of a 300 rat-four-necked flask equipped with a Dimroth cooling tube and a thermometer was made into a nitrogen atmosphere, and 70.0 parts of dimedone, 10.4 parts of malononitrile, 40.6 parts of diisopropylethylamine, and 100.0 parts of ethanol were charged thereto, and the mixture was heated and stirred under reflux for 3 hours. After completion of the reaction, the solvent was distilled off and purified to obtain 15.1 parts of a compound represented by Formula (M-5).

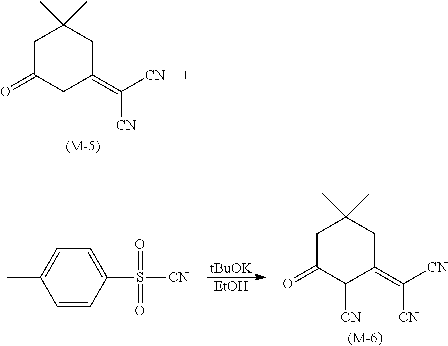

Under a nitrogen atmosphere, 5 parts of the compound represented by Formula (M-5), 5.8 parts of p-toluenesulfonyl cyanide, 3 parts of potassium tert-butoxide, and 50 parts of ethanol were mixed. The obtained mixture was allowed to stir at 0 to 5° C. for 3 hours. The solvent was distilled off from the obtained mixture and purified to obtain 3.3 parts of a compound represented by Formula (M-6).

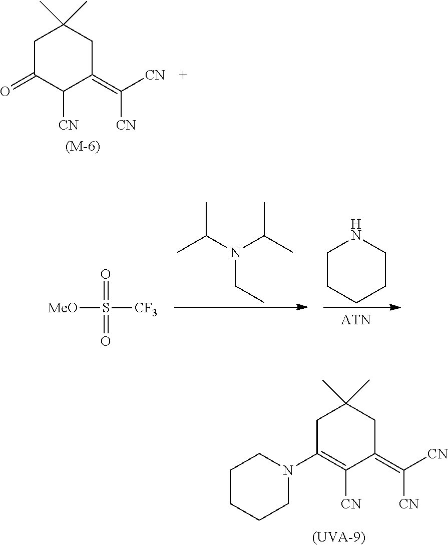

Under a nitrogen atmosphere, 1 part of a compound represented by Formula (M-6), 1 part of methyl triflate, 0.8 parts of diisopropylethylamine and 20 parts of acetonitrile were mixed, and the mixture was stirred at 20 to 30° C. for 3 hours. 1.4 parts of piperidine and 20 parts of acetonitrile were added to the obtained mixture, and the mixture was stirred at 20 to 30° C. for 3 hours. The solvent was distilled off from the obtained mixture and purified to obtain 0.5 parts of a compound represented by Formula (UVA-9).

LC-MS measurement and ¹H-NMR analysis were performed, and it was confirmed that a compound represented by Formula (UVA-9) was produced.

¹H-NMR (deuterated DMSO) δ: 0.99 (s, 6H), 1.60 (m, 6H), 2.71 (s, 2H), 3.80 (m, 4H)

LC-MS; [M+H]⁺=281.5

In addition, the maximum absorption wavelength and the gram absorption coefficient were measured in the same manner as described above. The maximum absorption wavelength of the obtained compound represented by Formula (UVA-9) was 385.6 nm. The obtained compound represented by Formula (UVA-9) had ε(λ max) of 1.65 L/(g·cm), ε(λ max+30 nm) of 0.088 L/(g·cm), and ε(λ max)/ε(λ max+30 nm) of 18.8.

### (Synthesis Example 1) Synthesis of Compound Represented by Formula (UVA-A1)

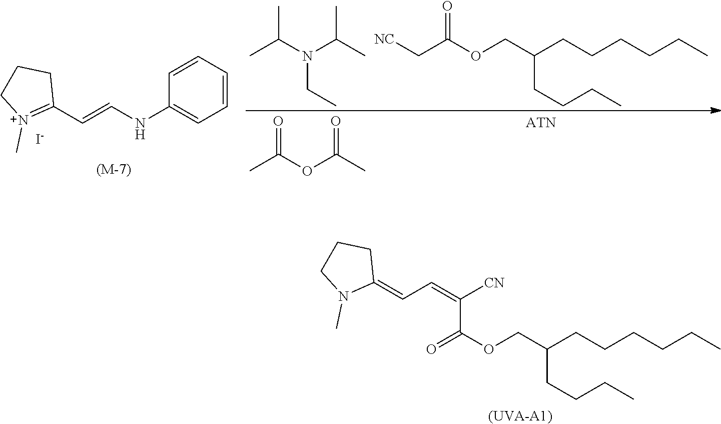

The inside of a 200 mL-four-necked flask equipped with a Dimroth cooling tube and a thermometer was made into a nitrogen atmosphere, and 10 parts of a compound represented by Formula (M-7) synthesized with reference to JP-A-2014-194508, 3.6 parts of acetic anhydride, 6.9 parts of (2-butyloctyl) cyanoacetate, and 60 parts of acetonitrile were charged, and the obtained mixture was stirred at 20 to 30° C. 4.5 parts of diisopropylethylamine was added dropwise to the obtained mixture over 1 hour, and the mixture was stirred for 2 hours. The solvent was distilled off from the obtained mixture and purified to obtain 4.6 parts of a compound represented by Formula (UVA-A1).

### (Synthesis Example 2) Synthesis of Compound Represented by Formula (UVA-A2)

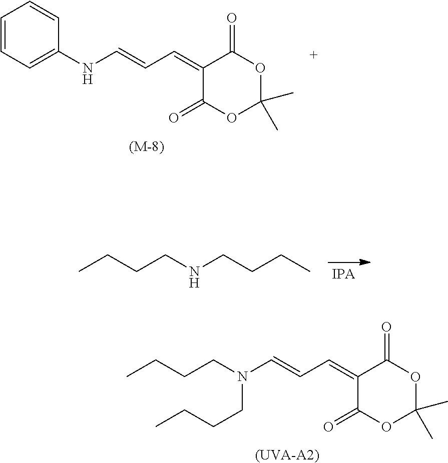

The inside of a 100 mL-four-necked flask equipped with a Dimroth cooling tube and a thermometer was made into a nitrogen atmosphere, and 6 parts of the compound represented by Formula (M-8), 14.2 parts of dibutylamine, and 31.3 parts of isopropanol were mixed, heated and refluxed, and then stirred for 3 hours. The solvent was distilled off from the obtained mixture and purified to obtain 4.6 parts of a compound represented by Formula (UVA-A2).

### (Synthesis Example 3) Synthesis of Compound Represented by Formula (UVA-A3)

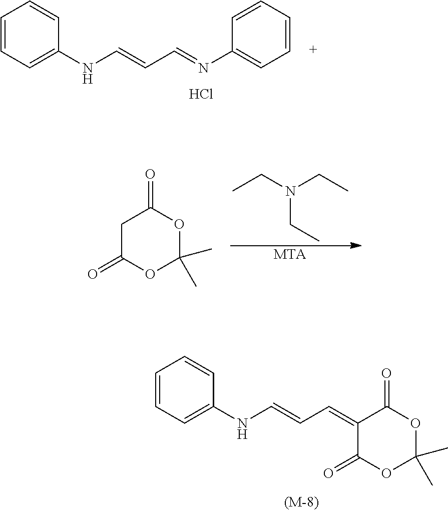

The inside of a 300 mL-four-necked flask equipped with a Dimroth cooling tube and a thermometer was made into a nitrogen atmosphere, and 30 parts of malonaldehyde dianilide hydrochloride, 18.4 parts of meldrum acid, 12.9 parts of triethylamine, and 90 parts of methanol were charged, and the mixture was stirred and reacted at 20 to 30° C. for 3 hours. After completion of the reaction, the solvent was distilled off and purified to obtain 24.4 parts of a compound represented by Formula (M-8).

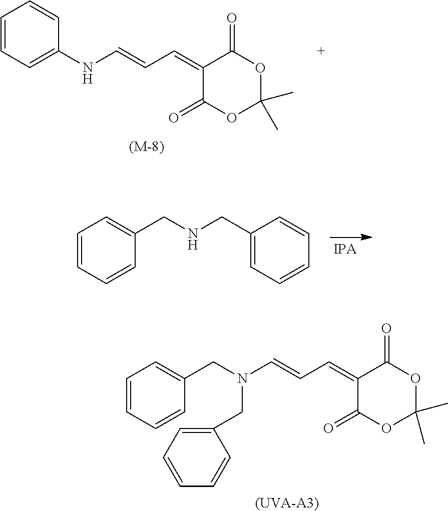

6 parts of the compound represented by Formula (M-8), 21.7 parts of dibenzylamine, and 31.3 parts of isopropanol were mixed, heated and refluxed, and the mixture was stirred and reacted for 3 hours. The solvent was distilled off from the obtained mixture and purified to obtain 3.5 parts of a compound represented by Formula (UVA-A3).

### (Synthesis Example 4) Synthesis of Compound Represented by Formula (UVA-A4)

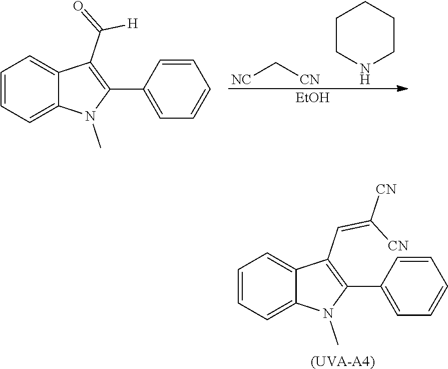

The inside of a 100 mL-four-necked flask equipped with a Dimroth cooling tube and a thermometer was made into a nitrogen atmosphere, and 5 parts of 2-phenyl-1 methylindole-3-carboxyaldehyde, 1.8 parts of piperidine, 1.5 parts of malononitrile, and 20 parts of ethanol were mixed, heated and refluxed, and the mixture was stirred for 18 hours. The obtained mixture was heated to 80° C. and kept at 80° C. for 18 hours. The solvent was distilled off from the obtained mixture and purified to obtain 4.9 parts of a compound represented by Formula (UVA-A4).

### (Example 5) Preparation of Light-Selective Absorption Composition (1)

The respective components were mixed in the following proportions to prepare a light-selective absorption composition (active energy ray-curable resin composition) (1).

Polyfunctional acrylate (“A-DPH-12E”: produced by Shin-Nakamura Chemical Co., Ltd.) 70 parts

Urethane acrylate (“UV-76503”: produced by Nippon Chemical Industrial Co., Ltd.) 30 parts

Polymerization initiator (“NCI-730”: produced by ADEKA Corporation) 3 parts

Compound Represented by Formula (UVA-6) Synthesized in Example 1 2 parts

Methyl ethyl ketone 34 parts

### (Example 6) Preparation of Light-Selective Absorption Composition (2)

A light-selective absorption composition (2) was prepared in the same manner as in Example 5 except that a compound represented by Formula (UVA-6) was changed to a compound represented by Formula (UVA-7).

### (Example 7) Preparation of Light-Selective Absorption Composition (3)

A light-selective absorption composition (3) was prepared in the same manner as in Example 5 except that a compound represented by Formula (UVA-6) was changed to a compound represented by Formula (UVA-8).

### (Example 8) Preparation of Light-Selective Absorption Composition (4)

A light-selective absorption composition (4) was prepared in the same manner as in Example 5 except that a compound represented by Formula (UVA-6) was changed to a compound represented by Formula (UVA-9).

### (Preparation Example 1) Preparation of Light-Selective Absorption Composition (A1)

A light-selective absorption composition (A1) was prepared in the same manner as in Example 5 except that a compound represented by Formula (UVA-6) was changed to a compound represented by Formula (UVA-A1).

### (Preparation Example 2) Preparation of Light-Selective Absorption Composition (A2)

A light-selective absorption composition (A2) was prepared in the same manner as in Example 5 except that a compound represented by Formula (UVA-6) was changed to a compound represented by Formula (UVA-A2).

### (Preparation Example 3) Preparation of Light-Selective Absorption Composition (A3)

A light-selective absorption composition (A3) was prepared in the same manner as in Example 5 except that a compound represented by Formula (UVA-6) was changed to a compound represented by Formula (UVA-A4).

### (Example 9) Preparation of Film with Cured Layer (1)

A surface of a resin film [Trade name: “ZEONOR”, produced by Zeon Corporation] made of a cyclic polyolefin resin and having a thickness of 23 μm was subjected to a corona discharge treatment, and the corona discharge-treated surface was coated with the light-selective absorption composition (1) using a bar coater. The coated film was charged into a drying oven and dried at 100° C. for 2 minutes. The dried coating film was charged into a nitrogen replacement box, nitrogen was sealed in the box for 1 minute, and then ultraviolet irradiation was performed from the coated surface side to obtain a film with a cured layer (1). The thickness of the cured layer was about 6.0 μm.

As an ultraviolet irradiation device, an ultraviolet irradiation device with a belt conveyor [a lamp was “H valve” manufactured by Fusion UV Systems, Inc.] was used, and ultraviolet rays were irradiated so that the integrated amount of light was 400 mJ/cm² (UVB).

### (Comparative Example 1) Preparation of Film with Cured Layer (A1)

A film with a cured layer (A1) was obtained in the same manner as in Example 9 except that the light-selective absorption composition (1) was replaced with the light-selective absorption composition (A1).

### (Comparative Example 2) Preparation of Film with Cured Layer (A2)

A film with a cured layer (A2) was obtained in the same manner as in Example 9 except that the light-selective absorption composition (1) was replaced with the light-selective absorption composition (A2).

### (Comparative Example 3) Preparation of Film with Cured Layer (A3)

A film with a cured layer (A3) was obtained in the same manner as in Example 9 except that the light-selective absorption composition (1) was replaced with the light-selective absorption composition (A3).

<Absorbance Measurement of Film with Cured Layer>

The film with a cured layer (1) obtained in Example 9 was cut into a size of 30 mm×30 mm to give a sample (1). The obtained sample (1) and alkali-free glass [trade name “EAGLE XG” produced by Corning Incorporated] were bonded to each other with an acrylic pressure-sensitive adhesive interposed therebetween to obtain a sample (2). The absorbance of the prepared sample (2) in the wavelength range of 300 to 800 nm was measured every 1 nm step using a spectrophotometer (UV-2450: manufactured by Shimadzu Corporation). The measured absorbances at a wavelength of 395 nm and a wavelength of 430 nm were taken as the absorbances at a wavelength of 395 nm and a wavelength of 430 nm of the film with a cured layer (1). The results are shown in Table 1. The absorbance of the alkali-free glass at a wavelength of 395 nm and a wavelength of 430 nm is almost 0, the absorbance of the resin film made of the cyclic polyolefin resin at a wavelength of 395 nm and a wavelength of 430 nm is almost 0, and the absorbance of the acrylic pressure-sensitive adhesive at a wavelength of 395 nm and a wavelength of 430 nm is almost 0.

<Measurement of Absorbance Retention of Film with Cured Layer>

The sample (2) after the absorbance measurement was charged into a Sunshine weathermeter (manufactured by Suga Test Instruments Co., Ltd.) for 48 hours under the conditions of a temperature of 63° C. and a relative humidity of 50% RH to perform a weather resistance test. The absorbance of the sample (2) after the weather resistance test was measured in the same manner as described above. From the measured absorbance, the absorbance retention of the sample (2) at a wavelength of 395 nm was determined based on the following formula. The results are shown in Table 1. A value of the absorbance retention closer to 100 indicates that the light selective absorption function is not deteriorated and good weather resistance is obtained. A (395) represents absorbance at a wavelength of 395 nm.

Absorbance retention (%)=(A(395) after durability test/A(395) before durability test)×100

A film with a cured layer (A1), a film with a cured layer (A2), and a film with a cured layer (A3) were each used instead of the film with a cured layer (1), and evaluation was performed in the same manner as in the film with a cured layer (1). The results are shown in Table 1.

TABLE 1

| | | | | A(395)/ | Absorbance |
|---|---|---|---|---|---|
| | Compounds | A(395) | A(430) | A(430) | retention |
| Example 19 | Formula | 1.24 | 0.03 | 40.0 | 63.9 |
| | (UVA-6) | | | | |
| Comparative | Formula | 2.08 | 0.05 | 40.8 | 4.9 |
| Example 1 | (UVA-A1) | | | | |
| Comparative | Formula | 2.51 | 0.04 | 53.3 | 6.6 |
| Example 2 | (UV1-12) | | | | |
| Comparative | Formula | 1.72 | 0.26 | 6.7 | 35.1 |
| Example 3 | (UVA-14) | | | | |

### (Example 10) Preparation of Optical Film (1)

A resin solution (solid concentration: 25% by mass) containing 70 parts of a polymethyl methacrylate resin (SUMIPEX MH produced by Sumitomo Chemical Co., Ltd.), 30 parts of rubber particles having a particle diameter of 250 nm and having a core-shell structure of polymethyl methacrylate resin (PMMA)/polybutyl acrylate resin (PBA), 2 parts of a compound represented by Formula (UVA-6), and 2-butanone was charged into a mixing tank, and stirred to dissolve the respective components.

The obtained solution was uniformly cast on a glass support using an applicator, dried in an oven at 40° C. for 10 minutes, and then further dried in an oven at 80° C. for 10 minutes. After drying, an optical film (1) was peeled off from the glass support to obtain the optical film (1) having a light-selective absorption capacity. The film thickness of the optical film (1) after drying was 30 μm.

### (Example 11) Preparation of Optical Film (2)

A resin solution (solid concentration: 7% by mass) containing 100 parts of cellulose triacetate (degree of acetyl substitution: 2.87), 2 parts of a compound represented by Formula (UVA-6), and a mixed solution of chloroform and ethanol (mass ratio, chloroform:ethanol=90:10) was charged into a mixing tank, and stirred to dissolve the respective components.

The obtained solution was uniformly cast on a glass support using an applicator, dried in an oven at 40° C. for 10 minutes, and then further dried in an oven at 80° C. for 10 minutes. After drying, an optical film (2) was peeled off from the glass support to obtain the optical film (2) having a light-selective absorption capacity. The film thickness of the optical film (2) after drying was 30 μm.

### (Example 12) Preparation of Optical Film (3)

A resin solution (solid concentration: 20% by mass) containing 100 parts of a cycloolefin polymer resin (ARTON F 4520 produced by JSRCorporation), 2 parts of a compound represented by Formula (UVA-6), and a mixed solution of dichloromethane and toluene (mass ratio, dichloromethane:toluene=50:50) was charged into a mixing tank, and stirred to dissolve each component.

The obtained solution was uniformly cast on a glass support using an applicator, dried in an oven at 40° C. for 10 minutes, and then further dried in an oven at 80° C. for 10 minutes. After drying, an optical film (3) was peeled off from the glass support to obtain the optical film (3) having a light-selective absorption capacity. The film thickness of the optical film (3) after drying was 30 μm.

### (Comparative Example 4) Preparation of Optical Film (4)

An optical film (4) was produced in the same manner as in Example 10 except that the compound represented by Formula (UVA-6) was changed to a compound represented by Formula (OVA-A1).

### (Comparative Example 5) Preparation of Optical Film (5)

An optical film (5) was produced in the same manner as in Example 11 except that the compound represented by Formula (UVA-6) was changed to a compound represented by Formula (UVA-A1).

### (Comparative Example 6) Preparation of Optical Film (6)

An optical film (6) was produced in the same manner as in Example 10 except that the compound represented by Formula (UVA-6) was changed to a compound represented by Formula (UVA-A4).

### (Comparative Example 7) Preparation of Optical Film (7)

An optical film (7) was produced in the same manner as in Example 11 except that the compound represented by Formula (UVA-6) was changed to a compound represented by Formula (UVA-A4).

<Absorbance Measurement of Optical Film>

One surface of the optical film (1) obtained in Example 10 was subjected to a corona discharge treatment, and then an acrylic pressure-sensitive adhesive was bonded thereto by a laminator, and cured for 7 days under conditions of a temperature of 23° C. and a relative humidity of 65% RH to obtain an optical film with a pressure-sensitive adhesive (1). Next, the optical film with a pressure-sensitive adhesive (1) was cut into a size of 30 mm×30 mm and bonded to alkali-free glass [trade name “EAGLE XG” produced by Corning Incorporated] to prepare a sample (3). The absorbance of the prepared sample (3) in the wavelength range of 300 to 800 nm was measured every 1 nm step using a spectrophotometer (UV-2450: manufactured by Shimadzu Corporation). The measured absorbances at a wavelength of 395 nm and a wavelength of 430 nm were taken as the absorbances at a wavelength of 395 nm and a wavelength of 430 nm of the optical film (1). The results are shown in Table 2. The absorbance of the alkali-free glass at a wavelength of 395 nm and a wavelength of 430 nm is almost 0, and the absorbance of the acrylic pressure-sensitive adhesive at a wavelength of 395 nm and a wavelength of 430 nm is almost 0.

The sample (3) after the absorbance measurement was charged into a Sunshine weathermeter (manufactured by Suga Test Instruments Co., Ltd.) for 200 hours under the conditions of a temperature of 63° C. and a relative humidity of 50% RH to perform a weather resistance test. The absorbance of the sample (3) after the weather resistance test was measured in the same manner as described above. From the measured absorbance, the absorbance retention of the sample (3) at a wavelength of 395 nm was determined based on the following formula. The results are shown in Table 2. A value of the absorbance retention closer to 100 indicates that the light selective absorption function is not deteriorated and good weather resistance is obtained.

Absorbance retention (%)=(A(395) after durability test/A(395) before durability test)×100

Each of the optical films (2) to (7) was used instead of the optical film (1), and evaluation was performed in the same manner as in the optical film (1). The results are shown in Table 2.

TABLE 2

| | | | A | A | A(395)/ | Absorbance |
|---|---|---|---|---|---|---|
| | Compounds | Resin | (395) | (430) | A(430) | retention |
| Example | Formula | Polymethyl | 3.01 | 0.03 | 103.8 | 100.0 |
| 10 | (UVA-6) | methacrylate | | | | |
| | | resin | | | | |
| Example | Formula | Cellulose | 3.42 | 0.03 | 126.6 | 95.6 |
| 11 | (UVA-6) | acetate | | | | |
| Example | Formula | Cycloolefin | 3.26 | 0.02 | 163.1 | 76.3 |
| 12 | (UVA-6) | | | | | |
| Compar- | Formula | Polymethyl | 3.56 | 0.02 | 161.9 | 11.3 |
| ative | (UVA-A1) | methacrylate | | | | |
| Example | | resin | | | | |
| 4 | | | | | | |
| Compar- | Formula | Cellulose | 3.64 | 0.04 | 93.4 | 3.4 |
| ative | (UVA-A1) | acetate | | | | |
| Example | | | | | | |
| 5 | | | | | | |
| Compar- | Formula | Polymethyl | 4.04 | 0.56 | 7.2 | 53.8 |
| ative | (UVA-A4) | methacrylate | | | | |
| Example | | resin | | | | |
| 6 | | | | | | |
| Compar- | Formula | Cellulose | 4.22 | 0.74 | 5.7 | 46.6 |
| ative | (UVA-A4) | acetate | | | | |
| Example | | | | | | |
| 7 | | | | | | |

### (Example 13) Preparation of Pressure-Sensitive Adhesive Composition (1)

<Preparation of Acrylic Resin (A)>

In a reaction container equipped with a cooling tube, a nitrogen introduction tube, a thermometer, and a stirrer, a mixed solution of 81.8 parts of ethyl acetate as a solvent, 70.4 parts of butyl acrylate, 20.0 parts of methyl acrylate, 8.0 parts of 2-phenoxyethyl acrylate, 1.0 part of 2-hydroxyethyl acrylate, and 0.6 parts of acrylic acid as monomers were charged, and the internal temperature was raised to 55° C. while air in the reaction container was replaced with nitrogen gas to exclude oxygen. Thereafter, a solution prepared by dissolving 0.14 parts of azobisisobutyronitrile (polymerization initiator) in 10 parts of ethyl acetate was added in its entirety. After the initiator was added, the mixture was kept at this temperature for 1 hour, and then ethyl acetate was continuously added into the reaction container at an addition rate of 17.3 parts/hr while the internal temperature was kept at 54 to 56° C., and when the concentration of the acrylic resin reached 35%, the addition of ethyl acetate was stopped, and the mixture was kept at this temperature until 12 hours had elapsed from the start of addition of ethyl acetate. Finally, ethyl acetate was added to adjust the concentration of the acrylic resin to 20%, thereby preparing an ethyl acetate solution of the acrylic resin. The obtained acrylic resin had a polystyrene equivalent weight average molecular weight Mw of 1.42 million and Mw/Mn of 5.2 as measured by GPC. This is referred to as an acrylic resin (A).

<Preparation of Pressure-Sensitive Adhesive Composition (1)>

To 100 parts of solid content of the ethyl acetate solution (1) (resin concentration: 20%) of the acrylic resin (A) synthesized above, 0.5 parts of a crosslinking agent (ethyl acetate solution of trimethylolpropane adduct of tolylene diisocyanate (solid concentration: 75%), produced by Tosoh Corporation, trade name “Coronate L”), 0.5 parts of a silane compound (3-glycidoxypropyltrimethoxysilane, produced by Shin-Etsu Chemical Co., Ltd., trade name “KBM 403”), and 2.0 parts of a compound represented by Formula (UVA-6) were mixed, and ethyl acetate was further added so that the solid content concentration was 14% to obtain a pressure-sensitive adhesive composition (1). The blending amount of the crosslinking agent is the number of parts by mass as an active component.

### (Example 14) Preparation of Pressure-Sensitive Adhesive Composition (2)

A pressure-sensitive adhesive composition (2) was obtained in the same manner as that in Example 13 except that the compound represented by Formula (UVA-6) was changed to the compound represented by Formula (UVA-7).

### (Example 15) Preparation of Pressure-Sensitive Adhesive Composition (3)

A pressure-sensitive adhesive composition (3) was obtained in the same manner as that in Example 13 except that the compound represented by Formula (UVA-6) was changed to the compound represented by Formula (UVA-8).

### (Example 16) Preparation of Pressure-Sensitive Adhesive Composition (4)

A pressure-sensitive adhesive composition (4) was obtained in the same manner as that in Example 13 except that the compound represented by Formula (UVA-6) was changed to the compound represented by Formula (UVA-9).

### (Comparative Example 8) Preparation of Pressure-Sensitive Adhesive Composition (5)

A pressure-sensitive adhesive composition (5) was obtained in the same manner as that in Example 13 except that the compound represented by Formula (UVA-6) was changed to the compound represented by Formula (UVA-A1).

### (Example 17) Preparation of Pressure-Sensitive Adhesive Layer (1) and Pressure-Sensitive Adhesive Sheet (1)

The obtained pressure-sensitive adhesive composition (1) was applied to a release-treated surface of a separator film [trade name “PLR-382190” available from Lintec Corporation] made of a release-treated polyethylene terephthalate film using an applicator, and dried at 100° C. for 1 minute to prepare a pressure-sensitive adhesive layer (1). The thickness of the obtained pressure-sensitive adhesive layer was 15 μm.

The obtained pressure-sensitive adhesive layer (1) was bonded to a cycloolefin film of 23 μm by a laminator, and then aged for 7 days under conditions of a temperature of 23° C. and a relative humidity of 65% RH to obtain a pressure-sensitive adhesive sheet (1).

### (Example 18) Preparation of Pressure-Sensitive Adhesive Layer (2) and Pressure-Sensitive Adhesive Sheet (2)

A pressure-sensitive adhesive layer (2) and a pressure-sensitive adhesive sheet (2) were prepared in the same manner as in Example 17 except that the pressure-sensitive adhesive composition (1) was changed to the pressure-sensitive adhesive composition (2).

### (Comparative Example 9) Preparation of Pressure-Sensitive Adhesive Layer (3) and Pressure-Sensitive Adhesive Sheet (3)

A pressure-sensitive adhesive layer (3) and a pressure-sensitive adhesive sheet (3) were prepared in the same manner as in Example 17 except that the pressure-sensitive adhesive composition (1) was changed to the pressure-sensitive adhesive composition (5).

<Absorbance Measurement of Pressure-Sensitive Adhesive Sheet>

The obtained pressure-sensitive adhesive sheet (1) was cut into a size of 30 mm×30 mm, the separate film was peeled off, and the pressure-sensitive adhesive layer (1) and alkali-free glass [trade name “EAGLE XG” produced by Corning Incorporated] were bonded to each other to prepare a sample (4). The absorbance of the prepared sample (4) in the wavelength range of 300 to 800 nm was measured every 1 nm step using a spectrophotometer (UV-2450: manufactured by Shimadzu Corporation). The measured absorbances at a wavelength of 395 nm and a wavelength of 430 nm were taken as the absorbances at a wavelength of 395 nm and a wavelength of 430 nm of the pressure-sensitive adhesive sheet (1). The results are shown in Table 3. Both the cycloolefin film alone and the non-alkali glass alone have zero absorbance at a wavelength of 390 nm.

<Measurement of Absorbance Retention of Pressure-Sensitive Adhesive Sheet>

The sample (4) after the absorbance measurement was charged into a Sunshine weathermeter (manufactured by Suga Test Instruments Co., Ltd.) for 200 hours under the conditions of a temperature of 63° C. and a relative humidity of 50% RH to perform a weather resistance test. The absorbance of the sample (4) taken out was measured in the same manner as described above. From the measured absorbance, the absorbance retention of the sample (4) at 395 nm was determined based on the following formula. The results are shown in Table 3. A value of the absorbance retention closer to 100 indicates that the light selective absorption function is not deteriorated and good weather resistance is obtained.

Absorbance retention=(A(395) after durability test/A(395) before durability test)×100

Evaluation was performed in the same manner as in the case of the pressure-sensitive adhesive sheet (1) using each of the pressure-sensitive adhesive sheet (2) and the pressure-sensitive adhesive sheet (3) instead of the pressure-sensitive adhesive sheet (1). The results are shown in Table 3

TABLE 3

| | | | | A(395)/ | Absorbance |
|---|---|---|---|---|---|
| | Compounds | A(395) | A(430) | A(430) | retetion |
| Example 17 | Formula | 1.45 | 0.01 | 111.4 | 100 |
| | (UVA-6) | | | | |
| Example 18 | Formula | 1.26 | 0.03 | 43.3 | 99.4 |
| | (UVA-7) | | | | |
| Comparative | Formula | 2.82 | 0.01 | 216.7 | 6.8 |
| Example 9 | (OVA-Al) | | | | |

### (Example 19) Synthesis of Compound Represented by Formula (UVA-10)

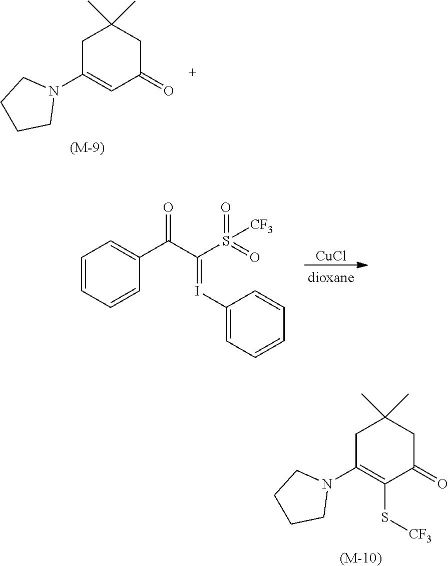

Under a nitrogen atmosphere, 2.5 parts of a compound represented by Formula (M-9), 15.1 parts of benzoyl (phenyliodonium) (trifluoromethanesulfonyl) methanide, 0.4 parts of copper (I) chloride, and 100 parts of dioxane were mixed. The obtained mixture was allowed to stir at 30° C. for 3 hours. The solvent was distilled off from the obtained mixture and purified to obtain 1.7 parts of a compound represented by Formula (M-10).

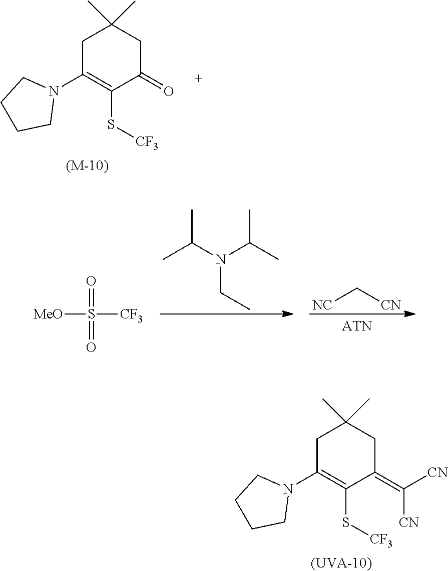

Under a nitrogen atmosphere, 1.5 parts of a compound represented by Formula (M-10), 1.4 parts of methyl triflate, and 10 parts of acetonitrile were mixed, and the mixture was stirred at 20 to 30° C. for 3 hours. 1.3 parts of diisopropylethylamine and 0.7 parts of malononitrile were added to the obtained mixture, and the mixture was stirred at 20 to 30° C. for 3 hours. The solvent was distilled off from the obtained mixture and purified to obtain 1.0 part of a compound represented by Formula (UVA-10).

LC-MS measurement and ¹H-NMR analysis were performed, and it was confirmed that a compound represented by Formula (UVA-10) was produced.

¹H-NMR (deuterated DMSO) δ: 1.00 (s, 3H), 1.15 (s, 3H), 1.86 (m, 2H), 2.18 (m, 2H), 2.32-2.91 (m, 4H), 3.50-4.20 (m, 4H)

LC-MS; [M+H]⁺=343.5

In addition, the maximum absorption wavelength and the gram absorption coefficient were measured in the same manner as described above. The maximum absorption wavelength of the obtained compound represented by Formula (UVA-10) was 384.2 nm. The obtained compound represented by Formula (UVA-10) had ε(λ max) of 1.29 L/(g·cm), ε(λ max+30 nm) of 0.075 L/(g·cm), and ε(max)/ε(λ max+30 nm) of 17.2.

### (Example 20) Synthesis of Compound Represented by Formula (UVA-11)

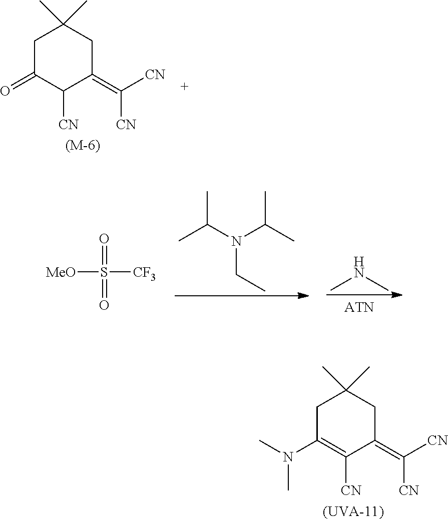

Under a nitrogen atmosphere, 5 parts of a compound represented by Formula (M-6), 4.9 parts of methyl triflate, 3.8 parts of diisopropylethylamine and 10 parts of acetonitrile were mixed, and the mixture was stirred at 20 to 30° C. for 3 hours. 5 parts of dimethylamine was added to the obtained mixture, and the mixture was stirred at 20 to 30° C. for 3 hours. The solvent was distilled off from the obtained mixture and purified to obtain 3.1 parts of a compound represented by Formula (UVA-11).

LC-MS measurement and ¹H-NMR analysis were performed, and it was confirmed that a compound represented by Formula (UVA-11) was produced.

¹H-NMR (deuterated DMSO) δ: 1.08 (s, 6H), 2.42 (s, 2H), 2.55 (s, 2H), 3.40 (m, 6H)

LC-MS; [M+H]⁺=241.5

In addition, the maximum absorption wavelength and the gram absorption coefficient were measured in the same manner as described above. The maximum absorption wavelength of the obtained compound represented by Formula (UVA-11) was 379.4 nm. The obtained compound represented by Formula (UVA-11) had ε(λ max) of 1.93 L/(g·cm), ε(λ max+30 nm) of 0.063 L/(g·cm), and ε(λ max)/ε(λ max+30 nm) of 30.6.

### (Example 21) Synthesis of Compound Represented by Formula (UVA-12)

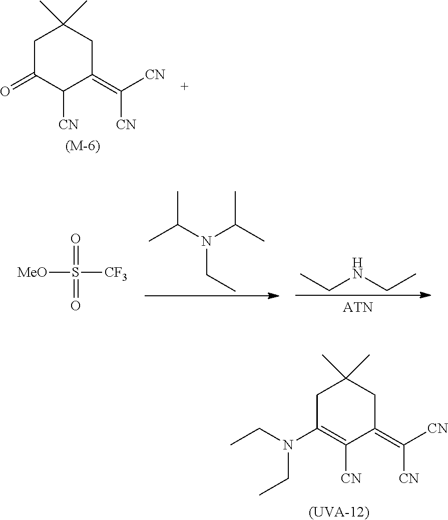

Under a nitrogen atmosphere, 5 parts of a compound represented by Formula (M-6), 4.9 parts of methyl triflate, 3.8 parts of diisopropylethylamine and 10 parts of acetonitrile were mixed, and the mixture was stirred at 20 to 30° C. for 3 hours. 8.4 parts of diethylamine was added to the obtained mixture, and the mixture was stirred at 20 to 30° C. for 3 hours. The solvent was distilled off from the obtained mixture and purified to obtain 2.9 parts of a compound represented by Formula (UVA-12).

LC-MS measurement and ¹H-NMR analysis were performed, and it was confirmed that a compound represented by Formula (UVA-12) was produced.

¹H-NMR (deuterated DMSO) δ: 1.08 (s, 6H), 1.39 (t, 6H), 2.44 (s, 2H), 2.58 (s, 2H), 3.74 (m, 4H)

LC-MS; [M+H]⁺=269.5

in addition, the maximum absorption wavelength and the gram absorption coefficient were measured in the same manner as described above. The maximum absorption wavelength of the obtained compound represented by Formula (UVA-12) was 380.5 nm. The obtained compound represented by Formula (UVA-12) had ε(λ max) of 1.75 L/(g·cm), ε(λ max+30 nm) of 0.098 L/(g·cm), and ε(λ max)/ε(λ max+30 nm) of 17.6.

### (Example 22) Synthesis of Compound Represented by Formula (UVA-13)

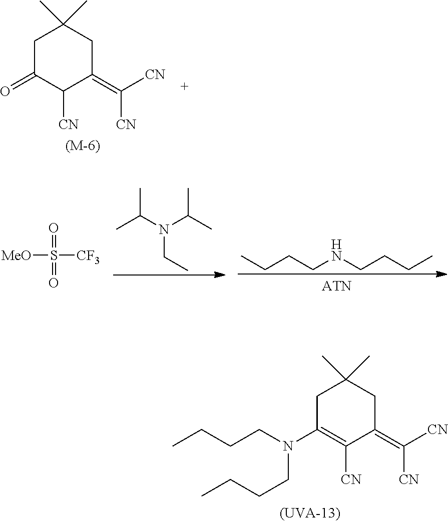

Under a nitrogen atmosphere, 5 parts of a compound represented by Formula (M-6), 4.9 parts of methyl triflate, 3.8 parts of diisopropylethylamine and 10 parts of acetonitrile were mixed, and the mixture was stirred at 20 to 30° C. for 3 hours. 14.8 parts of dibutylamine was added to the obtained mixture, and the mixture was stirred at 20 to 30° C. for 3 hours. The solvent was distilled off from the obtained mixture and purified to obtain 2.5 parts of a compound represented by Formula (UVA-13).

LC-MS measurement and ¹H-NMR analysis were performed, and it was confirmed that a compound represented by Formula (UVA-13) was produced.

¹H-NMR (deuterated DMSO) δ: 0.99 (t, 6H), 1.07 (s, 6H), 1.32 to 1.46 (m, 4H), 1.70 (m, 4H), 2.40 (s, 2H), 2.57 (s, 2H), 3.32 to 3.85 (m, 4H).

LC-MS; [M+H]⁺=325.5

In addition, the maximum absorption wavelength and the gram absorption coefficient were measured in the same manner as described above. The maximum absorption wavelength of the obtained compound represented by Formula (UVA-13) was 382.8 nm. The obtained compound represented by Formula (UVA-13) had ε(λ max) of 1.42 L/(g·cm), ε(λ max+30 nm) of 0.095 L/(g·cm), and ε(λ max)/ε(λ max+30 nm) of 14.9.

### (Example 23) Synthesis of Compound Represented by Formula (UVA-14)

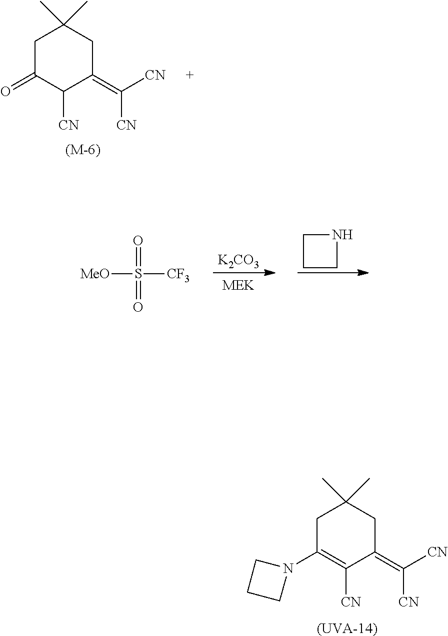

Under a nitrogen atmosphere, 5 parts of a compound represented by Formula (M-6), 3.6 parts of potassium carbonate, 7.7 parts of methyl triflate and 40 parts of methyl ethyl ketone were mixed, and the mixture was stirred at 0 to 5° C. for 4 hours. 2 parts of azetidine was added to the obtained mixture, and the mixture was stirred at 0 to 5° C. for 10 minutes. The solvent was distilled off from the obtained mixture and purified to obtain 2.6 parts of a compound represented by Formula (UVA-14).

LC-MS measurement and ¹H-NMR analysis were performed, and it was confirmed that a compound represented by Formula (UVA-14) was produced.

¹H-NMR (deuterated DMSO) δ: 1.05 (s, 6H), 2.14 (s, 2H), 2.45-2.53 (m, 4H), 4.36 (t, 2H), 4.91 (t, 2H)

LC-MS; [M+H]⁺=253.3

In addition, the maximum absorption wavelength and the gram absorption coefficient were measured in the same manner as described above. The maximum absorption wavelength of the obtained compound represented by Formula (UVA-14) was 377.2 nm. The obtained compound represented by Formula (UVA-14) had ε(λ max) of 1.93 L/(g·cm), ε(λ max+30 nm) of 0.028 L/(g·cm), and ε(λ max)/ε(λ max+30 nm) of 68.9.

### (Example 24) Synthesis of Compound Represented by Formula (UVA-15)

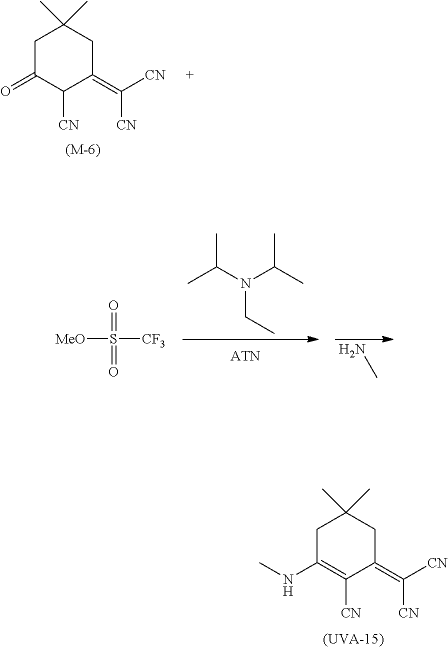

Under a nitrogen atmosphere, 4.0 parts of a compound represented by Formula (M-6), 3.7 parts of methyl triflate, and 40 parts of acetonitrile were mixed, and the mixture was stirred at 20 to 30° C. for 3 hours. To the obtained mixture, 2.9 parts of diisopropylethylamine and 40 parts of a solution obtained by dissolving methylamine in tetrahydrofuran (concentration of methylamine; 7% by mass) was added, and the mixture was stirred at 20 to 30° C. for 3 hours. The solvent was distilled off from the obtained mixture and purified to obtain 1.9 parts of a compound represented by Formula (UVA-15).

LC-MS measurement and ¹H-NMR analysis were performed, and it was confirmed that a compound represented by Formula (UVA-15) was produced.

¹H-NMR (deuterated DMSO) δ: 0.98 (s, 6H), 2.48 to 2.58 (m, 4H), 3.03 (s, 3H), 9.15 (s, 1H)

LC-MS; [M+H]⁺=226.5

In addition, the maximum absorption wavelength and the gram absorption coefficient were measured in the same manner as described above. The maximum absorption wavelength of the obtained compound represented by Formula (UVA-15) was 364.8 nm. The obtained compound represented by Formula (UVA-15) had ε(λ max) of 1.86 L/(g·cm), ε(λ max+30 nm) of 0.066 L/(g·cm), and ε(λ max)/ε(λ max+30 nm) of 28.2.

### (Example 25) Synthesis of Compound Represented by Formula (UVA-16)

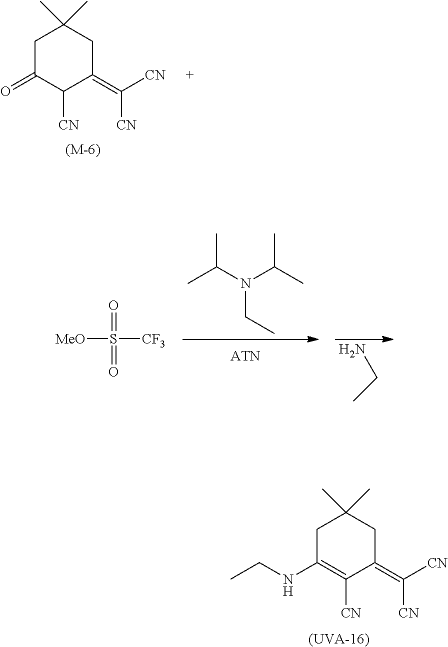

Under a nitrogen atmosphere, 4.0 parts of a compound represented by Formula (M-6), 3.7 parts of methyl triflate, and 40 parts of acetonitrile were mixed, and the mixture was stirred at 20 to 30° C. for 3 hours. To the obtained mixture, 2.9 parts of diisopropylethylamine and 40 parts of a solution obtained by dissolving ethylamine in tetrahydrofuran (concentration of ethylamine; 10% by mass) was added, and the mixture was stirred at 20 to 30° C. for 3 hours. The solvent was distilled off from the obtained mixture and purified to obtain 1.5 parts of a compound represented by Formula (UVA-16).

LC-MS measurement and ¹H-NMR analysis were performed, and it was confirmed that a compound represented by Formula (UVA-16) was produced.

¹H-NMR (deuterated DMSO) δ: 0.98 (s, 6H), 2.48-2.58 (m, 4H), 3.03 (t, 3H), 4.21 (m, 2H), 9.15 (s, 1H)

LC-MS; [M+H]⁺=240.5

In addition, the maximum absorption wavelength and the gram absorption coefficient were measured in the same manner as described above. The maximum absorption wavelength of the obtained compound represented by Formula (UVA-16) was 364.8 nm. The obtained compound represented by Formula (UVA-16) had ε(λ max) of 1.80 L/(g·cm), ε(λ max+30 nm) of 0.074 L/(g·cm), and ε(λ max)/ε(λ max+30 nm) of 24.4.

(Example 26) Synthesis of Compound Represented by Formula (UVA-17)

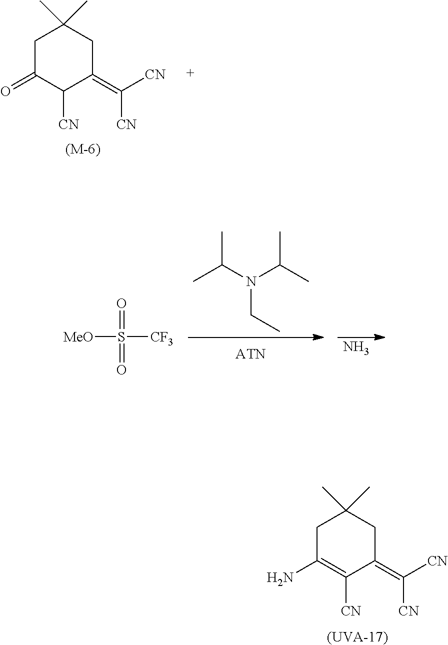

Under a nitrogen atmosphere, 1.7 parts of a compound represented by Formula (M-6), 1.6 parts of methyl triflate, and 17 parts of acetonitrile were mixed, and the mixture was stirred at 20 to 30° C. for 3 hours. To the obtained mixture, 1.2 parts of diisopropylethylamine and 100 parts of a solution obtained by dissolving ammonia in tetrahydrofuran (molar concentration of ammonia; 0.4 mol %) and stirred at 20 to 30° C. for 3 hours. The solvent was distilled off from the obtained mixture and purified to obtain 0.7 parts of a compound represented by Formula (UVA-17).

LC-MS measurement and ¹H-NMR analysis were performed, and it was confirmed that a compound represented by Formula (UVA-17) was produced.

¹H-NMR (deuterated DMSO) δ: 0.98 (s, 6H), 2.48-2.58 (m, 4H), 9.15 (m, 2H)

LC-MS; [M+H]⁺=213.5

In addition, the maximum absorption wavelength and the gram absorption coefficient were measured in the same manner as described above. The maximum absorption wavelength of the obtained compound represented by Formula (UVA-17) was 352.6 nm. The obtained compound represented by Formula (UVA-17) had £(λ max) of 1.75 L/(g·cm), ε(λ max+30 nm) of 0.11 L/(g·cm), and ε(λ max)/ε(λ max+30 nm) of 15.9.

### (Example 27) Preparation of Light-Selective Absorption Composition (5)

A light-selective absorption composition (5) was prepared in the same manner as in Example 5 except that a compound represented by Formula (UVA-6) was changed to a compound represented by Formula (UVA-10).

### (Example 28) Preparation of Light-Selective Absorption Composition (6)

A light-selective absorption composition (6) was prepared in the same manner as in Example 5 except that a compound represented by Formula (UVA-6) was changed to a compound represented by Formula (UVA-11).

### (Example 29) Preparation of Light-Selective Absorption Composition (7)

A light-selective absorption composition (7) was prepared in the same manner as in Example 5 except that a compound represented by Formula (UVA-6) was changed to a compound represented by Formula (UVA-12).

### (Example 30) Preparation of Light-Selective Absorption Composition (8)

A light-selective absorption composition (7) was prepared in the same manner as in Example 5 except that a compound represented by Formula (UVA-6) was changed to a compound represented by Formula (UVA-13).

### (Example 31) Preparation of Film with Cured Layer (2)

A film with a cured layer (2) was obtained in the same manner as in Example 9 except that the light-selective absorption composition (1) was replaced with the light-selective absorption composition (6).

### (Example 32) Preparation of Film with Cured Layer (3)

A film with a cured layer (3) was obtained in the same manner as in Example 9 except that the light-selective absorption composition (1) was replaced with the light-selective absorption composition (7).

<Absorbance Measurement and Absorbance Retention Measurement of Film with Cured Laver>

The absorbance was measured in the same manner as in <Absorbance Measurement of Film with Cured Layer> described above except that the film with a cured layer (2) and the film with a cured layer (3) were used instead of the film with a cured layer (1).

In addition, the absorbance retention of the film with a cured layer (1) obtained in Example 9 and the film with a cured layer (A3) obtained in Comparative Example 3 were measured in the same manner as in <Measurement of Absorbance Retention of Film with Cured Layer> described above except that the charging time to the sunshine weathermeter was set to 75 hours.

Further, the absorbance retention was measured in the same manner as in <Measurement of Absorbance Retention of Film with Cured Layer> described above except that the film with a cured layer (2) and the film with a cured layer (3) were used instead of the film with a cured layer (1), and the charging time to the sunshine weathermeter was set to 75 hours.

These results are shown in Table 4. Table 4 also shows the absorbance values of the film with a cured layer (1) obtained in Example 9 and the film with a cured layer (A3) obtained in Comparative Example 3.

TABLE 4

| | | | | A(395)/ | Absorbance |
|---|---|---|---|---|---|
| | Compounds | A(395) | A(430) | A(430) | retention |
| Example 31 | Formula | 1.199 | 0.044 | 27.3 | 56 |
| | (WA-11) | | | | |
| Example 32 | Formula | 1.163 | 0.022 | 52.9 | 54 |
| | (UVA-12) | | | | |
| Example 9 | Formula | 1.24 | 0.03 | 40.0 | 39.8 |
| | (UVA-6) | | | | |
| Comparative | Formula | 1.72 | 0.26 | 6.7 | 7.1 |
| Example 3 | (UVA-A4) | | | | |

### (Example 33) Preparation of Pressure-Sensitive Adhesive Composition (6)

A pressure-sensitive adhesive composition (6) was obtained in the same manner as that in Example 13 except that the compound represented by Formula (UVA-6) was changed to the compound represented by Formula (UVA-10).

### (Example 34) Preparation of Pressure-Sensitive Adhesive Composition (7)

A pressure-sensitive adhesive composition (7) was obtained in the same manner as that in Example 13 except that the compound represented by Formula (UVA-6) was changed to the compound represented by Formula (UVA-11).

### (Example 35) Preparation of Pressure-Sensitive Adhesive Composition (8)

A pressure-sensitive adhesive composition (8) was obtained in the same manner as that in Example 13 except that the compound represented by Formula (UVA-6) was changed to the compound represented by Formula (UVA-12).

### (Example 36) Preparation of Pressure-Sensitive Adhesive Composition (9)

A pressure-sensitive adhesive composition (9) was obtained in the same manner as that in Example 13 except that the compound represented by Formula (UVA-6) was changed to the compound represented by Formula (UVA-13).

### (Example 37) Preparation of Pressure-Sensitive Adhesive Layer (4) and Pressure-Sensitive Adhesive Sheet (4)

A pressure-sensitive adhesive layer (4) and a pressure-sensitive adhesive sheet (4) were prepared in the same manner as in Example 17 except that the pressure-sensitive adhesive composition (1) was changed to the pressure-sensitive adhesive composition (4).

### (Example 38) Preparation of Pressure-Sensitive Adhesive Layer (5) and Pressure-Sensitive Adhesive Sheet (5)

A pressure-sensitive adhesive layer (5) and a pressure-sensitive adhesive sheet (5) were prepared in the same manner as in Example 17 except that the pressure-sensitive adhesive composition (1) was changed to the pressure-sensitive adhesive composition (6).

### (Example 39) Preparation of Pressure-Sensitive Adhesive Layer (6) and Pressure-Sensitive Adhesive Sheet (6)

A pressure-sensitive adhesive layer (6) and a pressure-sensitive adhesive sheet (6) were prepared in the same manner as in Example 17 except that the pressure-sensitive adhesive composition (1) was changed to the pressure-sensitive adhesive composition (7).

### (Example 40) Preparation of Pressure-Sensitive Adhesive Layer (7) and Pressure-Sensitive Adhesive Sheet (7)

A pressure-sensitive adhesive layer (7) and a pressure-sensitive adhesive sheet (7) were prepared in the same manner as in Example 17 except that the pressure-sensitive adhesive composition (1) was changed to the pressure-sensitive adhesive composition (8).

### (Example 41) Preparation of Pressure-Sensitive Adhesive Layer (8) and Pressure-Sensitive Adhesive Sheet (8)

A pressure-sensitive adhesive layer (8) and a pressure-sensitive adhesive sheet (8) were prepared in the same manner as in Example 17 except that the pressure-sensitive adhesive composition (1) was changed to the pressure-sensitive adhesive composition (9).

<Absorbance Measurement and Absorbance Retention Measurement of Pressure-Sensitive Adhesive Sheet>

The absorbance and the absorbance retention were measured in the same manner as in <Measurement of Absorbance of Pressure-Sensitive Adhesive Sheet> and <Measurement of Absorbance Retention of Pressure-Sensitive Adhesive Sheet> described above except that the pressure-sensitive adhesive sheet (4) to the pressure-sensitive adhesive sheet (8) were used instead of the pressure-sensitive adhesive sheet (1). The results are shown in Table 5.

| | | | | A(395)/ | Absorbance |
|---|---|---|---|---|---|
| | Compound | A (395) | A(430) | A(430) | retention |
| Example 37 | Formula | 3.18 | 0.025 | 127.4 | 96.2 |
| | (UVA-9) | | | | |
| Example 38 | Formula | 2.35 | 0.031 | 75.9 | 84.8 |
| | (1JVA-10) | | | | |
| Example 39 | Formula | 2.02 | 0.009 | 224.3 | 98.9 |
| | (OVA-Il) | | | | |
| Example 40 | Formula | 2.29 | 0.014 | 163.9 | 98.2 |
| | (WA-12) | | | | |
| Example 41 | Formula | 0.97 | 0.001 | 974.0 | 85.8 |
| | (UVA-13) | | | | |

### (Example 42) Synthesis of Compound Represented by Formula (UVA-20)

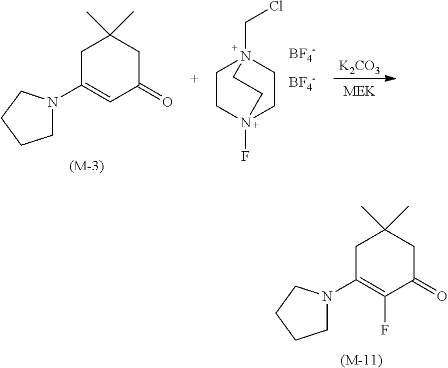

Under a nitrogen atmosphere, 17 parts of a compound represented by Formula (M-3), 12.2 parts of potassium carbonate, 15.9 parts of 1-chloromethyl-4 fluoro-1,4-diazoniabicyclo [2.2.2.] octane bis(tetrafluoroborate) (selectfluoro, registered trademark of Air Products and Chemicals), and 85 parts of methyl ethyl ketone were mixed, and the mixture was stirred in an ice bath for 3 hours. The solvent was distilled off from the obtained mixture and purified to obtain 3.7 parts of a compound represented by Formula (M-11).

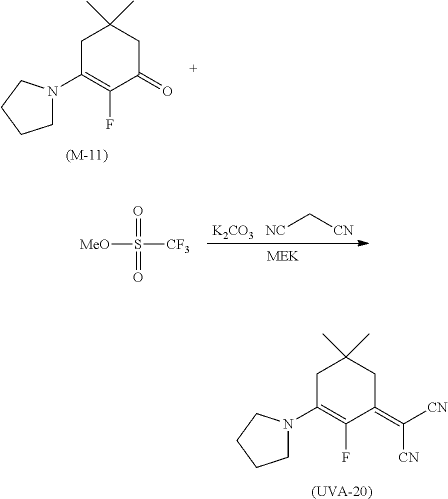

Under a nitrogen atmosphere, 18 parts of a compound represented by Formula (M-11), 28 parts of methyl triflate and 90 parts of methyl ethyl ketone were mixed, and the mixture was stirred at 20 to 30° C. for 3 hours. 13.0 parts of potassium carbonate and 8.4 parts of malononitrile were added to the obtained mixture, and the mixture was stirred at 20 to 30° C. for 3 hours. The solvent was distilled off from the obtained mixture and purified to obtain 5.8 parts of a compound represented by Formula (UVA-20).

LC-MS measurement and ¹H-NMR analysis were performed, and it was confirmed that a compound represented by Formula (UVA-20) was produced.

¹H-NMR (deuterated DMSO) δ: 1.08 (s, 6H), 1.97 (m, 4H), 2.40 (d, 2H), 2.50 (d, 2H), 3.53 (m, 2H), 3.86 (m, 2H)

LC-MS; [M+H]⁺=260.5

In addition, the maximum absorption wavelength and the gram absorption coefficient were measured in the same manner as described above. The maximum absorption wavelength of the obtained compound represented by Formula (UVA-20) was 407.5 nm. The obtained compound represented by Formula (UVA-20) had ε(λ max) of 2.30 L/(g·cm), ε(λ max+30 nm) of 0.041 L/(g·cm), and ε(λ max)/ε(λ max+30 nm) of 56.0.

### (Example 43) Synthesis of Compound Represented by Formula (UVA-21)

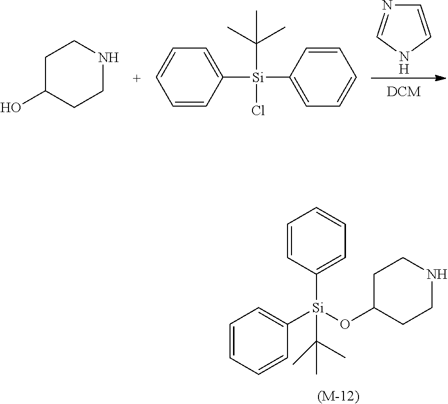

Under a nitrogen atmosphere, 5 parts of 3-hydroxypiperidine, 13.6 parts of tertiary-butyldiphenylsilyl chloride, 6.7 parts of imidazole, and 40 parts of dichloromethane were mixed, and the mixture was stirred at 20 to 30° C. for 4 hours. The solvent was distilled off from the obtained mixture and purified to obtain 10.5 parts of a compound represented by Formula (M-12).

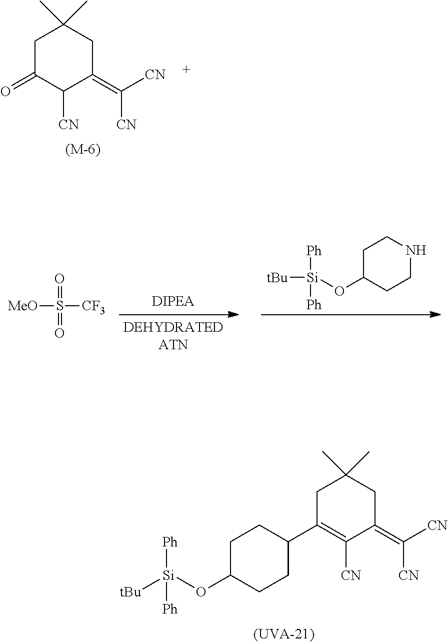

Under a nitrogen atmosphere, 4.0 parts of a compound represented by Formula (M-6), 3.2 parts of diisopropylethylamine, 4.0 parts of methyl triflate, and 80 parts of acetonitrile were mixed, and the mixture was stirred at 20 to 30° C. for 4 hours. 8.3 parts of a compound represented by Formula (51-12) was added to the obtained mixture, and the mixture was stirred at 20 to 30° C. for 3 hours. The solvent was distilled off from the obtained mixture and purified to obtain 6.5 parts of a compound represented by Formula (UVA-21).

LC-MS measurement and ¹H-NMR analysis were performed, and it was confirmed that a compound represented by Formula (UVA-21) was produced.

¹H-NMR (deuterated DMSO) δ: 0.97 (s, 6H), 1.04 (s, 9H), 1.70 (m, 2H), 1.85 (m, 2H), 2.48 (s, 2H), 2.65 (s, 2H), 3.72 (m, 2H), 3.94 (m, 2H), 4.13 (m, 1H), 7.42-7.52 (m, 6H), 7.61-7.64 (m, 4H)

LC-MS; [M+H]⁺=535.9

### (Example 44) Synthesis of Compound Represented by Formula (UVA-22)

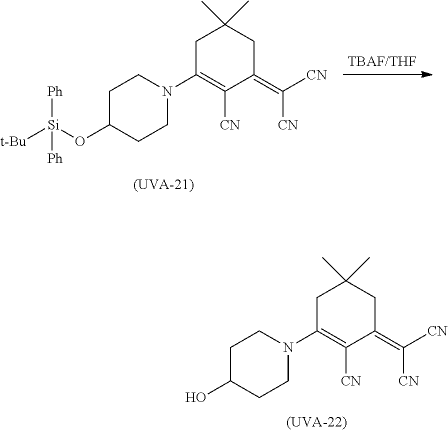

Under a nitrogen atmosphere, 4.2 parts of a compound represented by Formula (UVA-21) and 50 parts of a 1 M solution of tetrabutylammonium fluoride/tetrahydrofuran were mixed, and the mixture was stirred at 20 to 30° C. for 40 hours. The solvent was distilled off from the obtained mixture and purified to obtain 1.8 parts of a compound represented by Formula (UVA-22).

LC-MS measurement and ¹H-NMR analysis were performed, and it was confirmed that a compound represented by Formula (UVA-22) was produced.

¹H-NMR (deuterated DMSO) δ: 0.98 (s, 6H), 1.59 (m, 2H), 1.92 (m, 2H), 2.67 (s, 2H), 3.68-3.95 (m, 4H), 4.97 (m, 1H)

LC-MS; [M+H]⁺=297.5

In addition, the maximum absorption wavelength and the gram absorption coefficient were measured in the same manner as described above. The maximum absorption wavelength of the obtained compound represented by Formula (UVA-22) was 384.6 nm. The obtained compound represented by Formula (UVA-22) had ε(λ max) of 1.43 L/(g·cm), ε(λ max+30 nm) of 0.085 L/(g·cm), and ε(λ max)/ε(λ max+30 nm) of 16.8.

### (Example 45) Synthesis of Compound Represented by Formula (UVA-23)

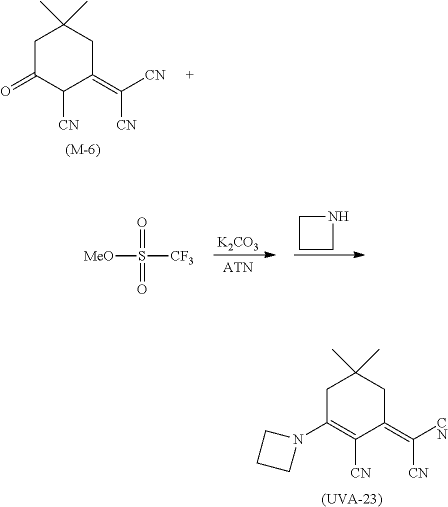

Under a nitrogen atmosphere, 5.0 parts of a compound represented by Formula (M-6), 3.6 parts of potassium carbonate, 7.7 parts of methyl triflate, and 40 parts of acetonitrile were mixed, and the mixture was stirred at 20 to 30° C. for 4 hours. 2.0 parts of azetidine was added to the obtained mixture, and the mixture was stirred at 20 to 30° C. for 4 hours. The solvent was distilled off from the obtained mixture and purified to obtain 2.3 parts of a compound represented by Formula (UVA-23).

LC-MS measurement and ¹H-NMR analysis were performed, and it was confirmed that a compound represented by Formula (UVA-23) was produced.

¹H-NMR (deuterated DMSO) δ: 1.05 (s, 6H), 2.14 (s, 2H), 2.44-2.53 (m, 4H), 4.36 (t, 2H), 4.91 (t, 2H)

LC-MS; [M+H]⁺=253.5

In addition, the maximum absorption wavelength and the gram absorption coefficient were measured in the same manner as described above. The maximum absorption wavelength of the obtained compound represented by Formula (UVA-23) was 377.2 nm. The obtained compound represented by Formula (UVA-23) had ε(λ max) of 1.93 L/(g·cm), ε(λ max+30 nm) of 0.028 L/(g·cm), and ε(λ max)/ε(λ max+30 nm) of 68.9.

### (Example 46) Synthesis of Compound Represented by Formula (UVA-26)

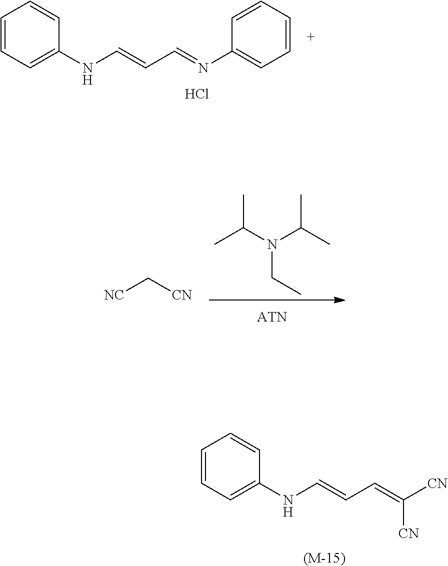

The inside of a 500 mL-four-necked flask equipped with a Dimroth cooling tube and a thermometer was made into a nitrogen atmosphere, 40 parts of malonaldehyde dianilide hydrochloride, 22 parts of diisopropylamine, 200 parts of acetonitrile, and 11 parts of malononitrile were added thereto, and the mixture was stirred in an ice bath for 5 hours. The solvent was distilled off from the obtained mixture and purification was performed to obtain 29 parts of a compound represented by Formula (M-15).

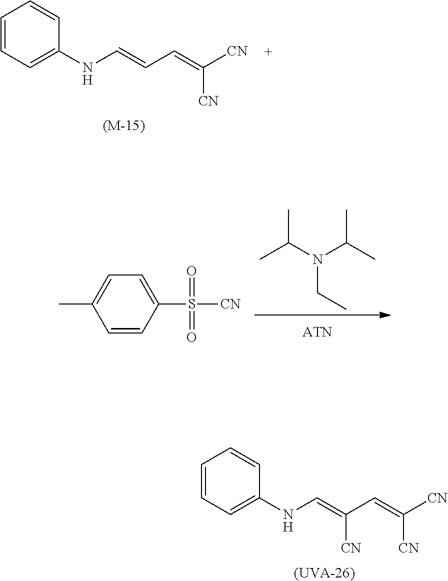

Under a nitrogen atmosphere, 7 parts of the obtained compound represented by Formula (M-15), 5 parts of diisopropylethylamine, 56 parts of acetonitrile, and 7 parts of p-toluenesulfonyl cyanide were mixed, and the mixture was stirred under reflux at 80° C. for 3 hours. The solvent was distilled off from the obtained mixture and purified to obtain 0.1 parts of a compound represented by Formula (UVA-26).

LC-MS measurement was performed, and it was confirmed that a compound represented by Formula (UVA-26) was produced.

LC-MS; [M+H]⁺=221.5

### (Synthesis Example 5) Synthesis of Compound Represented by Formula (UVA-A5)

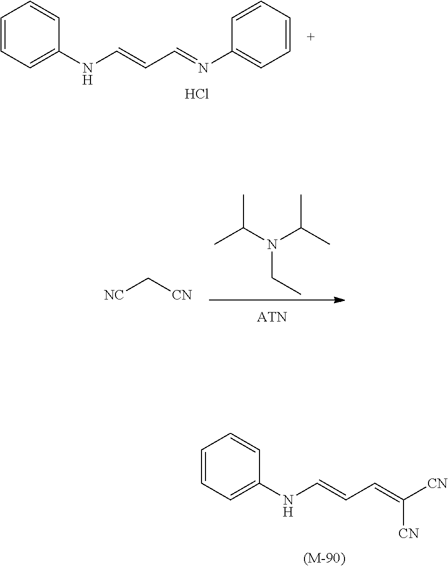

Under a nitrogen atmosphere, 40 parts of malonaldehyde dianilide hydrochloride, 10.7 parts of malononitrile, and 200 parts of acetonitrile were mixed, 22 parts of diisopropylethylamine was added dropwise while stirring in an ice bath, and the mixture was stirred for 4 hours. The solvent was distilled off from the obtained mixture and purified to obtain 28 parts of a compound represented by Formula (M-90).

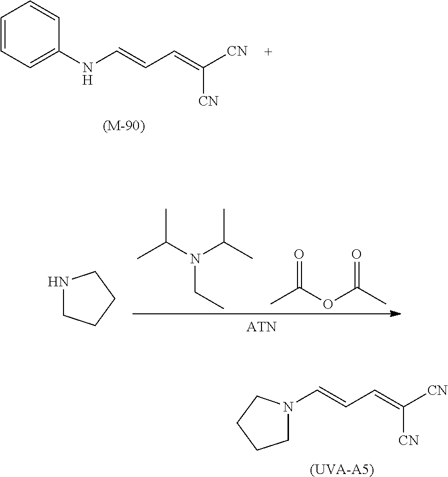

Under a nitrogen atmosphere, 5.0 parts of a compound represented by Formula (M-90), 2.9 parts of acetic anhydride, 6.6 parts of diisopropylethylamine, and 40 parts of acetonitrile were mixed, and the mixture was stirred at 20 to 30° C. for 3 hours. 2.7 parts of pyrrolidine was added to the obtained mixture, the mixture was further stirred for 1 hour, and the solvent was distilled off from the obtained mixture and purified to obtain 2.6 parts of a compound represented by Formula (UVA-A5).

LC-MS measurement and ¹H-NMR analysis were performed, and it was confirmed that a compound represented by Formula (UVA-A5) was produced.

¹H-NMR (deuterated DMSO) δ: 1.83-2.00 (m, 43), 3.37-3.40 (m, 2H), 3.61-3.65 (t, 2H), 5.39-5.45 (t, 13), 7.55-7.58 (d, 1H), 7.84-7.87 (d, 1H)

LC-MS; [M+H]⁺=174.5

In addition, the maximum absorption wavelength and the gram absorption coefficient were measured in the same manner as described above. The maximum absorption wavelength of the obtained compound represented by Formula (UVA-A5) was 379.3 nm. The obtained compound represented by Formula (UVA-A5) had ε(λ max) of 3.41 L/(g·cm), ε(λ max+30 nm) of 0.10 L/(g·cm), and ε(λ max)/ε(λ max+30 nm) of 33.5.

### (Synthesis Example 6) Synthesis of Compound Represented by Formula (UVA-A6)

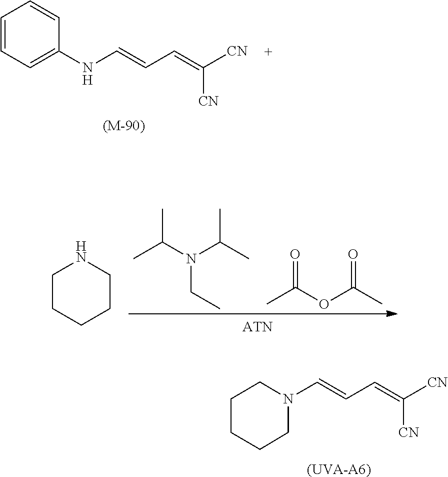

Under a nitrogen atmosphere, 5.0 parts of a compound represented by Formula (M-90), 2.9 parts of acetic anhydride, 6.6 parts of diisopropylethylamine, and 40 parts of acetonitrile were mixed, and the mixture was stirred at 20 to 30° C. for 3 hours. 3.0 parts of piperidine was added to the obtained mixture, the mixture was further stirred for 1 hour, and the solvent was distilled off from the obtained mixture and purified to obtain 2.7 parts of a compound represented by Formula (UVA-A6).

LC-MS measurement and ¹H-NMR analysis were performed, and it was confirmed that a compound represented by Formula (UVA-A6) was produced.

¹H-NMR (deuterated DMSO) δ: 1.72 (m, 6H), 3.40-3.44 (m, 4H), 5.44-5.70 (m, 1H) 6.98-7.00 (d, 1H), 7.19-7.25 (m, 1H)

LC-MS; [M+H]⁺=188.5

In addition, the maximum absorption wavelength and the gram absorption coefficient were measured in the same manner as described above. The maximum absorption wavelength of the obtained compound represented by Formula (UVA-A6) was 374.7 nm. The obtained compound represented by Formula (UVA-A6) had ε(λ max) of 2.89 L/(g·cm), ε(λ max+30 nm) of 0.14 L/(g·cm), and ε(λ max)/ε(λ max+30 nm) of 20.6.

### (Example 47) Preparation of Pressure-Sensitive Adhesive Composition (10)

A pressure-sensitive adhesive composition (10) was obtained in the same manner as that in Example 13 except that the compound represented by Formula (UVA-6) was changed to the compound represented by Formula (UVA-23), and the content of the compound was 0.5 parts based on 100 parts of the acrylic resin (A).

### (Comparative Example 10) Preparation of Pressure-Sensitive Adhesive Composition (11)

A pressure-sensitive adhesive composition (11) was obtained in the same manner as that in Example 13 except that the compound represented by Formula (UVA-6) was changed to the compound represented by Formula (UVA-A5), and the content of the compound was 1.5 parts based on 100 parts of the acrylic resin (A).

### (Comparative Example 11) Preparation of Pressure-Sensitive Adhesive Composition (12)

A pressure-sensitive adhesive composition (12) was obtained in the same manner as that in Example 13 except that the compound represented by Formula (UVA-6) was changed to the compound represented by Formula (UVA-A6), and the content of the compound was 1.5 parts based on 100 parts of the acrylic resin (A).

### (Example 48) Preparation of Pressure-Sensitive Adhesive Layer (9) and Pressure-Sensitive Adhesive Sheet (9)

A pressure-sensitive adhesive layer (9) and a pressure-sensitive adhesive sheet (9) were prepared in the same manner as in Example 17 except that the pressure-sensitive adhesive composition (1) was changed to the pressure-sensitive adhesive composition (10).

### (Comparative Example 12) Preparation of Pressure-Sensitive Adhesive Layer (10) and Pressure-Sensitive Adhesive Sheet (10)

A pressure-sensitive adhesive layer (10) and a pressure-sensitive adhesive sheet (10) were prepared in the same manner as in Example 17 except that the pressure-sensitive adhesive composition (1) was changed to the pressure-sensitive adhesive composition (11).

### (Comparative Example 13) Preparation of Pressure-Sensitive Adhesive Layer (11) and Pressure-Sensitive Adhesive Sheet (11)

A pressure-sensitive adhesive layer (11) and a pressure-sensitive adhesive sheet (11) were prepared in the same manner as in Example 17 except that the pressure-sensitive adhesive composition (1) was changed to the pressure-sensitive adhesive composition (12).

<Absorbance Measurement and Absorbance Retention Measurement of Pressure-Sensitive Adhesive Sheet>

The absorbance and the absorbance retention were measured in the same manner as in <Measurement of Absorbance of Pressure-Sensitive Adhesive Sheet> and <Measurement of Absorbance Retention of Pressure-Sensitive Adhesive Sheet> described above except that the pressure-sensitive adhesive sheet (9) to the pressure-sensitive adhesive sheet (11) were used instead of the pressure-sensitive adhesive sheet (1). The results are shown in Table 6.

| | | | | A(395)/ | Absorbance |
|---|---|---|---|---|---|
| | Compounds | A(395) | A(430) | A(430) | retention |
| Example 48 | Formula | 0.78 | 0.001 | 778.0 | 99.6 |
| | (UVA-23) | | | | |
| Comparative | Formula | 3.385 | 0.01 | 338.5 | 0.9 |
| Example 12 | (CPA-AS) | | | | |
| Comparative | Formula | 2.639 | 0.029 | 91.0 | 1.5 |
| Example 13 | (UVA-A6) | | | | |

### (Example 49) Preparation of Pressure-Sensitive Adhesive Composition (13)

<Preparation of Acrylic Resin (A-2)>

In a reaction container equipped with a cooling tube, a nitrogen introduction tube, a thermometer, and a stirrer, a mixed solution of 81.8 parts of ethyl acetate as a solvent, 96 parts of butyl acrylate, 3 parts of 2-hydroxyethyl acrylate, and 1 part of acrylic acid as monomers were charged, and the internal temperature was raised to 55° C. while air in the reaction container was replaced with nitrogen gas to exclude oxygen. Thereafter, a solution prepared by dissolving 0.14 parts of azobisisobutyronitrile (polymerization initiator) in 10 parts of ethyl acetate was added in its entirety. After the initiator was added, the mixture was kept at this temperature for 1 hour, and then ethyl acetate was continuously added into the reaction container at an addition rate of 17.3 parts/hr while the internal temperature was kept at 54 to 56° C., and when the concentration of the acrylic resin reached 35%, the addition of ethyl acetate was stopped, and the mixture was kept at this temperature until 12 hours had elapsed from the start of addition of ethyl acetate. Finally, ethyl acetate was added to adjust the concentration of the acrylic resin to 20%, thereby preparing an ethyl acetate solution of the acrylic resin. The obtained acrylic resin had a polystyrene equivalent weight average molecular weight Mw of 1.40 million as measured by GPC. The Mw/Mn was 4.8. This is referred to as an acrylic resin (A-2).

<Preparation of Pressure-Sensitive Adhesive Composition (13)>

To 100 parts of solid content of the ethyl acetate solution (resin concentration: 20%) of the acrylic resin (A-2) synthesized above, 0.5 parts of a crosslinking agent (ethyl acetate solution of trimethylolpropane adduct of tolylene diisocyanate (solid concentration: 75%), produced by Tosoh Corporation, trade name “Coronate L”), 0.3 parts of a silane compound (1,6-bis(trimethoxysilyl) hexane, produced by Shin-Etsu Chemical Co., Ltd., trade name “KBM 3066”), and 3 parts of a compound represented by Formula (UVA-6) were mixed, and ethyl acetate was further added so that the solid content concentration was 14% to obtain a pressure-sensitive adhesive composition (13). The blending amount of the crosslinking agent is the number of parts by mass as an active component.

### (Example 50) Preparation of Pressure-Sensitive Adhesive Composition (14)

<Preparation of Acrylic Resin (A-3)>

In a reaction container equipped with a cooling tube, a nitrogen introduction tube, a thermometer, and a stirrer, a mixed solution of 81.8 parts of ethyl acetate as a solvent, 60 parts of methyl acrylate, 10 parts of 2-hydroxyethyl acrylate, 10 parts of acrylic acid, and 20 parts of 2-phenoxyethyl acrylate as monomers were charged, and the internal temperature was raised to 55° C. while air in the reaction container was replaced with nitrogen gas to exclude oxygen. Thereafter, a solution prepared by dissolving 0.14 parts of azobisisobutyronitrile (polymerization initiator) in 10 parts of ethyl acetate was added in its entirety. After the initiator was added, the mixture was kept at this temperature for 1 hour, and then ethyl acetate was continuously added into the reaction container at an addition rate of 17.3 parts/hr while the internal temperature was kept at 54 to 56° C., and when the concentration of the acrylic resin reached 35%, the addition of ethyl acetate was stopped, and the mixture was kept at this temperature until 12 hours had elapsed from the start of addition of ethyl acetate. Finally, ethyl acetate was added to adjust the concentration of the acrylic resin to 20%, thereby preparing an ethyl acetate solution of the acrylic resin. The obtained acrylic resin had a polystyrene equivalent weight average molecular weight Mw of 92 million as measured by GPC. The Mw/Mn was 7.8. This is referred to as an acrylic resin (A-3).

<Preparation of Pressure-Sensitive Adhesive Composition (14)>

A pressure-sensitive adhesive composition (14) was obtained in the same manner as that in Example 49 except that the acrylic resin (A-3) synthesized above was used instead of the acrylic resin (A-2).

### (Example 51) Preparation of Pressure-Sensitive Adhesive Composition (15)

<Preparation of Acrylic Resin (A-4)>

In a reaction container equipped with a cooling tube, a nitrogen introduction tube, a thermometer, and a stirrer, a mixed solution of 81.8 parts of ethyl acetate as a solvent, 10 parts of butyl acrylate, 60 parts of methyl acrylate, 10 parts of 2-hydroxyethyl acrylate, 10 parts of acrylic acid, and 10 parts of 2-phenoxyethyl acrylate as monomers were charged, and the internal temperature was raised to 55° C. while air in the reaction container was replaced with nitrogen gas to exclude oxygen. Thereafter, a solution prepared by dissolving 0.14 parts of azobisisobutyronitrile (polymerization initiator) in 10 parts of ethyl acetate was added in its entirety. After the initiator was added, the mixture was kept at this temperature for 1 hour, and then ethyl acetate was continuously added into the reaction container at an addition rate of 17.3 parts/hr while the internal temperature was kept at 54 to 56° C., and when the concentration of the acrylic resin reached 35%, the addition of ethyl acetate was stopped, and the mixture was kept at this temperature until 12 hours had elapsed from the start of addition of ethyl acetate. Finally, ethyl acetate was added to adjust the concentration of the acrylic resin to 20%, thereby preparing an ethyl acetate solution of the acrylic resin. The obtained acrylic resin had a polystyrene equivalent weight average molecular weight Mw of 94 million as measured by GPC. The Mw/Mn was=8.5. This is referred to as an acrylic resin (A-4).

<Preparation of Pressure-Sensitive Adhesive Composition (15)>

A pressure-sensitive adhesive composition (15) was obtained in the same manner as that in Example 49 except that the acrylic resin (A-4) synthesized above was used instead of the acrylic resin (A-2).

### (Example 52) Preparation of Pressure-Sensitive Adhesive Composition (16)

<Preparation of Acrylic Resin (A-5)>

In a reaction container equipped with a cooling tube, a nitrogen introduction tube, a thermometer, and a stirrer, a mixed solution of 81.8 parts of ethyl acetate as a solvent, 20 parts of butyl acrylate, 50 parts of methyl acrylate, 10 parts of 2-hydroxyethyl acrylate, 10 parts of acrylic acid, and 10 parts of 2-phenoxyethyl acrylate as monomers were charged, and the internal temperature was raised to 55° C. while air in the reaction container was replaced with nitrogen gas to exclude oxygen. Thereafter, a solution prepared by dissolving 0.14 parts of azobisisobutyronitrile (polymerization initiator) in 10 parts of ethyl acetate was added in its entirety. After the initiator was added, the mixture was kept at this temperature for 1 hour, and then ethyl acetate was continuously added into the reaction container at an addition rate of 17.3 parts/hr while the internal temperature was kept at 54 to 56° C., and when the concentration of the acrylic resin reached 35%, the addition of ethyl acetate was stopped, and the mixture was kept at this temperature until 12 hours had elapsed from the start of addition of ethyl acetate. Finally, ethyl acetate was added to adjust the concentration of the acrylic resin to 20%, thereby preparing an ethyl acetate solution of the acrylic resin. The obtained acrylic resin had a p styrene equivalent weight average molecular weight Mw of 91 million as measured by GPC. This is referred to as an acrylic resin (A-5).

<Preparation of Pressure-Sensitive Adhesive Composition (16)>

A pressure-sensitive adhesive composition (16) was obtained in the same manner as that in Example 49 except that the acrylic resin (A-5) synthesized above was used instead of the acrylic resin (A-2).

### (Example 53) Preparation of Pressure-Sensitive Adhesive Composition (17)

<Preparation of Acrylic Resin (A-6)>

In a reaction container equipped with a cooling tube, a nitrogen introduction tube, a thermometer, and a stirrer, a mixed solution of 81.8 parts of ethyl acetate as a solvent, 50 parts of butyl acrylate, 10 parts of methyl acrylate, 10 parts of 2-hydroxyethyl acrylate, 10 parts of acrylic acid, and 20 parts of 2-phenoxyethyl acrylate as monomers were charged, and the internal temperature was raised to 55° C. while air in the reaction container was replaced with nitrogen gas to exclude oxygen. Thereafter, a solution prepared by dissolving 0.14 parts of azobisisobutyronitrile (polymerization initiator) in 10 parts of ethyl acetate was added in its entirety. After the initiator was added, the mixture was kept at this temperature for 1 hour, and then ethyl acetate was continuously added into the reaction container at an addition rate of 17.3 parts/hr while the internal temperature was kept at 54 to 56° C., and when the concentration of the acrylic resin reached 35%, the addition of ethyl acetate was stopped, and the mixture was kept at this temperature until 12 hours had elapsed from the start of addition of ethyl acetate. Finally, ethyl acetate was added to adjust the concentration of the acrylic resin to 20%, thereby preparing an ethyl acetate solution of the acrylic resin. The obtained acrylic resin had a polystyrene equivalent weight average molecular weight Mw of 120 million as measured by GPC. This is referred to as an acrylic resin (A-6).

### Preparation of Pressure-Sensitive Adhesive Composition (17)

A pressure-sensitive adhesive composition (17) was obtained in the same manner as that in Example 49 except that the acrylic resin (A-6) synthesized above was used instead of the acrylic resin (A-2).

### (Example 54) Preparation of Pressure-Sensitive Adhesive Composition (18)

<Preparation of Acrylic Resin (A-7)>

In a reaction container equipped with a cooling tube, a nitrogen introduction tube, a thermometer, and a stirrer, a mixed solution of 81.8 parts of ethyl acetate as a solvent, 60 parts of butyl acrylate, 10 parts of methyl acrylate, 10 parts of 2-hydroxyethyl acrylate, 10 parts of acrylic acid, and 10 parts of 2-phenoxyethyl acrylate as monomers were charged, and the internal temperature was raised to 55° C. while air in the reaction container was replaced with nitrogen gas to exclude oxygen. Thereafter, a solution prepared by dissolving 0.14 parts of azobisisobutyronitrile (polymerization initiator) in 10 parts of ethyl acetate was added in its entirety. After the initiator was added, the mixture was kept at this temperature for 1 hour, and then ethyl acetate was continuously added into the reaction container at an addition rate of 17.3 parts/hr while the internal temperature was kept at 54 to 56° C., and when the concentration of the acrylic resin reached 35%, the addition of ethyl acetate was stopped, and the mixture was kept at this temperature until 12 hours had elapsed from the start of addition of ethyl acetate. Finally, ethyl acetate was added to adjust the concentration of the acrylic resin to 20%, thereby preparing an ethyl acetate solution of the acrylic resin. The obtained acrylic resin had a polystyrene equivalent weight average molecular weight Mw of 118 million as measured by GPC. This is referred to as an acrylic resin (A-7).

### Preparation of Pressure-Sensitive Adhesive Composition (18)

A pressure-sensitive adhesive composition (18) was obtained in the same manner as that in Example 49 except that the acrylic resin (A-7) synthesized above was used instead of the acrylic resin (A-2).

### (Example 55) Preparation of Pressure-Sensitive Adhesive Composition (19)

<Preparation of Acrylic Resin (A-8)>

In a reaction container equipped with a cooling tube, a nitrogen introduction tube, a thermometer, and a stirrer, a mixed solution of 81.8 parts of ethyl acetate as a solvent, 70 parts of butyl acrylate, 10 parts of 2-hydroxyethyl acrylate, 10 parts of acrylic acid, and 10 parts of 2-phenoxyethyl acrylate as monomers were charged, and the internal temperature was raised to 55° C. while air in the reaction container was replaced with nitrogen gas to exclude oxygen. Thereafter, a solution prepared by dissolving 0.14 parts of azobisisobutyronitrile (polymerization initiator) in 10 parts of ethyl acetate was added in its entirety. After the initiator was added, the mixture was kept at this temperature for 1 hour, and then ethyl acetate was continuously added into the reaction container at an addition rate of 17.3 parts/hr while the internal temperature was kept at 54 to 56° C., and when the concentration of the acrylic resin reached 35%, the addition of ethyl acetate was stopped, and the mixture was kept at this temperature until 12 hours had elapsed from the start of addition of ethyl acetate. Finally, ethyl acetate was added to adjust the concentration of the acrylic resin to 20%, thereby preparing an ethyl acetate solution of the acrylic resin. The obtained acrylic resin had a polystyrene equivalent weight average molecular weight Mw of 110 million as measured by GPC. This is referred to as an acrylic resin (A-8).

<Preparation of Pressure-Sensitive Adhesive Composition (19)>

A pressure-sensitive adhesive composition (19) was obtained in the same manner as that in Example 49 except that the acrylic resin (A-8) synthesized above was used instead of the acrylic resin (A-2).

<Evaluation of Crystal Precipitation (Bleeding Resistance) of Pressure-Sensitive Adhesive Layer>

The pressure-sensitive adhesive composition (13) was applied to a release-treated surface of a separator film [trade name “PLR-382190” available from Lintec Corporation] made of a release-treated polyethylene terephthalate film using an applicator, and dried at 100° C. for 1 minute to prepare a pressure-sensitive adhesive layer. A separate film was further laminated on the other surface of the pressure-sensitive adhesive layer to obtain a pressure-sensitive adhesive layer with a separate film on both sides. The thickness of the obtained pressure-sensitive adhesive layer was 15 μm.

The obtained pressure-sensitive adhesive layer with a separate film on both sides was cured for 7 days under the conditions of a temperature of 23° C. and a relative humidity of 65%. Using the pressure-sensitive adhesive layer with a separate film on both sides after curing, the presence or absence of in-plane crystal precipitation of the compound was confirmed using a microscope. A case where there was no crystal precipitation was evaluated as a, and a case where there was crystal precipitation was evaluated as b. The evaluation results are shown in the column of “after curing” in Table 7.

The obtained pressure-sensitive adhesive layer with a separate film on both sides was stored under air at a temperature of 40° C. for 1 month. Using the pressure-sensitive adhesive layer with a separate film on both sides after storage, the presence or absence of in-plane crystal precipitation of the compound was confirmed using a microscope. A case where there was no crystal precipitation was evaluated as a, and a case where there was crystal precipitation was evaluated as b. The evaluation results are shown in the column of “40° C. 1 N” in Table 7.

The presence or absence of crystal precipitation was confirmed in the same manner except that the pressure-sensitive adhesive composition (13) was changed to the pressure-sensitive adhesive composition (14) to the pressure-sensitive adhesive composition (19). The results are shown in Table 7.

| | Pressure-sensitive | | | |
|---|---|---|---|---|
| | adhesive | | Crystal precipitation | |
| | composition | Acrylic resin | After curing | 40° C. 1M |
| Example 49 | (13) | (A-2) | b | — |
| Example 50 | (14) | (A-3) | a | a |
| Example 51 | (15) | (A-4) | a | a |
| Example 52 | (16) | (A-5) | a | a |
| Example 53 | (17) | (A-6) | a | b |
| Example 54 | (18) | (A-7) | a | b |
| Example 55 | (15) | (A-9) | a | b |

### (Example 56) Preparation of Pressure-Sensitive Adhesive Layer (12) and Pressure-Sensitive Adhesive Sheet (12)

The obtained pressure-sensitive adhesive composition (13) was applied to a release-treated surface of a separator film [trade name “PLR-382190” available from Lintec Corporation] made of a release-treated polyethylene terephthalate film using an applicator, and dried at 100° C. for 1 minute to prepare a pressure-sensitive adhesive layer (12). The thickness of the obtained pressure-sensitive adhesive layer was 15 μm.

The obtained pressure-sensitive adhesive layer (12) was bonded to a cycloolefin film not containing an ultraviolet absorber of 23 μm by a laminator, and then aged for 7 days under conditions of a temperature of 23° C. and a relative humidity of 65% to obtain a pressure-sensitive adhesive sheet (12).

### (Example 57) Preparation of Pressure-Sensitive Adhesive Layer (13) and Pressure-Sensitive Adhesive Sheet (13)

A pressure-sensitive adhesive layer (13) and a pressure-sensitive adhesive sheet (13) were produced in the same manner as in Example 56 except that the pressure-sensitive adhesive composition (13) was changed to the pressure-sensitive adhesive composition (14).

### (Example 58) Preparation of Pressure-Sensitive Adhesive Layer (14) and Pressure-Sensitive Adhesive Sheet (14)

A pressure-sensitive adhesive layer (14) and a pressure-sensitive adhesive sheet (14) were prepared in the same manner as in Example 56 except that the pressure-sensitive adhesive composition (13) was changed to the pressure-sensitive adhesive composition (15).

### (Example 59) Preparation of Pressure-Sensitive Adhesive Layer (15) and Pressure-Sensitive Adhesive Sheet (15)

A pressure-sensitive adhesive layer (15) and a pressure-sensitive adhesive sheet (15) were prepared in the same manner as in Example 56 except that the pressure-sensitive adhesive composition (13) was changed to the pressure-sensitive adhesive composition (16).

### (Example 60) Preparation of Pressure-Sensitive Adhesive Layer (16) and Pressure-Sensitive Adhesive Sheet (16)

A pressure-sensitive adhesive layer (16) and a pressure-sensitive adhesive sheet (16) were produced in the same manner as in Example 56 except that the pressure-sensitive adhesive composition (13) was changed to the pressure-sensitive adhesive composition (17).

### (Example 61) Preparation of Pressure-Sensitive Adhesive Layer (17) and Pressure-Sensitive Adhesive Sheet (17)

A pressure-sensitive adhesive layer (17) and a pressure-sensitive adhesive sheet (17) were prepared in the same manner as in Example 56 except that the pressure-sensitive adhesive composition (13) was changed to the pressure-sensitive adhesive composition (18).

### (Example 62) Preparation of Pressure-Sensitive Adhesive Layer (18) and Pressure-Sensitive Adhesive Sheet (18)

A pressure-sensitive adhesive layer (18) and a pressure-sensitive adhesive sheet (18) were prepared in the same manner as in Example 56 except that the pressure-sensitive adhesive composition (13) was changed to the pressure-sensitive adhesive composition (19).

<Measurement of Absorbance Retention of Pressure-Sensitive Adhesive Sheet>

The obtained pressure-sensitive adhesive sheet (12) was cut into a size of 30 mm×30 mm, the separate film was peeled off, and the pressure-sensitive adhesive layer (12) and alkali-free glass [trade name “EAGLE XG” produced by Corning Incorporated] were bonded to each other to prepare a sample (5). The absorbance of the prepared sample (5) in the wavelength range of 300 to 800 nm was measured every 1 nm step using a spectrophotometer (UV-2450: manufactured by Shimadzu Corporation). The measured absorbances at a wavelength of 400 nm were taken as the absorbances at a wavelength of 400 nm of the pressure-sensitive adhesive sheet (12). The results are shown in Table 8. Both the cycloolefin film alone and the non-alkali glass alone have zero absorbance at a wavelength of 400 nm.

The sample (5) after the absorbance measurement was charged into a Sunshine weathermeter (manufactured by Suga Test Instruments Co., Ltd.) for 150 hours under the conditions of a temperature of 63° C. and a relative humidity of 50% RH to perform a weather resistance test. The absorbance of the sample (5) taken out was measured in the same manner as described above. From the measured absorbance, the absorbance retention of the sample at a wavelength of 400 nm was determined based on the following formula. The results are shown in Table 8. A value of the absorbance retention closer to 100 indicates that the light selective absorption function is not deteriorated and good weather resistance is obtained.

The absorbance retention was also determined when the sample (5) was charged into a Sunshine weathermeter (manufactured by Suga Test instruments Co., Ltd.) for 225 hours under the conditions of a temperature of 63° C. and a relative humidity of 50% RH.

Absorbance retention (%)=(A(400) after durability test/A(400) before durability test)×100

The absorbance retention was measured in the same manner except that the pressure-sensitive adhesive sheet (12) was changed to the pressure-sensitive adhesive sheet (13) to the pressure-sensitive adhesive sheet (18). The results are shown in Table 8.

| | Pressure- | | | | |
|---|---|---|---|---|---|
| | sensitive | Pressure- | | | |
| | adhesive | sensitive | Acrylic | Absorbance | retention |
| | sheet | composition | resin, | 150 hr | 225 hr |
| Example 56 | (12) | (18) | (A-2) | 34.9 | 17.1 |
| Example 57 | (13) | (19) | (A-3) | 54.5 | 40.4 |
| Example 58 | (14) | (20) | (A-4) | 52.2 | 40.9 |
| Example 59 | (15) | (21) | (A-5) | 54 | 39.2 |
| Example 60 | (16) | (22) | (A-6) | 38.8 | 23.1 |
| Example 61 | (17) | (23) | (A-7) | 53.3 | 35 |
| Example 62 | (13) | (24) | (A-8) | 51.8 | 35.4 |

### (Example 63) Preparation of Pressure-Sensitive Adhesive Sheet (19)

A pressure-sensitive adhesive sheet (19) was prepared in the same manner as that in Example 56 except that the cycloolefin film not containing an ultraviolet absorber of 23 μm was changed to a cycloolefin film containing an ultraviolet absorber of 23 μm.

### (Example 64) Preparation of Pressure-Sensitive Adhesive Sheet (20)

A pressure-sensitive adhesive sheet (20) was prepared in the same manner as that in Example 57 except that the cycloolefin film not containing an ultraviolet absorber of 23 μm was changed to a cycloolefin film containing an ultraviolet absorber of 23 μm.

### (Example 65) Preparation of Pressure-Sensitive Adhesive Sheet (21)

A pressure-sensitive adhesive sheet (21) was prepared in the same manner as that in Example 58 except that the cycloolefin film not containing an ultraviolet absorber of 23 μm was changed to a cycloolefin film containing an ultraviolet absorber of 23 μm.

### (Example 66) Preparation of Pressure-Sensitive Adhesive Sheet (22)

A pressure-sensitive adhesive sheet (22) was prepared in the same manner as that in Example 59 except that the cycloolefin film not containing an ultraviolet absorber of 23 μm was changed to a cycloolefin film containing an ultraviolet absorber of 23 μm.

### (Example 67) Preparation of Pressure-Sensitive Adhesive Sheet (23)

A pressure-sensitive adhesive sheet (23) was prepared in the same manner as that in Example 60 except that the cycloolefin film not containing an ultraviolet absorber of 23 μm was changed to a cycloolefin film containing an ultraviolet absorber of 23 μm.

### (Example 68) Preparation of Pressure-Sensitive Adhesive Sheet (24)

A pressure-sensitive adhesive sheet (24) was prepared in the same manner as that in Example 61 except that the cycloolefin film not containing an ultraviolet absorber of 23 μm was changed to a cycloolefin film containing an ultraviolet absorber of 23 μm.

### (Example 69) Preparation of Pressure-Sensitive Adhesive Sheet (25)

A pressure-sensitive adhesive sheet (25) was prepared in the same manner as that in Example 62 except that the cycloolefin film not containing an ultraviolet absorber of 23 μm was changed to a cycloolefin film containing an ultraviolet absorber of 23 μm.

<Measurement of Absorbance Retention of Pressure-Sensitive Adhesive Sheet>

The obtained pressure-sensitive adhesive sheet (19) was cut into a size of 30 mm×30 mm, the separate film was peeled off, and the pressure-sensitive adhesive layer (19) and alkali-free glass [trade name “EAGLE XG” produced by Corning incorporated] were bonded to each other to prepare a sample (6). The absorbance of the prepared sample (5) in the wavelength range of 300 to 800 nm was measured every 1 nm step using a spectrophotometer (UV-2450: manufactured by Shimadzu Corporation). The measured absorbances at a wavelength of 405 nm were taken as the absorbances at a wavelength of 405 nm of the pressure-sensitive adhesive sheet (19). The results are shown in Table 9. The absorbance at a wavelength of 405 nm of the alkali-free glass alone is 0.

The sample (6) after the absorbance measurement was charged into a Sunshine weathermeter (manufactured by Suga Test Instruments Co., Ltd.) for 150 hours under the conditions of a temperature of 63° C. and a relative humidity of 50% RH to perform a weather resistance test. The absorbance of the sample (5) taken out was measured in the same manner as described above. From the measured absorbance, the absorbance retention of the sample at a wavelength of 405 nm was determined based on the following formula. The results are shown in Table 9. A value of the absorbance retention closer to 100 indicates that the light selective absorption function is not deteriorated and good weather resistance is obtained.

The absorbance retention was also determined when the sample (6) was charged into a Sunshine weathermeter (manufactured by Suga Test Instruments Co., Ltd.) for 225 hours under the conditions of a temperature of 63° C. and a relative humidity of 50% RH.

Absorbance retention (5)=(A(405) after durability test/A(405) before durability test)×100

The absorbance retention was measured in the same manner except that the pressure-sensitive adhesive sheet (19) was changed to the pressure-sensitive adhesive sheet (20) to the pressure-sensitive adhesive sheet (25). The results are shown in Table 9.

| | Pressure- | Pressure- | | | |
|---|---|---|---|---|---|
| | sensitive | sensitive | | | |
| | adhesive | adhesive | Acrylic | Absorbance retention | |
| | sheet | composition | resin, | 150 hr | 225 hr |
| Example 63 | (19) | (18) | (A-2) | 19.2 | 33.5 |
| Example 64 | (20) | (19) | (A-3) | 33.3 | 80.2 |
| Example 65 | (21) | (20) | (A-4) | 71.1 | 68.3 |
| Example 66 | (22) | (21) | (A-5) | 71 | 63.4 |
| Example 67 | (23) | (22) | (A-6) | 66.6 | 62.3 |
| Example 68 | (24) | (23) | (A-7) | 34.5 | 82.8 |
| Example 69 | (25) | (24) | (A-9) | 33.9 | 81.7 |

### Example 70

<Preparation of Resin Composition for Spectacle Lens>

40 parts of xylylene diisocyanate, 60 parts of trimethylolpropane tris(thioglycolate), 1.6 parts of a compound represented by Formula (UVA-6), 0.2 parts of a release agent (trade name: ZELEC-UN, available from Sigme-Aldrich), and 0.03 parts of dibutyldichlorotin were mixed and stirred. The obtained mixture was allowed to stand in a vacuum dryer for 1 hour and degassed. The obtained mixture was poured into a glass mold and heated at 120° C. for 1 hour. Only a resin plate was peeled off to prepare a resin plate having a thickness of 2 mm and 3 cm×3 cm.

<Measurement of Absorbance Retention of Resin Plate>

The absorbance of the resin plate obtained above in a wavelength range of 300 to 800 nm was measured every 1 nm step by using a spectrophotometer (UV-2450: manufactured by Shimadzu Corporation).

The resin plate after the measurement was charged into a Sunshine weathermeter (manufactured by Suga Test Instruments Co., Ltd.) for 75 hours under the conditions of a temperature of 63° C. and a relative humidity of 50% RH to perform a weather resistance test. The absorbance of the resin plate taken out was measured in the same manner as described above. From the measured absorbance, the absorbance retention of the sample at a wavelength of 420 nm was determined based on the following formula. The results are shown in Table 10. A value of the absorbance retention closer to 100 indicates that the light selective absorption function is not deteriorated and good weather resistance is obtained.

Note that the spectacle lens is required to have a good absorbance retention at a wavelength of 420 nm in order to efficiently cut light of blue light that tends to have a negative effect on health. As the value of A (420)/A (480) is larger, the blue light can be cut with less coloring.

Absorbance retention (%)=(A(420) after durability test/A(420) before durability test)×100

| | | | | A(420)/ | Absorbance |
|---|---|---|---|---|---|
| | Compounds | A(420) | A(480) | A(480) | retention |
| Example 70 | Formula | 3.26 | 0.049 | 66.5 | 100 |
| | (UVA-6) | | | | |

The compound of the present invention has high absorption selectivity to short-wavelength visible light having a wavelength of 380 to 400 nm. In addition, the compound of the present invention has a high absorbance retention even after a weather resistance test, and has good weather resistance.

1. A compound represented by Formula (I): 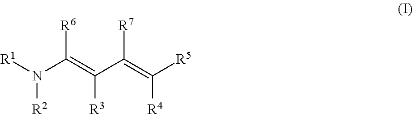
[in Formula (I),
R³, R⁴, and R⁵ each independently represent an electron-withdrawing group;
R¹, R², R⁶ and R⁷ each independently represent a hydrogen atom, a heterocyclic group, a halogen atom, a nitro group, a cyano group, a hydroxy group, a thiol group, a carboxy group, —SCF₃, —SF₅, —SF₃, —SO₃H, —SO₂H, an aliphatic hydrocarbon group having 1 to 25 carbon atoms which may have a substituent, or an aromatic hydrocarbon group having 6 to 18 carbon atoms which may have a substituent, and —CH₂— or —CH═ contained in the aliphatic hydrocarbon group or the aromatic hydrocarbon group may be substituted with —NR^{1A}—, —SO₂—, —CO—, —O—, —COO—, —OCO—, —CONR^{2A}—, —NR^{3A}—CO—, —S—, —SO—, —CF₂— or —CHF—;
R^{1A}, R^{2A}, and R^{3A} each independently represent a hydrogen atom or an alkyl group having 1 to 6 carbon atoms;
R¹ and R² may be linked to each other to form a ring;
R¹ and R⁶ may be linked to each other to form a ring;
R⁶ and R⁷ may be linked to each other to form a ring; and
R⁴ and R⁵ may be linked to each other to form a ring].

2. The compound according to claim 1, wherein R³ is a nitro group, a cyano group, —F, —OCF₃, —SCF₃, —SF₅, —SF₃, —SO₂—R²²² (R²²² represents a hydrogen atom, an alkyl group having 1 to 25 carbon atoms which may have a substituent, or an aromatic hydrocarbon group having 6 to 18 carbon atoms which may have a substituent), a fluoroalkyl group, or a fluoroaryl group.

3. The compound according to claim 1, wherein R³ is a cyano group.

4. The compound according to claim 1, wherein at least one selected from R⁴ and R⁵ is a cyano group, a nitro group, —OCF₃, —SCF₃, —SF₅, —CO—O—R²²², —SO₂—R²²² (R²²² represents a hydrogen atom, an alkyl group having 1 to 25 carbon atoms which may have a substituent, or an aromatic hydrocarbon group having 6 to 18 carbon atoms which may have a substituent), a fluoroalkyl group, or a fluoroaryl group.

5. The compound according to claim 1, wherein at least one selected from R⁴ and R⁵ is a cyano group.

6. The compound according to claim 1, wherein both R⁴ and R⁵ are a cyano group.

7. The compound according to claim 1, wherein R¹ and R² are each independently an aliphatic hydrocarbon group which may have a substituent.

8. The compound according to claim 1, wherein R¹ and R² are linked to each other to form a ring.

9. The compound according to claim 8, wherein the ring formed by linking R¹ and R² to each other is a ring having no unsaturated bond.

10. The compound according to claim 1, wherein the compound represented by Formula (I) is a compound represented by Formula (II): 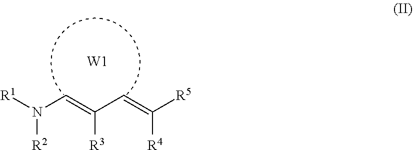
[in the formula, R¹, R², R³, R⁴, and R⁵ have the same meaning as described above; and
a ring W¹ represents a ring structure having at least one double bond as a constituent element of the ring and having no aromaticity].

11. The compound according to claim 10, wherein the ring W¹ has a 5 to 7-membered ring structure.

12. The compound according to claim 10, wherein the ring W¹ is a 6-membered ring structure.

13. The compound of claim 1, wherein λ max≥370 nm,
(λ max represents a maximum absorption wavelength [nm] of the compound represented by Formula (I)).

14. The compound according to claim 1, which satisfies Formula (B),
ε(λ max)/ε(λ max+30 nm)≥5 (B)
(in the formula, ε (λ max) represents a gram absorption coefficient at a maximum absorption wavelength [nm] of the compound represented by Formula (I), and ε (λ max+30 nm) represents a gram absorption coefficient at a wavelength [nm] of (maximum absorption wavelength [nm]+30 nm) of the compound represented by Formula (I)).

15. A composition containing the compound according to claim 1.
